(19) United States
(12) Patent Application Publication
(10) Pub. No.: US 2025/0195835 A1
(43) Pub. Date: Jun. 19, 2025

(54) ROBOTIC HUB ASSEMBLY

(71) Applicant: Imperative Care, Inc., Campbell, CA (US)

(72) Inventors: Sean Totten, Kirkland, WA (US); Craig Mar, Fremont, CA (US); Jason Lee, Seattle, WA (US); Kyle Bartholomew, Campbell, CA (US); Cedric Bathany, Los Gatos, CA (US)

(21) Appl. No.: 18/986,519

(22) Filed: Dec. 18, 2024

Related U.S. Application Data
(60) Provisional application No. 63/612,272, filed on Dec. 19, 2023.

(51) Int. Cl. A61M 25/00; A61B 34/30; A61M 39/06; A61M 39/10

(52) U.S. Cl. CPC A61M 25/0097; A61B 34/30; A61M 39/06; A61M 39/10; A61B 2034/301

(57) ABSTRACT

A hub assembly includes a mount being axially movable along a drive surface, a catheter hub having a catheter, the catheter hub removably couplable with the mount, and a fluidics connector in fluid communication with the mount and the catheter hub to provide fluid and vacuum through the connector to the catheter hub.

### INCORPORATION BY REFERENCE TO ANY PRIORITY APPLICATIONS

Any and all applications for which a foreign or domestic priority claim is identified in the Application Data Sheet filed with the present application are hereby incorporated by reference under 37 C.F.R. § 1.57. The present application claims priority to U.S. Provisional Patent Application No. 63/612,272, filed Dec. 19, 2023, titled ROBOTIC CATHETER HUB ASSEMBLY, the entire content of which is incorporated by reference herein for all purposes and forms a part of this specification.

### BACKGROUND

### Field

The present application relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neuro interventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovasculature is challenging to achieve, especially Type III arches. consuming and requires guidewire and access catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

There is provided in accordance with one aspect of the present disclosure a supra-aortic access robotic control system. The system includes a guidewire hub configured to adjust each of an axial position and a rotational position of a guidewire; a guide catheter hub configured to adjust a guide catheter in an axial direction; and an access catheter hub configured to adjust each of an axial position and a rotational position of an access catheter. The access catheter hub may also laterally deflect a distal deflection zone of the access catheter. The guidewire hub may additionally be configured to laterally deflect a distal portion of the guidewire.

There may also be provided a procedure catheter hub configured to manipulate a procedure catheter. Following robotic placement of the guidewire, access catheter and guide catheter such that the guide catheter achieves supra aortic access, the guidewire and access catheter may be proximally withdrawn and the procedure catheter advanced through and beyond the guide catheter, with or without guidewire support (said guidewire may be smaller in diameter and/or more flexible than the guidewire used to gain supra aortic access), to reach a more distal neurovascular treatment site. The procedure catheter may be an aspiration catheter; an embolic deployment catheter; a stent deployment catheter; a flow diverter deployment catheter, an access catheter; a diagnostic angiographic catheter; a guiding catheter, an imaging catheter, a physiological sensing/measuring catheter, an infusion or injection catheter, an ablation catheter, an RF ablation catheter or guidewire, a balloon catheter, or a microcatheter used to deliver a stent retriever, a balloon catheter or a stent retriever.

The control system may further include a driven magnet on each of a guidewire hub, an access catheter hub and a guide catheter hub, configured to cooperate with corresponding drive magnets such that the driven magnet moves in response to movement of the corresponding drive magnet. The drive magnets may each be independently axially movably carried by a support table. The drive magnets may be located outside of the sterile field, separated from the driven magnets by a barrier, and the driven magnets may be within the sterile field. The barrier may include a tray made from a thin polymer membrane, or any membrane of non-ferromagnetic material.

The control system may further include a control console which may be connected to the support table or may be located remotely from the support table. The position of each driven magnet and corresponding hub is movable in response to manual manipulation of a guidewire drive control, access catheter drive control, or procedure catheter drive control on the console or on a particular controller not associated with the console.

The control system may further include a processor for controlling the position of the drive magnets. The processor may be in wired communication with the control console, or in wireless communication with the control console. The driven magnets may be configured to remain engaged with the corresponding drive magnets until application of an axial disruption force of at least about 300 grams.

There is also provided a robotically driven interventional device. The device includes an elongate, flexible body, having a proximal end and a distal end. A hub is provided on the proximal end. At least one rotatable roller is provided on a first surface of the hub; and at least one magnet is provided on the first surface of the hub. The roller may extend further away from the first surface than the magnet. The hub may be further provided with at least a second roller.

Any of the guidewire hub, access catheter hub and procedure catheter hub may be further provided with a rotational drive, for rotating the corresponding interventional device with respect to the hub. The hub may be further provided with an axial drive mechanism to distally advance or proximally retract a control element extending axially through the interventional device, to adjust a characteristic such as shape or flexibility of the interventional device. In some embodiments, at least one control element may be an axially movable tubular body or fiber, ribbon, or wire such as a pull wire extending through the interventional device to, for example, a distal deflection zone. In some embodiments, any number of control elements may be advanced, retracted, or otherwise moved in a similar manner.

There is also provided a control system for controlling movement of interventional devices. In one configuration, the control system includes a guidewire control, configured to control axial travel and rotation of a guidewire; an access catheter control, configured to control axial and rotational movement of an access catheter; and a guide catheter control, configured to control axial movement and/or rotation of a guide catheter.

The control system may further include a deflection control, configured to control deflection of the access catheter or procedure catheter, and may be configured for wired or wireless communication with a robotic catheter drive system.

The control system may be configured to independently control the three or more hubs in a variety of modes. For example, two or more hubs may be selectively ganged together so that they drive the respective devices simultaneously and with the same motion. Alternatively, the control system may be configured to drive respective devices simultaneously but with different motions.

The control system may further include a physician interface for operating the control system. The physician interface may be carried by a support table having a robotic interventional device drive system. Alternatively, the physician interface for operating the control system may be carried on a portable, handheld device or desktop computer, and may be located in the same room as the patient, the same facility as the patient, or in a remote facility.

The control system may further include a graphical user interface with at least one display for indicating the status of at least one device parameter, and/or indicating the status of at least one patient parameter.

There is also provided a sterile packaging assembly for transporting interventional devices to a robotic surgery site. The packaging assembly may include a base and a sterile barrier configured to enclose a sterile volume. At least one interventional device may be provided within the sterile volume, the device including a hub and an elongate flexible body. The hub may include at least one magnet and at least one roller configured to roll on the base.

In one implementation, the sterile barrier is removably attached to the base to define the enclosed volume between the sterile barrier and the base. In another implementation, the sterile barrier is in the form of a tubular enclosure for enclosing the sterile volume. The tubular enclosure may surround the base and the at least one interventional device, which are within the sterile volume.

The hub may be oriented within the packaging such that the roller and the magnet face the base. Alternatively, the base may be in the form of a tray having an elongate central axis. An upper, sterile field side of the tray may have an elongate support surface for supporting and permitting sliding movement of one or more hubs. At least one and optionally two elongate trays may be provided, extending parallel to the central axis. At least one hub and interventional device may be provided in the tray, and the sterile tray with sterile hub and interventional device may be positioned in a sterile volume defined by a sterile barrier.

The base may be configured to reside on a support table adjacent a patient, with an upper surface of the base within a sterile field and a lower surface of the base outside of the sterile field.

Any of the hubs disclosed herein may further include a fluid injection port and/or a wireless RF transceiver for communications and/or power transfer. The hub may include a visual indicator, for indicating the presence of a clot. In some embodiments, the hub may also include wired electrical communications and power port. The visual indicator may include a clot chamber having a transparent window. A filter may be provided in the clot chamber.

Any of the hubs disclosed herein may further include a sensor for detecting a parameter of interest such as the presence of a clot. The sensor, in some instances, may be positioned on a flexible body. The sensor may include a pressure sensor or an optical sensor. In some embodiments, the sensor may include one or more of a force sensor, a positioning sensor, a temperature sensor, and/or an oxygen sensor. In some embodiments, the sensor may include a Fiber Bragg grating sensor. For example, a Fiber Bragg grating sensor (e.g., an optical fiber) may detect strain locally that can facilitate the detection and/or determination of force being applied. The device may further include a plurality of sensors. The plurality of sensors may each include one or more of any type of sensor disclosed herein. In some embodiments, a plurality (e.g., 3 or more) of sensors (e.g., Fiber Bragg grating sensors) may be distributed around a perimeter to facilitate the detection and/or determination of shape. The position of the device, in some instance, may be determined through the use of one or more sensors to detect and/or determine the position. For example, one or more optical encoders may be located in or proximate to one or more the motors that drive linear motion such that the optical encoders may determine a position.

There is also provided a method of performing a neurovascular procedure, in which a first phase includes robotically achieving supra-aortic access, and a second phase includes manually or robotically performing a neurovascular procedure via the supra-aortic access. The method includes the steps of providing an access catheter having an access catheter hub; coupling the access catheter hub to a hub adapter movably carried by a support table; driving the access catheter in response to movement of the hub adapter along the table until the access catheter is positioned to achieve supra-aortic access. The access catheter and access catheter hub may then be decoupled from the hub adapter; and a procedure catheter hub having a procedure catheter may then be coupled to the hub adapter.

The method may additionally include advancing the procedure catheter hub to position a distal end of the procedure catheter at a neurovascular treatment site. The driving the access catheter step may include driving the access catheter distally through a guide catheter. The driving the access catheter step may include the step of laterally deflecting a distal region of the access catheter to achieve supra-aortic access. In some embodiments, the driving the access catheter step may also include rotating the access catheter.

There is also provided a method of performing a neurovascular procedure, comprising the steps of providing an access assembly comprising a guidewire, access catheter and guide catheter. The access assembly may be releasably coupled to a robotic drive system. The access assembly may be driven by the robotic drive system to achieve access to a desired point, such as to achieve supra-aortic access. The guidewire and the access catheter may then be decoupled from the access assembly, leaving the guide catheter in place. A procedure assembly may be provided, comprising at least a guidewire and a first procedure catheter. The procedure assembly may be releasably coupled to the robotic drive system; and a neurovascular procedure may be accomplished using the procedure assembly. A second procedure catheter may also be provided, for extending through the first procedure catheter to a treatment site.

The coupling the access assembly step may include magnetically coupling a hub on each of the guidewire, access catheter and guide catheter, to separate corresponding couplers carrying corresponding drive magnets independently movably carried by the drive table. The procedure assembly may include a guidewire, a first catheter and a second catheter. The guidewire and first catheter may be positioned concentrically within the second catheter. The procedure assembly may be advanced as a unit through at least a portion of the length of the guide catheter, and the procedure may include a neurovascular thrombectomy.

There is also provided a method of performing a neurovascular procedure. The method includes the steps of providing a multi-catheter assembly including an access catheter, a guide catheter, and a procedure catheter, coupling the assembly to a robotic drive system, driving the assembly to achieve supra-aortic access, driving a subset of the assembly to a neurovascular site, wherein the subset includes the guide catheter and the procedure catheter, proximally removing the access catheter, and performing a neurovascular procedure using the procedure catheter.

The neurovascular procedure can include a neurovascular thrombectomy. The assembly may further include a guidewire, wherein each of the guidewire, the access catheter, the guide catheter, and the procedure catheter are configured to be adjusted by a respective hub. Coupling the assembly to the robotic drive system can include magnetically coupling a first hub of the guidewire to a first drive magnet, magnetically coupling a second hub of the access catheter to a second drive magnet, magnetically coupling a third hub of the guide catheter to a third drive magnet, and magnetically coupling a fourth hub of the procedure catheter to a fourth drive magnet. The first drive magnet, the second drive magnet, the third drive magnet, and the fourth drive magnet can each be independently movably carried by a drive table. The procedure catheter can be an aspiration catheter. The procedure catheter can be an embolic deployment catheter. The procedure catheter can be a stent deployment catheter. The procedure catheter can be a flow diverter deployment catheter. The procedure catheter can be a diagnostic angiographic catheter. The procedure catheter can be a stent retriever catheter. The procedure catheter can be a clot retriever. The procedure catheter can be a balloon catheter. The procedure catheter can be a catheter to facilitate percutaneous valve repair or replacement. The procedure catheter can be an ablation catheter.

There is also provided a method of performing a neurovascular procedure. The method includes the steps of providing an assembly including a guidewire, an access catheter, a guide catheter, and a procedure catheter coaxially moveably assembled into a single multicatheter assembly, coupling the assembly to a drive system, driving the assembly to achieve supra-aortic access, driving a subset of the assembly to an intracranial site, wherein the subset includes the guidewire, the guide catheter, and the procedure catheter, and performing a neurovascular procedure using the subset of the assembly.

Each of the guidewire, the access catheter, the guide catheter, and the procedure catheter can be configured to be adjusted by a respective hub. Coupling the assembly to the drive system can include magnetically coupling a first hub of the guidewire to a first drive magnet, magnetically coupling a second hub of the access catheter to a second drive magnet, magnetically coupling a third hub of the guide catheter to a third drive magnet, and magnetically coupling a fourth hub of the procedure catheter to a fourth drive magnet. The drive system can be a robotic drive system, and the first drive magnet, the second drive magnet, the third drive magnet, and the fourth drive magnet can each be independently movably carried by a drive table associated with the robotic drive system. The first drive magnet, the second drive magnet, the third drive magnet, and the fourth drive magnet can each be independently movably carried by a drive table.

There is also provided a method of performing a neurovascular procedure. The method includes providing an assembly including a guidewire having a guidewire hub, an access catheter having an access catheter hub, and a guide catheter having a guide catheter hub. The method also includes coupling the guidewire hub to a first hub adapter, the access catheter hub to a second hub adapter, and the guide catheter hub to a third hub adapter, wherein each of the first hub adapter, the second hub adapter and the third hub adapter is movably carried by a support table. The method also includes driving the assembly in response to movement of each of the first hub adapter, the second hub adapter and the third hub adapter along the support table until the assembly is positioned to achieve supra-aortic vessel access.

The method can include the step of driving a subset of the assembly along the support table until the subset of the assembly is positioned to perform a neurovascular procedure at a neurovascular treatment site, wherein the subset of the assembly includes the guidewire, the guide catheter, and a procedure catheter. The neurovascular procedure can include a thrombectomy. Coupling the guidewire hub to the first hub adapter can include magnetically coupling the guidewire hub to a first drive magnet. Coupling the access catheter hub to the second hub adapter can include magnetically coupling the access catheter hub to a second drive magnet. Coupling the guide catheter hub to the third hub adapter can include magnetically coupling the guide catheter hub to a third drive magnet. The first drive magnet, the second drive magnet and the third drive magnets can be independently movably carried by the support table. The first drive magnet can be coupled to a first driven magnet across a sterile field barrier. The second drive magnet can be coupled to a second driven magnet across the sterile field barrier. The third drive magnet can be coupled to a third driven magnet across the sterile field barrier. Coupling the guidewire hub to the first hub adapter can include mechanically coupling the guidewire hub to a first drive. Coupling the access catheter hub to the second hub adapter can include mechanically coupling the access catheter hub to a second drive. Coupling the guide catheter hub to the third hub adapter can include mechanically coupling the guide catheter hub to a third drive. The guidewire and the guide catheter can be advanced as a unit along at least a portion of a length of the access catheter after supra-aortic access is achieved. The guidewire hub can be configured to adjust an axial position and a rotational position of the guidewire. The assembly can further include a procedure catheter having a procedure catheter hub. The procedure catheter hub can be configured to adjust an axial position and a rotational position of the procedure catheter. The procedure catheter hub can be further configured to laterally deflect a distal deflection zone of the procedure catheter. The guidewire hub can be configured to adjust an axial position and a rotational position of the guidewire. The procedure catheter hub can be configured to adjust an axial position and a rotational position of the procedure catheter. The guide catheter hub can be configured to adjust an axial position of the guide catheter. The access catheter hub can be configured to adjust an axial position and a rotational position of the access catheter. The procedure catheter hub can be further configured to laterally deflect a distal deflection zone of the procedure catheter. The access catheter hub can be further configured to laterally deflect a distal deflection zone of the access catheter. The guide catheter hub can be configured to adjust an axial position of the guide catheter. The access catheter hub can be configured to adjust an axial position and a rotational position of the access catheter. The access catheter hub can be further configured to laterally deflect a distal deflection zone of the access catheter.

There is also provided a drive system for achieving supra-aortic access and neurovascular treatment site access. The system includes a guidewire hub configured to adjust an axial position and a rotational position of a guidewire, a procedure catheter hub configured to adjust an axial position and a rotational position of a procedure catheter, a guide catheter hub configured to adjust an axial position of a guide catheter, and an access catheter hub configured to adjust an axial position and a rotational position of an access catheter, the access catheter further configured to laterally deflect a distal deflection zone of the access catheter.

The procedure catheter hub can be further configured to laterally deflect a distal deflection zone of the procedure catheter. The guidewire hub can be configured to couple to a guidewire hub adapter by magnetically coupling the guidewire hub to a first drive magnet. The access catheter hub can be configured to couple to an access catheter hub adapter by magnetically coupling the access catheter hub to a second drive magnet. The guide catheter hub can be configured to couple to a guide catheter hub adapter by magnetically coupling the guide catheter hub to a third drive magnet. The procedure catheter hub can be configured to couple to a procedure catheter hub adapter by magnetically coupling the procedure catheter hub to a fourth drive magnet. The first drive magnet, the second drive magnet, the third drive magnet, and the fourth drive magnet can be independently movably carried by a drive table. The system can include first driven magnet on the guidewire hub configured to cooperate with the first drive magnet such that the first driven magnet moves in response to movement of the first drive magnet. The first drive magnet can be configured to move outside of a sterile field while separated from the first driven magnet by a sterile field barrier while the first driven magnet is within the sterile field. A position of the first drive magnet can be movable in response to manipulation of a procedure drive control on a control console in electrical communication with the drive table. The system can include a second driven magnet on the access catheter hub configured to cooperate with the second drive magnet such that the second driven magnet is configured to move in response to movement of the second drive magnet, wherein the second drive magnet is configured to move outside of the sterile field while separated from the second driven magnet by the barrier while the second driven magnet is within the sterile field. The system can include a third driven magnet on the guide catheter hub configured to cooperate with the third drive magnet such that the third driven magnet configured to move in response to movement of the third drive magnet, wherein the third drive magnet is configured to move outside of the sterile field while separated from the third driven magnet by the barrier while the third driven magnet is within the sterile field. The system can include a fourth driven magnet on the procedure catheter hub configured to cooperate with the fourth drive magnet such that the fourth driven magnet is configured to move in response to movement of the fourth drive magnet, wherein the fourth drive magnet is configured to move outside of the sterile field while separated from the fourth driven magnet by the barrier while the fourth driven magnet is within the sterile field. The procedure catheter can be an aspiration catheter. The procedure catheter can be an embolic deployment catheter. The procedure catheter can be a stent deployment catheter. The procedure catheter can be a flow diverter deployment catheter. The procedure catheter can be a diagnostic angiographic catheter. The procedure catheter can be a stent retriever catheter. The procedure catheter can be a balloon catheter. The procedure catheter can be a catheter to facilitate percutaneous valve repair or replacement. The procedure catheter can be an ablation catheter. The procedure catheter hub can be configured to adjust a rotational position of the procedure catheter. The access catheter hub can be configured to laterally deflect a distal deflection zone of the access catheter.

There is also provided method of achieving supra-aortic access and neurovascular treatment site access. The method includes the steps of providing a drive system including a guidewire hub configured to adjust an axial position and a rotational position of a guidewire, a procedure catheter hub configured to adjust an axial position and a rotational position of a procedure catheter; a guide catheter hub configured to adjust an axial position of a guide catheter, and an access catheter hub configured to adjust an axial position and a rotational position of an access catheter, the access catheter further configured to laterally deflect a distal deflection zone of the access catheter, and moving at least one of the guidewire hub, the procedure catheter hub, the guide catheter hub, and the access catheter hub to drive movement of at least one of the guidewire, the procedure catheter, the guide catheter, and the access catheter. The method can further include controlling the procedure catheter hub to laterally deflect a distal deflection zone of the procedure catheter.

There is also provided a method of achieving supra aortic access. The method includes the steps of providing an assembly including a guidewire, an access catheter and a guide catheter, coaxially moveably assembled into a single multi-catheter assembly, coupling the assembly to a drive system, driving the assembly to an aortic arch, and advancing the access catheter to achieve supra-aortic access to a branch vessel off of the aortic arch.

The method can further include driving a subset of the assembly to an intracranial site, and performing a neurovascular procedure using the subset of the assembly. The subset can include the guidewire, the guide catheter, and a procedure catheter. The procedure catheter can be an aspiration catheter. The procedure catheter can be an embolic deployment catheter. The procedure catheter can be a stent deployment catheter. The procedure catheter can be a flow diverter deployment catheter. The procedure catheter can be a diagnostic angiographic catheter. The procedure catheter can be a stent retriever catheter. The procedure catheter can be a clot retriever. The procedure catheter can be a balloon catheter. The procedure catheter can be a catheter to facilitate percutaneous valve repair or replacement. The procedure catheter can be an ablation catheter. The intracranial procedure can include an intracranial thrombectomy. The neurovascular procedure can include a neurovascular thrombectomy. At least one of the guidewire, the access catheter, and the guide catheter can include a hub configured to couple to a robotic drive system. Coupling the assembly to the drive system can include magnetically coupling a guide catheter hub to the drive system. Coupling the assembly to the drive system can include mechanically coupling a guide catheter hub to the drive system. The drive system can be a robotic drive system, and at least a first drive magnet, a second drive magnet, and a third drive magnet are each independently movably carried by a drive table associated with the robotic drive system.

There is also provided a robotic drive system. The robotic drive system includes a drive table, a hub adapter coupled with the drive table and configured to move axially along the drive table, and a hub assembly. The hub assembly includes a mount configured to couple to the hub adapter so that axial movement of the hub adapter causes axial movement of the mount, and a hub removably couplable with the mount, the hub being coupled to an interventional device.

The interventional device can be a catheter, wherein the mount is configured to couple to a fluidics management system configured to provide fluid or vacuum to the catheter. The fluidics management system can include a saline tube, a contrast tube, an aspiration tube, and a three-way valve. The hub adapter can further include an active torque subsystem and the mount includes a passive torque subsystem, wherein the active torque subsystem provides an input torque to the passive torque subsystem. The active torque subsystem can further include one or more active torque elements and the passive torque subsystem can further include a corresponding number of passive torque elements, wherein each of the one or more active torque elements are configured to provide an input torque to one of the passive torque elements. The passive torque subsystem can be coupled to the three-way valve. The passive torque subsystem can drive the three-way valve. The three-way valve can be a stopcock. The three-way valve can selectively control flow from the saline tube, the contrast tube, and the aspiration tube to the catheter. The mount can further include a fluidics manifold. The fluidics manifold can further include a plurality of fluidic channels. The fluidics manifold can further include four channels.

There is also provided a hub assembly. The hub assembly includes a mount, a hub removably couplable with the mount, and a fluidics connector in fluid communication with the mount and the hub.

The hub assembly can further include a fluidics management system incorporated within the hub assembly. The fluidics management system can further include a saline tube, a contrast tube, an aspiration tube, and a three-way valve, wherein the three-way valve is in fluid communication with the saline tube, the contrast tube, and the aspiration tube. The hub can further include an interventional device. The interventional device can be in fluid communication with the three-way valve. The three-way valve can be configured to change configurations for providing saline, contrast, and aspiration to the interventional device. The mount can include a passive torque subsystem. The passive torque subsystem can be coupled to the three-way valve. The passive torque subsystem can drive the three-way valve. The three-way valve can be a stopcock. The three-way valve can selectively control flow from the saline tube, the contrast tube, and the aspiration tube to an interventional device. The mount can further include a fluidics manifold. The fluidics manifold further includes a plurality of fluidic channels.

There is also provided a hub assembly. The hub assembly includes a mount being axially movable along a drive surface, a catheter hub having a catheter, the catheter hub removably couplable with the mount, and a fluidics connector in fluid communication with the mount and the catheter hub to provide fluid and vacuum through the connector to the catheter hub.

The mount can be configured to provide saline, contrast, and vacuum through the connector to the catheter hub. The mount can include a saline channel, a contrast channel, and a vacuum channel for providing saline, contrast, and vacuum to the catheter hub. The mount can include a robotically actuated three-way valve coupled to a vacuum channel and a saline-contrast channel, wherein the three-way valve is configured to selectively place the vacuum channel and the saline-contrast channel in fluid communication with the catheter hub. The mount can include a passive torque subsystem including a passive torque element configured to drive the three-way valve. The passive torque element is configured to be actuated by an active torque element of an active torque subsystem separated from the passive torque element by a sterile barrier. The passive torque element includes at least one magnet configured to rotate in response to rotation of at least one magnet of the active torque element. The hub assembly can further include a second robotically actuated three-way valve coupled to the saline channel and the contrast channel and configured to selectively place the saline channel and the contrast channel in fluid communication with the saline-contrast channel. The mount can be configured to transfer motion to the catheter hub to rotate the catheter when the catheter hub is coupled to the mount. The catheter hub can include a gear train coupled to the catheter, wherein the mount includes a gear train configured to couple to the gear train of the catheter hub so that actuation of the gear train of the mount causes rotation of the catheter. The gear train of the catheter hub can include a worm gear. The catheter hub can include a hemostasis valve, wherein the mount is configured to transfer motion to the catheter hub to actuate the hemostasis valve when the catheter hub is coupled to the mount. The catheter hub can include a gear train coupled to the hemostasis valve, wherein the mount includes a gear train configured to couple to the gear train of the catheter hub so that actuation of the gear train of the mount causes actuation of the hemostasis valve. The gear train of the catheter hub can be a first gear train of the catheter hub and the gear train of the mount can be a first gear train of the mount, wherein the catheter hub includes a second gear train coupled to the catheter, wherein the mount includes a second gear train configured to couple to the second gear train of the catheter hub so that actuation of the second gear train of the mount causes rotation of the catheter. The mount can include a manual actuator configured to be actuated by a user to actuate the hemostasis valve. The catheter hub can include a manual actuator configured to be actuated by a user when the catheter hub is uncoupled from the mount to actuate the hemostasis valve. The mount can include a hub receptacle configured to receive the catheter hub. The mount can include one or more electrical connector pins configured to be received within the catheter hub and to establish an electrical circuit to provide electrical and data communication between the mount and the catheter hub. The one or more electrical connector pins can be configured to receive information from the catheter hub regarding a type of the catheter of the catheter hub when the electrical circuit is established. The catheter hub can include a hemostasis valve configured to receive fluid and vacuum from the mount. The catheter hub can include a secondary valve positioned between the hemostasis valve and a proximal end of the catheter, wherein the secondary valve is configured to selectively block fluid communication between the hemostasis valve and a lumen of the catheter. The catheter hub can further include a plunger actuatable to transition a seal of the hemostasis valve between various states. The plunger can be axially movable to a distal position wherein the plunger blocks fluid communication between the hemostasis valve and a lumen of the catheter. The plunger can include one or more channels or slots configured to direct fluid away from the lumen of the catheter when the plunger blocks fluid communication between the hemostasis valve and the lumen of the catheter. The mount can include a sensor positioned to detect a position of the plunger. The catheter hub can include one or more magnets coupled to the plunger, wherein the sensor of the mount includes a hall effect sensor configured to detect the one or more magnets. The hemostasis valve can include a seal, a cap including a plurality of pins, and an auxiliary seal positioned between the seal and the cap and including a plurality of openings configured to receive the plurality of pins of the cap. The mount can include a sensor configured to detect attachment of an anti-buckling device to a proximal end of the catheter hub. The sensor can include a hall sensor configured to detect a magnet on a distal retainer of the anti-buckling device.

There is also provided a hub assembly. The hub assembly includes a mount being axially movable along a drive surface, and an interventional device hub having an interventional device, the interventional device hub being removably couplable with the mount, wherein the mount is configured to transfer motion to the interventional device hub to actuate one or more components of the interventional device hub when the interventional device hub is coupled to the mount.

The mount can be configured to transfer motion to the interventional device hub to rotate the interventional device when the interventional device hub is coupled to the mount. The interventional device hub can include a gear train coupled to the interventional device, wherein the mount includes a gear train configured to couple to the gear train of the interventional device hub so that actuation of the gear train of the mount causes rotation of the interventional device. The gear train of the interventional device hub can include a worm gear. The interventional device hub can include a hemostasis valve, wherein the mount is configured to transfer motion to the interventional device hub to actuate the hemostasis valve when the interventional device hub is coupled to the mount. The interventional device hub can include a gear train coupled to the hemostasis valve, wherein the mount includes a gear train configured to couple to the gear train of the interventional device hub so that actuation of the gear train of the mount causes actuation of the hemostasis valve. The gear train of the interventional device hub can be a first gear train of the interventional device hub and the gear train of the mount can be a first gear train of the mount, wherein the interventional device hub includes a second gear train coupled to the interventional device, wherein the mount includes a second gear train configured to couple to the second gear train of the interventional device hub so that actuation of the second gear train of the mount causes rotation of the interventional device. The mount can include a manual actuator configured to be actuated by a user to actuate the hemostasis valve. The interventional device hub can include a manual actuator configured to be actuated by a user when the interventional device hub is uncoupled from the mount to actuate the hemostasis valve. The mount can include a hub receptacle configured to receive the interventional device hub. The mount can include one or more electrical connector pins configured to be received within the interventional device hub and to establish an electrical circuit to provide electrical and data communication between the mount and the interventional device hub. The one or more electrical connector pins can be configured to receive information from the interventional device hub regarding a type of the interventional device of the interventional device hub when the electrical circuit is established. The interventional device hub can include a hemostasis valve configured to receive fluid and vacuum from the mount. The interventional device hub can include a secondary valve positioned between the hemostasis valve and a proximal end of the interventional device, wherein the secondary valve is configured to selectively block fluid communication between the hemostasis valve and a lumen of the interventional device. The interventional device hub can further include a plunger actuatable to transition a seal of the hemostasis valve between various states. The plunger can be axially movable to a distal position wherein the plunger blocks fluid communication between the hemostasis valve and a lumen of the interventional device. The plunger can include one or more channels or slots configured to direct fluid away from the lumen of the interventional device when the plunger blocks fluid communication between the hemostasis valve and the lumen of the interventional device. The mount can include a sensor positioned to detect a position of the plunger. The interventional device hub can include one or more magnets coupled to the plunger, wherein the sensor of the mount includes a hall effect sensor configured to detect the one or more magnets. The hemostasis valve can include a seal, a cap including a plurality of pins, and an auxiliary seal positioned between the seal and the cap and including a plurality of openings configured to receive the plurality of pins of the cap. The mount can include a sensor configured to detect attachment of an anti-buckling device to a proximal end of the interventional device hub. The sensor can include a hall sensor configured to detect a magnet on a distal retainer of the anti-buckling device.

There is also provided a robotic medical system. The robotic medical system includes a drive table, a hub adapter coupled with the drive table and configured to move axially along the drive table, and a hub assembly. The hub assembly includes a mount configured to couple to the hub adapter so that axial movement of the hub adapter causes axial movement of the mount; and an interventional device hub having an interventional device, the interventional device hub being removably couplable with the mount.

The mount can include an active torque subsystem and the mount includes a passive torque subsystem, wherein the active torque subsystem provides an input torque to the passive torque subsystem. The active torque subsystem can further include one or more active torque elements and the passive torque subsystem can further include a corresponding number of passive torque elements, wherein each of the one or more active torque elements are configured to provide an input torque to one of the one or more passive torque elements. The mount can include a fluid channel, and a vacuum channel for providing fluid and vacuum to the interventional device hub, wherein the mount includes a robotically actuated three-way valve coupled to the fluid channel, wherein the three-way valve is configured to selectively place the vacuum channel and the fluid channel in fluid communication with the interventional device hub. At least one of the one or more passive torque elements can be coupled to the three-way valve and configured to actuate the three-way valve in response to receiving an input torque from one of the one or more active torque elements. The fluid channel includes a saline-contrast channel, wherein the mount further includes a saline channel, a contrast channel, and a second robotically actuated three-way valve coupled to the saline channel and the contrast channel and configured to selectively place the saline channel and the contrast channel in fluid communication with the saline-contrast channel. At least one of the one or more passive torque elements can be coupled to the second three-way valve and configured to actuate the second three-way valve in response to receiving an input torque from one of the one or more active torque elements. The mount can be configured to transfer motion to the interventional device hub to rotate the interventional device when the interventional device hub is coupled to the mount. The interventional device hub can include a gear train coupled to the interventional device, wherein the mount includes a gear train coupled to at least one of the one or more passive torque elements and configured to couple to the gear train of the interventional device hub so that actuation at least one passive torque elements causes rotation of the interventional device. The interventional device hub can include a hemostasis valve, wherein the mount is configured to transfer motion to the interventional device hub to actuate the hemostasis valve when the interventional device is coupled to the mount. The interventional device hub can include a gear train coupled to the hemostasis valve, wherein the mount includes a gear train coupled to at least one of the passive torque elements configured to couple to the gear train of the interventional device hub so that actuation of the at least one of the passive torque elements causes actuation of the hemostasis valve. The mount can include one or more electrical connector pins configured to be received within the interventional device hub and to establish an electrical circuit to provide electrical and data communication between the mount and the interventional device hub. The robotic medical system can further include a control system configured to receive information from the interventional device hub regarding a type of the interventional device of the interventional device hub when the electrical circuit is established.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and a four interventional device assembly.

FIG. 6 is a perspective view of a support table.

FIG. 7 is a close-up view of the motor drive end of a support table.

FIG. 8 is an elevational cross section through a motor and belt drive assembly.

FIG. 9 is a close-up view of a pulley end of the support table.

FIG. 10 is an elevational cross section through a belt pully.

FIG. 11 is a side elevational cross-section through a distal portion of a catheter such as any of those shown in Figures SA and SB.

FIGS. 12A and 12B schematically illustrate a force sensor integrated into the sidewall of the catheter.

FIGS. 13A and 13B schematically illustrate a sensor for measuring elastic forces at the magnetic coupling between the hub and corresponding carriage.

FIG. 14 schematically illustrates a dual encoder torque sensor for use with a catheter of the present disclosure.

FIG. 15 illustrates a clot capture and visualization device that can be integrated into a hub and/or connected to an aspiration line.

FIGS. 16A-16C illustrate an example control mechanism for manipulating interventional devices driven by respective hubs.

FIG. 17 illustrates a side elevational schematic view of an interventional device assembly for supra-aortic access and neuro-interventional procedures.

FIGS. 18A-18E depict an example sequence of steps of introducing a catheter assembly configured to achieve supra-aortic access and neurovascular site access.

FIG. 19 schematically illustrates an embodiment of a mechanical coupling between a drive mechanism and a driven mechanism.

FIGS. 20A-20C depict an example sequence of steps of priming a catheter assembly in a stacked configuration.

FIGS. 21A-21B depict an example sequence of steps of priming a catheter assembly in a stacked configuration.

FIG. 22 depicts an example test system for the priming process depicted in FIGS. 21A-21B.

FIG. 23A shows an example of a catheter assembly.

FIG. 23B shows an example of a catheter assembly after a priming procedure.

FIG. 23C shows an example of a catheter assembly after a priming procedure including relative movement between adjacent catheters.

FIGS. 23D-23F illustrate the example catheter assembly of FIGS. 23A-23C.

FIG. 24 depicts a schematic of a control system.

FIGS. 25A-25B illustrate front and back perspective views of a hub adapter.

FIG. 26 illustrates a front perspective view of a hub assembly.

FIGS. 27A-27B illustrate a perspective view and a back view of a first subassembly, of the hub assembly shown in FIG. 26.

FIGS. 27C-27E illustrate the internal components of a first subassembly shown in FIGS. 27A-27B.

FIGS. 28A-28B illustrate a perspective view and a back view of a second subassembly of the hub assembly shown in FIG. 26.

FIGS. 28C-28G illustrate the internal components of a second subassembly shown in FIGS. 28A-28B.

FIGS. 29A-29B illustrate an alternative embodiment of a first subassembly shown in FIG. 26.

FIGS. 30A-30B illustrate a perspective view of a drive table with a plurality of hub assemblies.

FIG. 31A illustrates a fluidics schematic of a hub assembly.

FIG. 31B illustrates a fluidics flow through a first subassembly.

FIGS. 31C-31D illustrate a fluidics flow through a manifold of a first subassembly.

FIGS. 32A-32E illustrate configurations of a three-way valve for controlling the fluidics of a hub assembly.

FIG. 33A illustrates a top perspective view of an embodiment of a hub coupled to a mount.

FIG. 33B illustrates a bottom view of the mount of FIG. 33A.

FIG. 33C illustrates a front perspective view of a hub decoupled from the mount of FIG. 33A.

FIGS. 33D-33H illustrate views of a mount having a connection pin configured to monitor insertion of a hub within the mount of FIG. 33A.

FIG. 33I illustrates a schematic of a fluidics management system within the mount of FIG. 33A.

FIGS. 33J-33K illustrate components of a hemostasis valve in the hub of FIG. 33A.

FIG. 33L illustrates components of a gear train of the hub of FIG. 33A.

FIGS. 34A-34B illustrate a perspective view and a top view of a forward flush and a backbleed through a hub.

FIG. 34C illustrates a side view of air bubbles within a hemostasis valve of a hub.

FIGS. 34D-34E illustrate a perspective view and a side view of a plunger having one or more longitudinal slots.

FIGS. 34F-34G illustrate a perspective view and a side view of a plunger having one or more radial channels.

FIGS. 34H-34I illustrate valve configurations within a hub.

FIGS. 35A-35D illustrate views of a mount with a sensor configured to monitor the rotational position of a detectable object positioned on a hub.

FIG. 36 illustrates a front view of a mount having a sensor configured to monitor the axial position of a plunger within a hub.

FIG. 37 illustrates a side view of a mount having a sensor configured to monitor an operative state of a locking mechanism.

FIG. 38 illustrates a side view of a mount and a hub having a wireless communication device configured to communicate with a wireless communication device positioned on a hub adapter.

FIG. 39A illustrates a top view of a fluidics management system within a mount having air bubble sensors, rotational position sensors, and a pressure sensor.

FIGS. 39B-39C illustrate a perspective view and a side view of a three-way valve having a detectable object configured to be monitored by a sensor.

FIG. 40 illustrates a perspective view of a mount having one or more manual actuation buttons and status indicators.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table can be positioned over or alongside the patient, and configured to axially advance, retract, and in some cases rotate and/or laterally deflect two or three or more different (e.g., concentrically or side by side oriented) intravascular devices. The hub is moveable along a path along the surface of the drive table to advance or retract the interventional device as desired. Each hub may also contain mechanisms to rotate or deflect the device as desired, and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. Each hub is releasably magnetically coupled to a unique drive carriage on the table side of the sterile field barrier. The drive system independently moves each hub in a proximal or distal direction across the surface of the barrier, to move the corresponding interventional device proximally or distally within the patient's vasculature.

The carriages on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

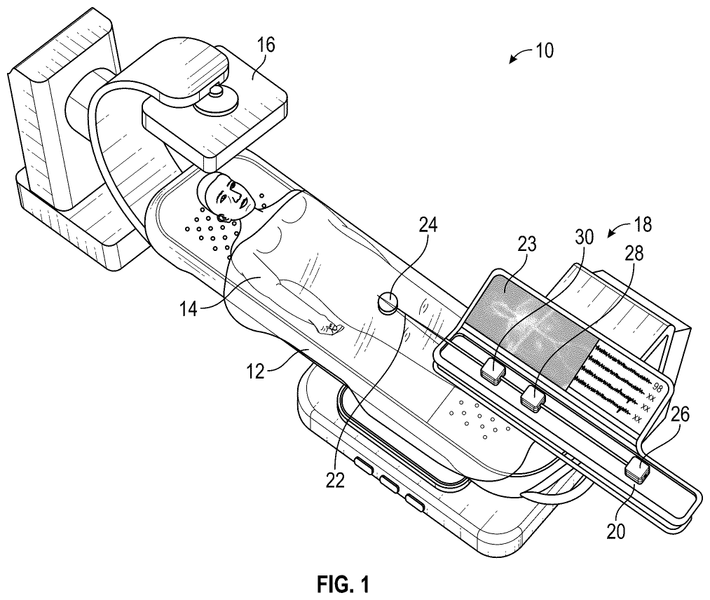

FIG. 1 is a schematic perspective view of an interventional setup 10 having a patient support table 12 for supporting a patient 14. An imaging system 16 may be provided, along with a robotic interventional device drive system 18 in accordance with the present disclosure.

The drive system 18 may include a support table 20 for supporting, for example, a guidewire hub 26, an access catheter hub 28 and a guide catheter hub 30. In the present context, the term ‘access’ catheter can be any catheter having a lumen with at least one distally facing or laterally facing distal opening, that may be utilized to aspirate thrombus, provide access for an additional device to be advanced therethrough or therealong, or to inject saline or contrast media or therapeutic agents.

More or fewer interventional device hubs may be provided depending upon the desired clinical procedure. For example, in certain embodiments, a diagnostic angiogram procedure may be performed using only a guidewire hub 26 and an access catheter hub 28 for driving a guidewire and an access catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23 such as for viewing fluoroscopic images, catheter data (e.g., fiber Bragg grating fiber optics sensor data or other force or shape sensing data) or other patient data may be carried by the support table 20 and or patient support 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control, and may also be configured to laterally deflect a deflectable portion of the access catheter, in response to manipulation of a deflection control.

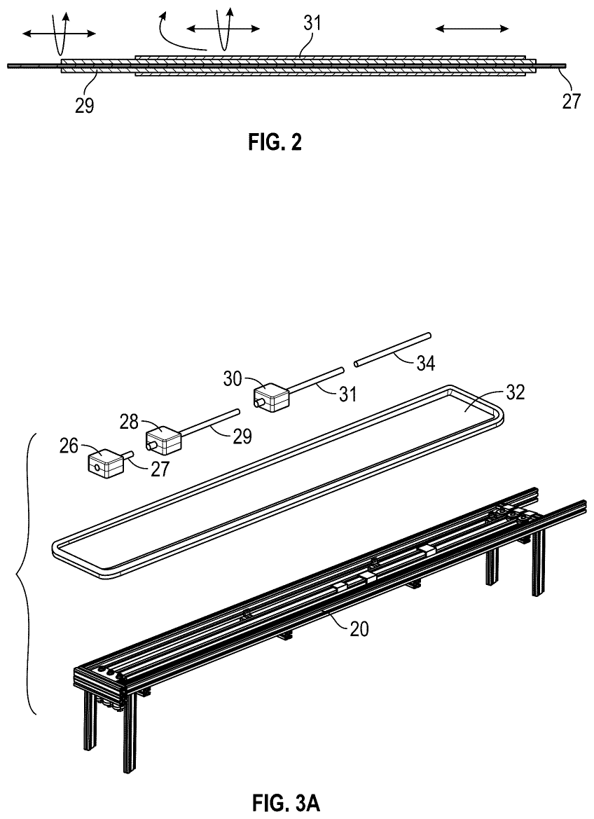

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

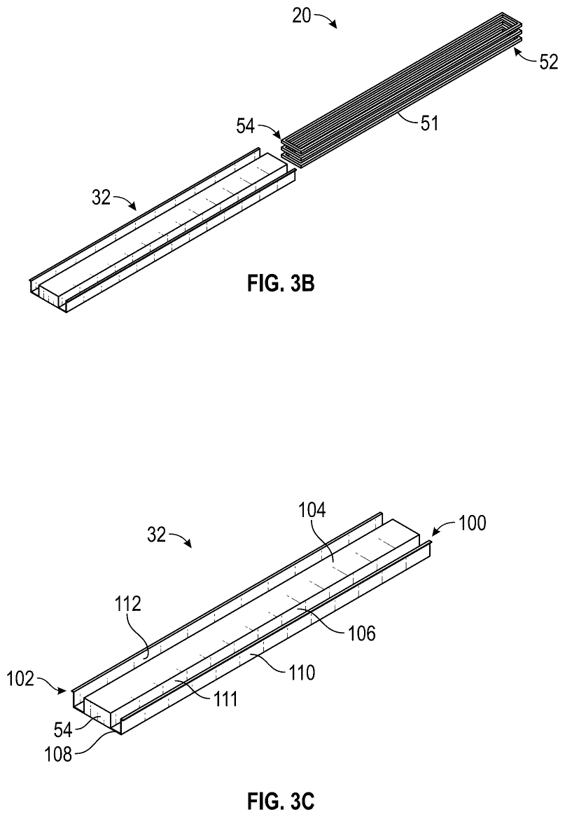

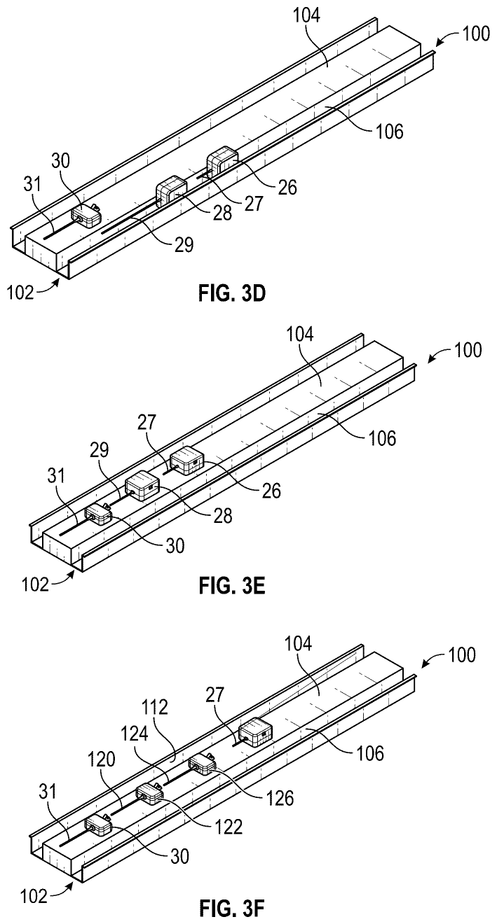

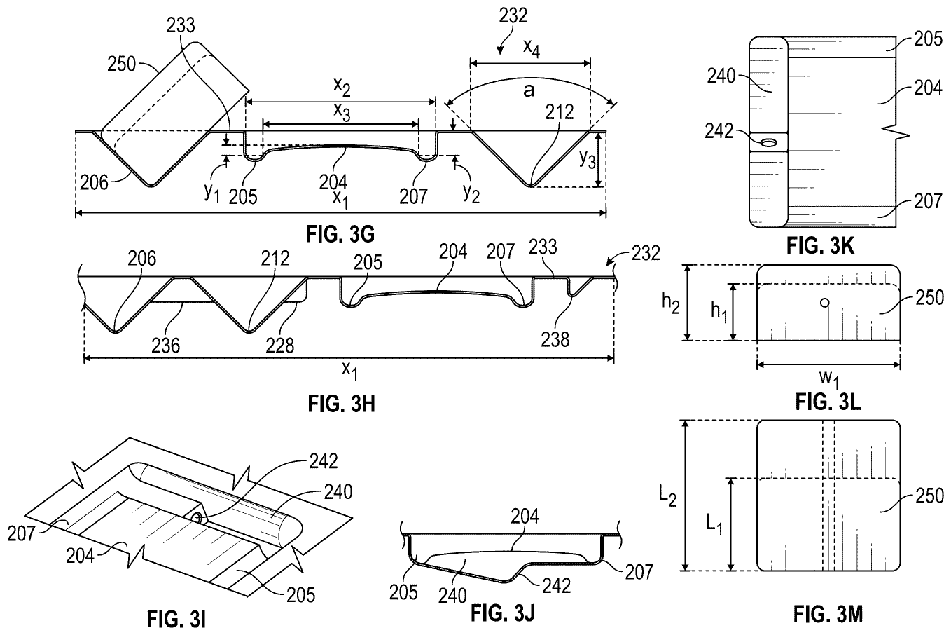

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The antibuckling feature 34 may include a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypo tube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may include a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), or styrene. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 would be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 will typically be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guidewire hub 26 and guidewire 27 are illustrated residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices configured in an access assembly for aortic access, following coupling of the access assembly to the corresponding carriages beneath the sterile barrier. The access assembly may be preassembled with the guidewire fully advanced through the access catheter which is in turn fully advanced through the guide catheter. In embodiments in which the access catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guidewire hub 26 is positioned most proximally, in order to allow the guidewire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

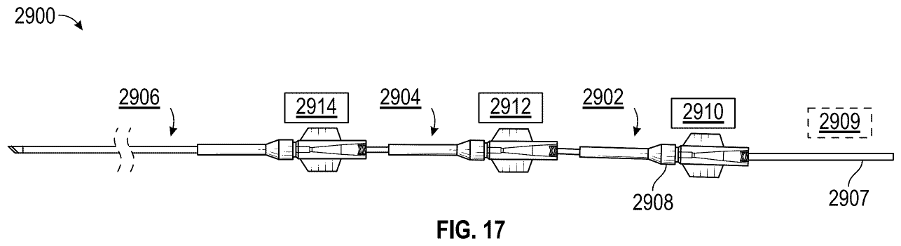

As is discussed in greater detail in connection with FIG. 17, the multi catheter stack may be utilized to achieve both access and the intravascular procedure without the need for catheter exchange. this may be accomplished in either a manual or a robotically driven procedure. In one example, the guide catheter 31 may include a catheter having an inner diameter of at least about 0.08 inches and in one implementation about 0.088 inches. The first procedure catheter 120 may include a catheter having an inner diameter within the range of from about 0.065 inches to about 0.075 inches and in one implementation catheter 120 has an inner diameter of about 0.071 inches. The second procedure catheter 124 may be an access catheter having an OD sized to permit advance through the first procedure catheter 120. The second procedure catheter maybe steerable, having a deflection control 2908 configured to laterally deflect a distal end of the catheter. The second procedure (access) catheter may also have an inner lumen sized to allow an appropriately sized guidewire to remain inside the second procedure catheter while performing contrast injections through the second procedure catheter.

In certain embodiments, the catheter 31 may be a ‘large bore’ access catheter or guide catheter having a diameter of at least about 0.075 or at least about 0.080 inches in diameter. The catheter 120 may be an aspiration catheter having a diameter within the range of from about 0.060 to about 0.075 inches. The catheter 124 may be a steerable catheter with a deflectable distal tip, having a diameter within the range of from about 0.025 to about 0.050 inches. The guidewire 27 may have a diameter within the range of from about 0.014 to about 0.020 inches. In one example, the catheter 31 may have a diameter of about 0.088 inches, the catheter 120 about 0.071 inches, the catheter 124 about 0.035 inches, and the guidewire 27 may have a diameter of about 0.018 inches.

In one commercial execution, a preassembled access assembly (guide catheter, access catheter and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure.

FIGS. 3G-3K illustrate embodiments of an alternate sterile barrier having a convex drive surface (e.g., a convex, crowned road like drive surface). FIG. 3G is a cross sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

FIGS. 3I, 3J, and 3K illustrate a sectional perspective view, a cross-sectional view, and a top sectional view, respectively, of a proximal end of the sterile barrier 232. As shown, in FIGS. 3I-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 3I-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width X3 of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w₁ of 3.75 in, about 3.75 in, between 3.25 in and 4.25 in, or any other suitable width. The hub 250 can have a height h₁ of 1.5 in, about 1.5 in, between 1.25 in and 1.75 in, or any other suitable height. Alternatively, the hub 250 can have a height h₂ of 2 in, about 2 in, between 1.75 in and 2.25 in, or any other suitable height. In some embodiments, the hub 250 can have a length L₁ of 2.5 in, about 2.5 in, between 2 in and 3 in or any other suitable length. Alternatively, the hub 250 can have a length L₂ of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead in the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in of FIGS. 3A-3F). A vertical configuration may provide for easier interaction with the drive system 18 by a physician, A vertical configuration may also provide for a lower axis of catheter travel closer to a patient without adding standoff height to the drive system 18.

In some embodiments, the drive system 18 may be positioned, during part of or the entirety of an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's feet.

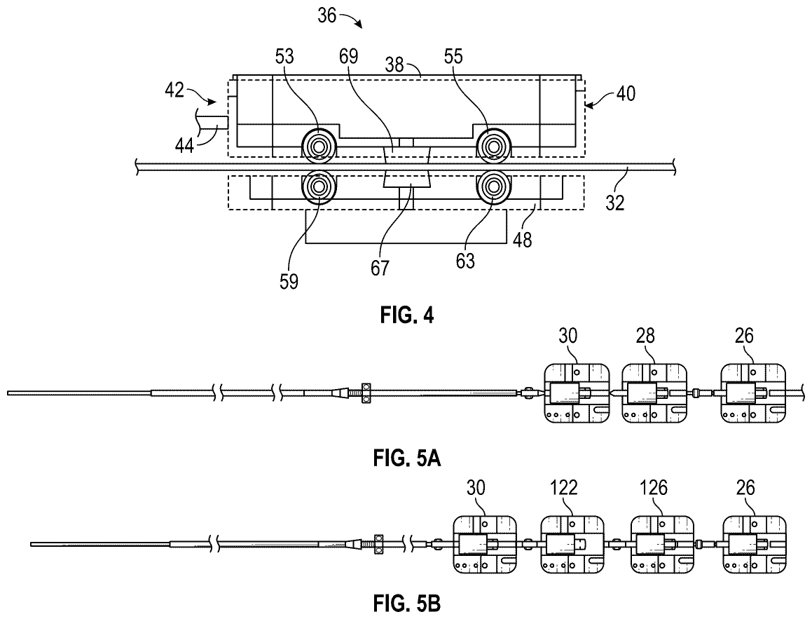

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions or controller manipulations. The hub adapter 48 includes at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) and about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4.

Referring to FIG. 6, there is schematically illustrated one example of a low-profile linear drive support table 20. Support table 20 includes an elongated frame 51 extending between a proximal end 52 and a distal end 54. At least one support table support 56 is provided to stabilize the support table 20 with respect to the patient (not illustrated). Support 56 may include one or more legs or preferably an articulating arm configured to allow movement and positioning of the frame 51 over or adjacent to the patient.

One example of a linear drive table 20 illustrated in FIG. 7 includes three distinct drives. However, two drives or four or more drives (e.g., up to eight drives) may be included depending upon the desired clinical performance. A first drive pulley 58 engages a first drive belt 60. A first carriage bracket 61 is secured to the first drive belt 60 such that rotation of the first drive pulley 58 causes rotation of the first drive belt 60 through an elongate closed loop path. The first carriage bracket 61 may be advanced in a proximal or distal direction along the longitudinal axis of the support table 20 depending upon the direction of rotation of the drive pully 58. In the illustrated implementation, the drive pulley 58 is provided with surface structures such as a plurality of drive pulley teeth 62 for engaging complementary teeth on the first drive belt 60.

A second drive pulley 64 may engage a second drive belt 66 configured to axially move a second carriage bracket 68 along an axial path on the support table 20. A third drive pulley 70 may be configured to drive a third drive belt 72, to advance a third carriage bracket 73 axially along the support table 20. Each of the carriage brackets may be provided with a drive magnet assembly discussed previously but not illustrated in FIG. 7, to form couplers for magnetically coupling to a corresponding driven magnet within the hub of an interventional device as has been discussed.

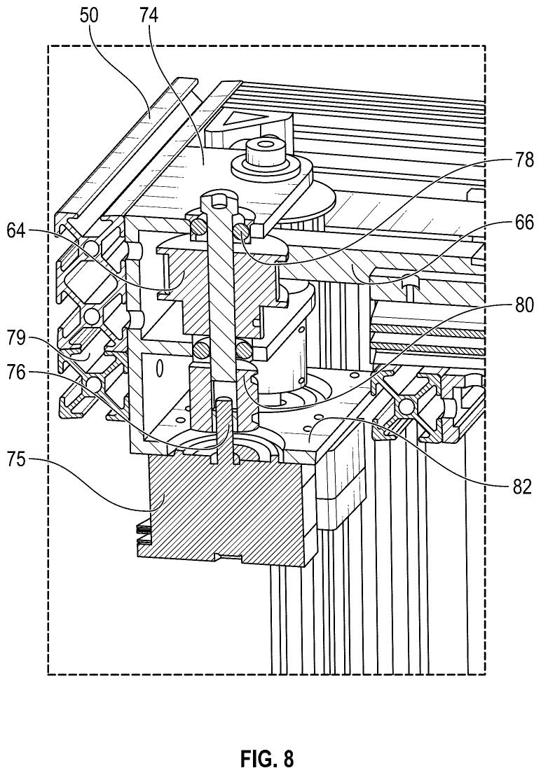

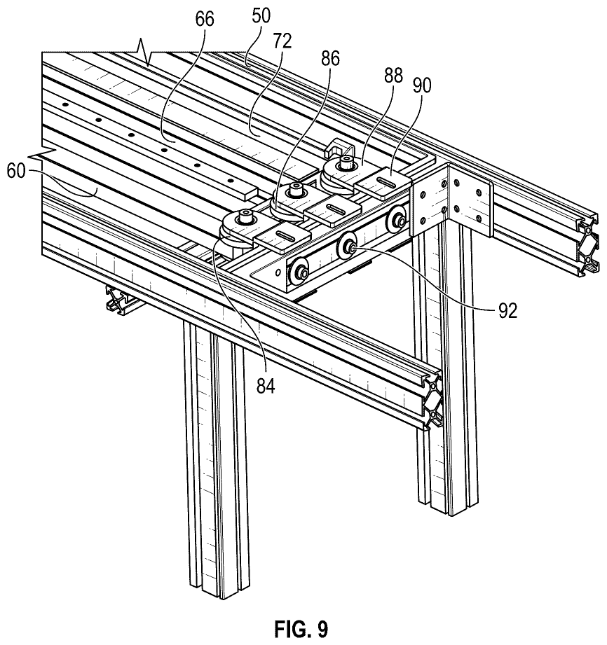

A detailed view of a drive system is shown schematically in FIG. 8. A drive support 74 may be carried by the frame 51 for supporting the drive assembly. The second drive pulley 64 is shown in elevational cross section as rotationally driven by a motor 75 via a rotatable shaft 76. The rotatable shaft 76 may be rotatably carried by the support 74 via a first bearing 78, a shaft coupling 80 and second bearing 79. Motor 75 may be stabilized by a motor bracket 82 connected to the drive support 74 and or the frame 51. The belt drive assemblies for the first drive belt 60 and third drive belt 72 maybe similarly constructed and are not further detailed herein. In some embodiments, the drive systems described herein may be a rack and pinion drive table system that is foldable. In such embodiments, motors 75 may be attached to and move with the carriages.

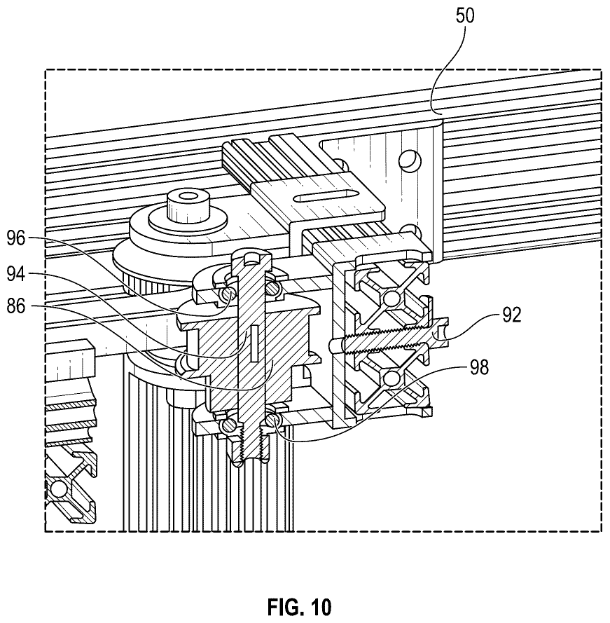

Referring to FIGS. 9 and 10, each of the first second and third drive belts extends around a corresponding first idler pulley 84 second idler pulley 86 and third idler pulley 88. Each idler pulley may be provided with a corresponding tensioning bracket 90, configured to adjust the idler pulleys in a proximal or distal direction in order to adjust the tension of the respective belt. Each tensioning bracket 90 is therefore provided with a tensioning adjustment 92 such as a rotatable screw.

As seen in FIG. 10, the second idler pulley 86, for example, may be carried by a rotatable shaft 94, rotatably secured with respect to the mounting bracket by a first bearing 96 and second bearing 98.

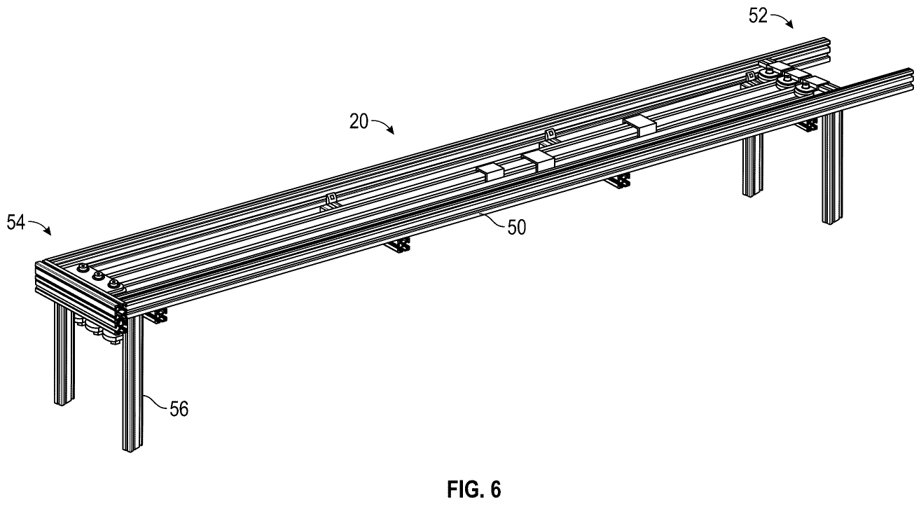

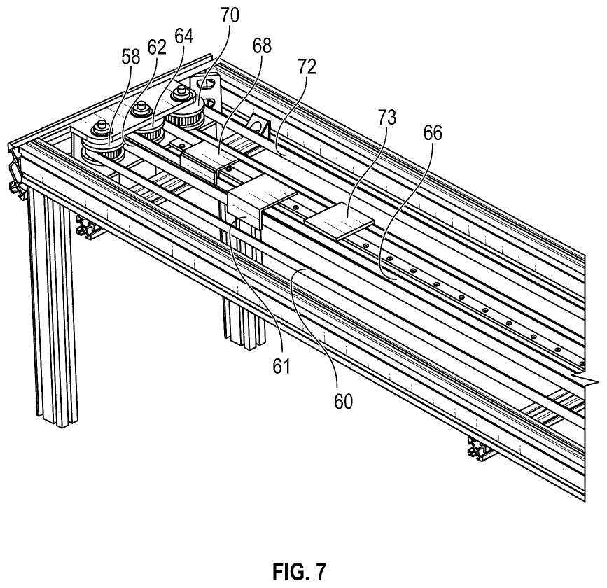

Any of the catheters illustrated, for example, in FIG. 5A, 5B or 11 generally include an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

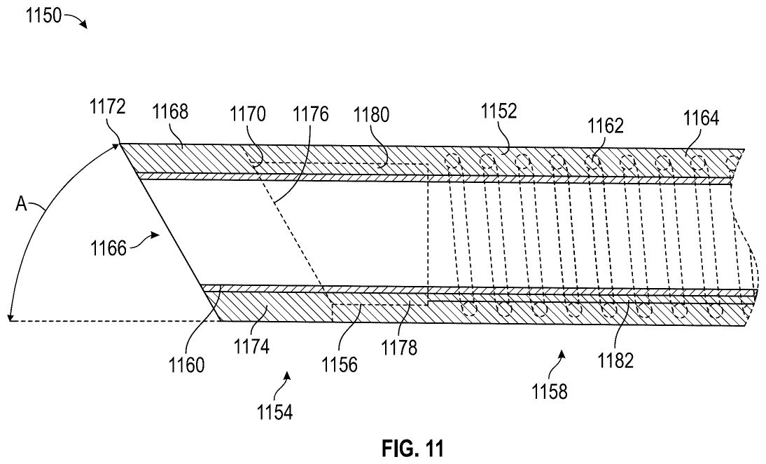

Any of the catheters disclosed herein may be provided with an inclined distal tip. Referring to FIG. 11, distal catheter tip 1150 includes a tubular body 1152 which includes an advance segment 1154, a marker band 1156 and a proximal segment 1158. An inner tubular liner 1160 may extend throughout the length of the distal catheter tip 1150, and may include dip coated or extruded PTFE or other lubricious material.

A reinforcing element 1162 such as a braid and/or spring coil is embedded in an outer jacket 1164 which may extend the entire length of the catheter.

The advance segment 1154 terminates distally in an angled face 1166, to provide a leading side wall portion 1168 having a length measured between the distal end 130 of the marker band 1156 and a distal tip 1172. In some embodiments, the entire distal tip may be shaped to avoid snagging the tip in areas of arterial bifurcation. A trailing side wall portion 1174 of the advance segment 1154, has an axial length in the illustrated embodiment of approximately equal to the axial length of the leading side wall portion 1168 as measured at approximately 180 degrees around the catheter from the leading side wall portion 1168. The leading side wall portion 1168 may have an axial length within the range of from about 0.1 millimeters to about 5 millimeters and generally within the range of from about 1 to 3 millimeters. The trailing side wall portion 1174 may be equal to or at least about 0.1 or 0.5 or 1 millimeter or 2 millimeters or more shorter than the axial length of the leading side wall portion 1168, depending upon the desired performance.

The angled face 1166 inclines at an angle A within the range of from about 45 degrees to about 80 degrees from the longitudinal axis of the catheter. For certain implementations, the angle is within the range of from about 55 degrees to about 65 degrees from the longitudinal axis of the catheter. In one implementation, the angle A is about 60 degrees. One consequence of an angle A of less than 90 degrees is an elongation of a major axis of the area of the distal port which increases the surface area of the port and may enhance clot aspiration or retention. Compared to the surface area of the circular port (angle A is 90 degrees), the area of the angled port is generally at least about 105 percent, and no more than about 130 percent, in some implementations within the range of from about 110 percent and about 125 percent, and in one example is about 115 percent of the area of the corresponding circular port (angle A is 90 degrees).

In the illustrated embodiment, the axial length of the advance segment is substantially constant around the circumference of the catheter, so that the angled face 1166 is approximately parallel to the distal surface 1176 of the marker band 1156. The marker band 1156 has a proximal surface approximately transverse to the longitudinal axis of the catheter, producing a marker band 1156 having a right trapezoid configuration inside elevational view. A short sidewall 1178 is rotationally aligned with the trailing side wall portion 1174, and has an axial length within the range of from about 0.2 millimeters to about 4 millimeters, and typically from about 0.5 millimeters to about 2 millimeters. An opposing long sidewall 1180 is rotationally aligned with the leading side wall portion 1168. Long sidewall 1180 of the marker band 1156 is generally at least about 10 percent or 20 percent longer than short sidewall 1178 and may be at least about 50 percent or 70 percent or 90 percent or more longer than short sidewall 1178, depending upon desired performance. Generally, the long sidewall 1180 will have a length of at least about 0.5 millimeters or 1 millimeter and less than about 5 millimeters or 4 millimeters.

The marker band may be a continuous annular structure, or may have at least one and optionally two or three or more axially extending slits throughout its length. The slit may be located on the short sidewall 1178 or the long sidewall 1180 or in between, depending upon desired bending characteristics. The marker band may include any of a variety of radiopaque materials, such as a platinum/iridium alloy, with a wall thickness preferably no more than about 0.003 inches and in one implementation is about 0.001 inches.

The fluoroscopic appearance of the marker bands may be unique or distinct for each catheter size or type when a plurality of catheters is utilized so that the marker bands can be distinguishable from one another by a software algorithm. Distinguishing the marker bands of a plurality of catheters may be advantageous when the multiple catheters are used together, for example, in a multi catheter assembly or stack as described herein. In some embodiments, the marker band of a catheter may be configured so that a software algorithm can detect motion of the catheter tip.

The marker band zone of the assembled catheter may have a relatively high bending stiffness and high crush strength, such as at least about 50 percent or at least about 100 percent less than proximal segment 18 but generally no more than about 200 percent less than proximal segment 1158. The high crush strength may provide radial support to the adjacent advance segment 1154 and particularly to the leading side wall portion 1168, to facilitate the functioning of distal tip 1172 as an atraumatic bumper during transluminal advance and to resist collapse under vacuum. The proximal segment 1158 preferably has a lower bending stiffness than the marker band zone, and the advance segment 1154 preferably has even a lower bending stiffness and crush strength than the proximal segment 1158.

The advance segment 1154 may include a distal extension of the outer tubular jacket 1164 and optionally the inner liner 1160, without other internal supporting structures distally of the marker band 1156. Outer jacket 1164 may include extruded polyurethane, such as Tecothane. The advance segment 1154 may have a bending stiffness and radial crush stiffness that is no more than about 50 percent, and in some implementations no more than about 25 percent or 15 percent or 5 percent or less than the corresponding value for the proximal segment 1158.

The catheter may further include an axial tension element or support such as a ribbon or one or more filaments or fibers for increasing the tension resistance and/or influencing the bending characteristics in the distal zone. The tension support may include one or more axially extending mono strand or multi strand filaments. The one or more tension element 1182 may be axially placed inside the catheter wall near the distal end of the catheter. The one or more tension element 1182 may serve as a tension support and resist tip detachment or elongation of the catheter wall under tension (e.g., when the catheter is being proximally retracted through a kinked outer catheter or tortuous or narrowed vasculature).

At least one of the one or more tension element 1182 may proximally extend along the length of the catheter wall from within about 1.0 centimeters from the distal end of the catheter to less than about 10 centimeters from the distal end of the catheter, less than about 20 centimeters from the distal end of the catheter, less than about 30 centimeters from the distal end of the catheter, less than about 40 centimeters from the distal end of the catheter, or less than about 50 centimeters from the distal end of the catheter.

The one or more tension element 1182 may have a length greater than or equal to about 40 centimeters, greater than or equal to about 30 centimeters, greater than or equal to about 20 centimeters, greater than or equal to about 10 centimeters, or greater than or equal to about 5 centimeters.

At least one of the one or more tension element 1182 may extend at least about the most distal 50 centimeters of the length of the catheter, at least about the most distal 40 centimeters of the length of the catheter, at least about the most distal 30 centimeters or 20 centimeters or 10 centimeters of the length of the catheter.

In some implementations, the tension element extends proximally from the distal end of the catheter along the length of the coil 24 and ends proximally within about 5 centimeters or 2 centimeters or less either side of a transition between a distal coil and a proximal braid. The tension element may end at the transition without overlapping with the braid.

The one or more tension element 1182 may be placed near or radially outside the inner liner 1160. The one or more tension element 1182 may be placed near or radially inside the braid and/or the coil. The one or more tension element 1182 may be carried between the inner liner 1160 and the helical coil, and may be secured to the inner liner or other underlying surface by an adhesive prior to addition of the next outer adjacent layer such as the coil. Preferably, the tension element 1182 is secured to the marker band 1156 such as by adhesives or by mechanical interference. In one implementation, the tension element 1182 extends distally beyond the marker band on a first (e.g., inside) surface of the marker band, then wraps around the distal end of the marker band and extends along a second (e.g., outside) surface in either or both a proximal inclined or circumferential direction to wrap completely around the marker band.

When more than one tension element 1182 or filament bundles are spaced circumferentially apart in the catheter wall, the tension elements 1182 may be placed in a radially symmetrical manner. For example, the angle between two tension elements 1182 with respect to the radial center of the catheter may be about 180 degrees. Alternatively, depending on desired clinical performances (e.g., flexibility, trackability), the tension elements 1182 may be placed in a radially asymmetrical manner. The angle between any two tension elements 1182 with respect to the radial center of the catheter may be less than about 180 degrees, less than or equal to about 165 degrees, less than or equal to about 135 degrees, less than or equal to about 120 degrees, less than or equal to about 90 degrees, less than or equal to about 45 degrees or, less than or equal to about 15 degrees.

The one or more tension element 1182 may comprise materials such as Vectran®, Kevlar®, Polyester®, Spectra®, Dyneema®, Meta-Para-Aramide®, or any combinations thereof. At least one of the one or more tension element 1182 may include a single fiber or a multi-fiber bundle, and the fiber or bundle may have a round or rectangular (e.g., ribbon) cross section. The terms fiber or filament do not convey composition, and they may include any of a variety of high tensile strength polymers, metals or alloys depending upon design considerations such as the desired tensile failure limit and wall thickness. The cross-sectional dimension of the one or more tension element 1182, as measured in the radial direction, may be no more than about 2 percent, 5 percent, 8 percent, 15 percent, or 20 percent of that of the catheter 10.

The cross-sectional dimension of the one or more tension element 1182, as measured in the radial direction, may be no more than about 0.03 millimeters (about 0.001 inches), no more than about 0.0508 millimeters (about 0.002 inches), no more than about 0.1 millimeters (about 0.004 inches), no more than about 0.15 millimeters (about 0.006 inches), no more than about 0.2 millimeters (about 0.008 inches), or about 0.38 millimeters (about 0.015 inches).

The one or more tension element 1182 may increase the tensile strength of the distal zone of the catheter before failure under tension (e.g., marker band detachment) to at least about 1 pound, at least about 2 pounds, at least about 3 pounds, at least about 4 pounds, at least about 5 pounds, at least about 6 pounds, at least about 7 pounds, at least about 8 pounds, or at least about 10 pounds or more.

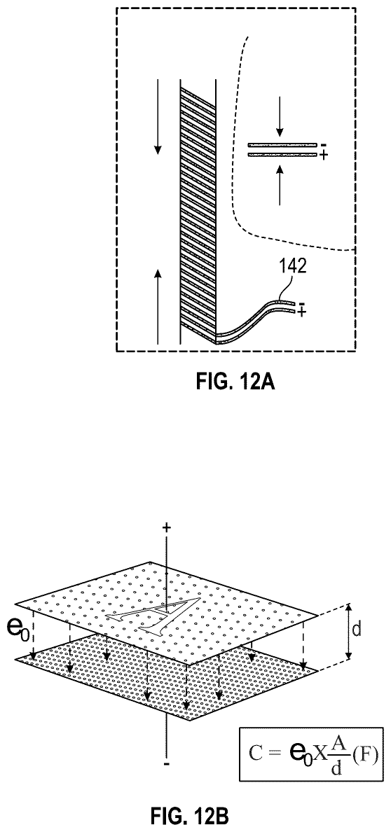

Any of a variety of sensors may be provided on any of the catheters, hubs, carriages, or table, depending upon the desired data. For example, in some implementations, it may be desirable to measure axial tension or compression force applied to the catheter such as along a force sensing zone. The distal end of the catheter would be built with a similar construction as illustrated in FIG. 11, with a helical coil distal section. But instead of using a single helical coil of nitinol wire, a first conductor 140 and second conductor 142 are wrapped into intertwined helical coils and electrically isolated from each other such as by the plastic/resin of the tubular body. See FIG. 12A. Each coil is in electrical communication with the proximal hub by a unique electrical conductor such as a conductive trace or proximal extension of the wire.

This construction of double, electrically isolated helical coils creates a capacitor. This is roughly equivalent to two plates of nitinol with a plastic layer between them, illustrated in FIG. 12B. The capacitance is inversely proportional to the distance between wires. The only variable that would be changing would be d, the distance between the plates. If an axial compressive force is applied to the catheter, the wires (e.g., conductor 140 and conductor 142) will move closer together, thus increasing the capacitance. If an axial tensile force is applied, the wires will get further apart, decreasing the capacitance. This capacitance can be measured at the proximal end of the catheter, giving a measurement of the force at the helical capacitor. Although referred to as a capacitor, this sensor is measuring the electrical interaction between the two coils of wire. There may be a measurable change in inductance or other resulting change due to applied axial forces.

At least a first helical capacitor may have at least one or five or ten or more complete revolutions of each wire. A capacitor may be located within the distal most 5 or 10 or 20 centimeters of the catheter body to sense forces experienced at the distal end. At least a second capacitor may be provided within the proximal most 5 or 10 or 20 centimeters of the catheter body, to sense forces experienced at the proximal end of the catheter.

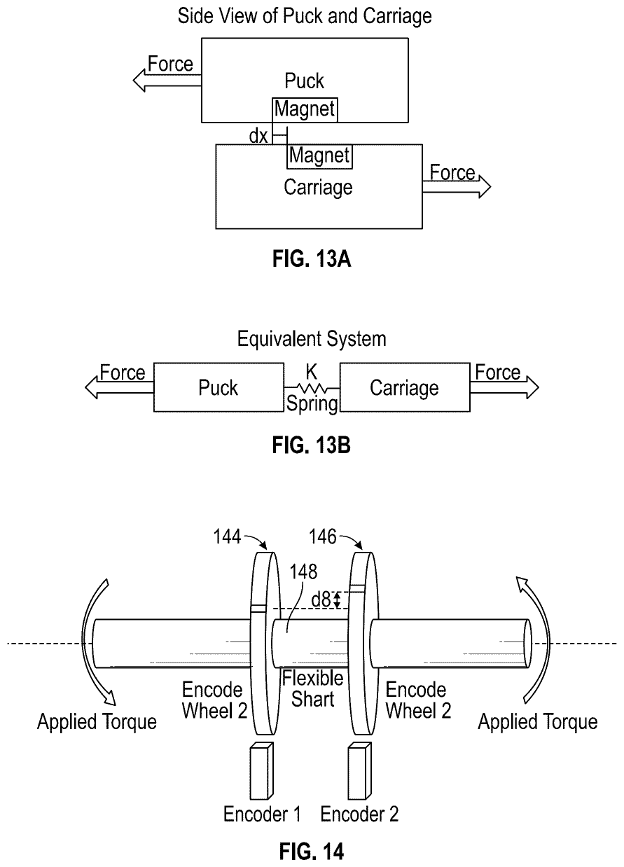

It may also be desirable to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub will move a small amount relative to the carriage. See FIG. 13A. In robotics, this is called a series clastic actuator. This property can be used to measure the force applied from the carriage to the hub. To measure the force, the relative distance between the hub and the carriage (dx shown in FIG. 13A) is determined and characterize some effective spring constant k between the two components. See FIG. 13B.

The relative distance could be measured in multiple different ways. One method for measuring the relative distance between the hub and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magnetoresistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors would preferably be configured in a manner that avoids accumulation of blood or other fluid in the interface between the hubs carriages. In some implementations, wireless (i.e., inductive) power may be used to translate movement and/or transfer information across the sterile barrier between a drive carriage and a hub, for example.

The magnetic coupling between the hub and the carriage has a shear or axial break away threshold which may be about 300 grams or 1000 grams or more. The processor can be configured to compare the axial force applied to the catheter to a preset axial trigger force which if applied to the catheter is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and or torque in the vicinity of the sensor.

It may also be desirable to understand the three-dimensional configuration of the catheter or guidewire during and/or following transvascular placement. Shape sensing fiber optics such as an array of FBG fibers to sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque. Such as at the distal tip and/or proximal end of the device.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the hubs through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. Comparing distal tip position or movement or lack thereof to commanded or actual proximal catheter or guidewire movement at the hub, may be used to detect a loss of relative motion, which may be indicative of a device shaft buckling, prolapse, kinking, or a similar outcome (for example, along the device shaft length inside the body (e.g., in the aorta) or outside the body between hubs. The processing may be done in real time to provide position/orientation data at up to 30 Hertz, although this technique would only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy.

Proximal torque applied to the catheter or guidewire shaft may be determined using a dual encoder torque sensor. Referring to FIG. 14, a first encoder 144 and a second encoder 146 may be spaced axially apart along the shaft 148, for measuring the difference in angle over a length of flexible catheter/tube. The difference in angle is interpolated as a torque, since the catheter/tube has a known torsional stiffness. As torque is applied to the shaft, the slightly flexible portion of the shaft will twist. The difference between the angles measured by the encoders (dθ) tells us the torque. T=k*dθ, where k is the torsional stiffness.

Confirming the absence of bubbles in fluid lines may also be accomplished using bubble sensors, particularly where the physician is remote from the patient. This may be accomplished using a non-contact ultrasonic sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

Preferably, a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path. In other embodiments, the bubble removal system can include a pump and control system upstream of the bubble detector for removal of in line bubbles. A processor may be configured to activate the pump upon detection of bubbles to reverse the fluid flow and clear the bubbles into a waste reservoir before reestablishing bubble free forward flow.

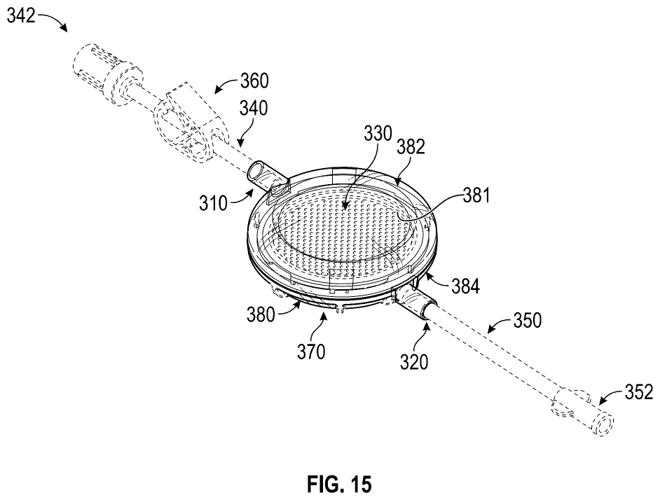

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump. Referring to FIG. 15, one example of a clot retrieval device 370 can include a body 380 enclosing a chamber 381 which communicates with a first port 310 and a second port 320.

In some embodiments, the body 380 includes a housing having a top portion 382 and a bottom portion 384. The body 380 may include a filter 330 positioned in the chamber 381 between the top portion 382, and the bottom portion 384. In some examples, the first port 310 is configured to connect to a first end of a first tube 340 that is fluidly connected to a proximal end of an aspiration catheter.

In an embodiment that is configured to be connected downstream from the hub, the first tube 340 includes a connector 342 positioned at a second end of the first tube 340 that is configured to engage or mate with a corresponding connector on or in communication with the hub. The first port 310 directly communicates with the chamber on the upstream (e.g., top side) of the filter, and the second port 320 directly communicates with the chamber on the downstream (e.g., bottom side) of the filter to facilitate direct visualization of material caught on the upstream side of the filter.

In an implementation configured for remote operation, any of a variety of sensors may be provided to detect clot passing through the aspiration line and/or trapped in the filter, such as an optical sensor, pressure sensor, flow rate sensor, ultrasound sensor or others known in the art.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector on the pump.

In some examples, the system 300 can include an on-off valve 360 such as a clamp. The clamp can be positioned in between the filter 330 and the patient, such as over the first tube 340 to allow the user to engage the clamp and provide flow control by isolating the patient from the clot retrieval device 370. Closing the valve 360 and operating the remote vacuum pump (not illustrated) causes the canister associated with the vacuum pump and the chamber 381 to reach the same low pressure. Due to the short distance and small line volume of the lumen between the chamber 381 end the distal end of the catheter, a sharp negative pressure spike is experienced at the distal end of the catheter rapidly following opening of the valve 360. Additional details are disclosed in U.S. Pat. No. 11,259,821 issued Mar. 1, 2022 to Buck et al., entitled Aspiration System with Accelerated Response, the entirety of which is hereby expressly incorporated by reference herein. In some embodiments, a vacuum may be cycled against a clot to retrieve the clot. The vacuum may be automatically and robotically controlled to remove the clot.

The body 380 can have a top surface spaced apart from a bottom surface by a tubular side wall. In the illustrated implementation, the top and bottom surfaces are substantially circular, and spaced apart by a cylindrical side wall. The top surface may have a diameter that is at least about three times, or five times or more than the axial length (transverse to the top and bottom surfaces) of the side wall, to produce a generally disc shaped housing. Preferably at least a portion of the top wall is optically transparent to improve clot visualization once it is trapped in the clot retrieval device 370. Additional details may be found in U.S. patent application Ser. No. 18/436,882, entitled Device For Clot Retrieval With Varying Tube Diameters, filed Feb. 8, 2024, the entirety of which is hereby expressly incorporated by reference herein.

In some examples, the body 380 can include a flush port (not illustrated) that is configured to allow the injection of an optically transparent media such as air, saline or other fluid into the chamber 381 to clear an optical path between the window and the filter to improve clot visualization once it is trapped in the filter 330.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, entitled Catheter Drive System For Supra-Aortic Access, filed Nov. 16, 2021, the entirety of which is hereby expressly incorporated by reference herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

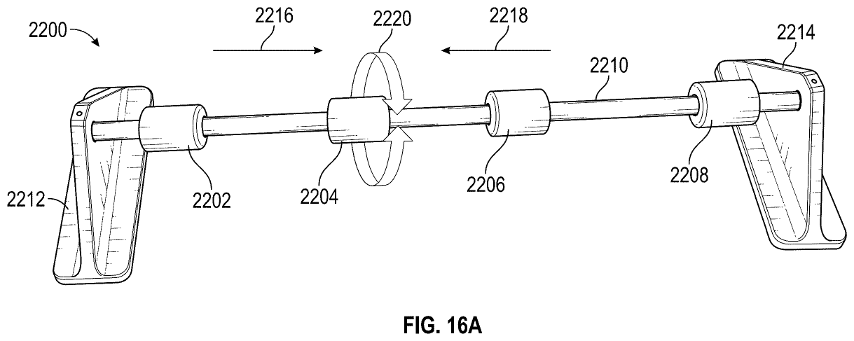

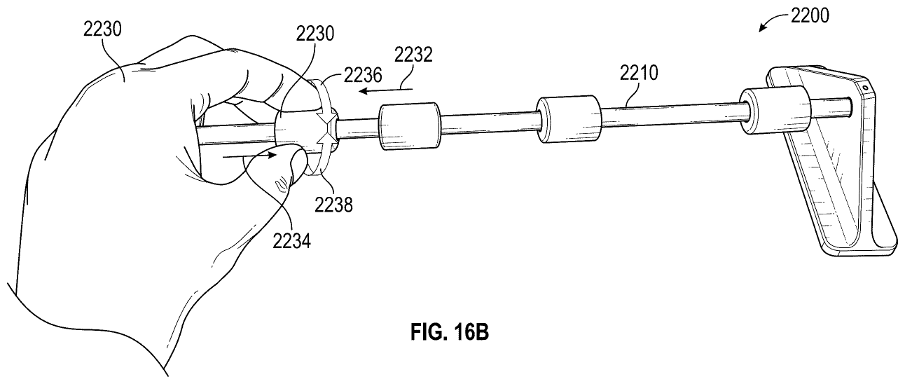

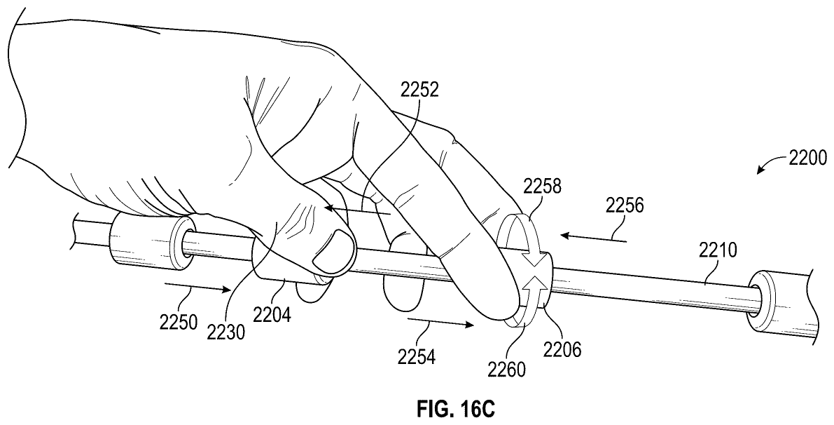

FIGS. 16A-16C illustrate an example control mechanism 2200 for manipulating interventional devices driven by (or otherwise associated with) respective hubs. For example, each hub may be manipulated and/or otherwise moved using at least one control installed in control mechanism 2200. Each control may be adapted to move a unique hub and associated interventional device during an interventional procedure.

As shown in FIG. 16A, the control mechanism 2200 include a first control 2202, a second control 2204, a third control 2206, and a fourth control 2208. More or fewer controls may be provided, depending upon the intended interventional devices configuration. Each control 2202-2208 is movably carried on a shaft 2210 that is coupled to a distal bracket 2212 and to a proximal bracket 2214. The controls 2202-2208 may advance distally or retract proximally on the shaft 2210, as indicated by arrow 2218 and arrow 2216. In addition, each control 2202-2208 may also be rotated about the shaft 2210, as indicated by arrow 2220. Each control movement may trigger a responsive movement in a corresponding carriage on the support table, which may in turn drive movement of a corresponding hub as has been discussed.

The control mechanism 2200 may be positioned on or near to a patient support table having a set of hubs and catheters/interventional devices. In some implementations, the control mechanism 2200 may be positioned remote from the support table such as behind a radiation shield or in a different room or different geographical location in a telemedicine implementation.

Each control 2202-2208 may correspond to and drive movement of a hub and/or a hub and interventional device combination. For example, the control 2202 may be configured to drive hub 30 (FIG. 3F) to move an interventional device such as an 0.088 inch guide catheter corresponding to the hub 30. Similarly, the control 2204 may be configured to drive hub 28 (122) to move an interventional device such as an 0.071 inch procedure catheter. The control 2206 may be configured to drive hub 126 to move an interventional device such as a steerable access catheter. The control 2208 may be configured to drive hub 26 to axially and rotationally move an interventional device such as a guidewire.

FIG. 16B illustrates an example of manually manipulating the control 2202 on control mechanism 2200. In operation, if the user 2230 moves the control 2202 axially along shaft 2210 and distally, as shown by arrow 2232, a corresponding coupled hub and/or interventional device may move responsively in the same direction by a same or scaled amount. If the user 2230 rotates the control 2202 about the shaft 2210 and advances the control proximally, as shown by arrow 2234, a corresponding coupled interventional device will responsively move rotationally and proximally by a same or scaled amount. If the user 2230 moves the control 2202 rotationally about the shaft 2210, as shown by arrow 2236 or arrow 2238, a corresponding coupled hub will drive the corresponding interventional device rotationally in the same direction and/or by a same or scaled amount.

Other axes and degrees of freedom may be defined to enable control 2202 to perform movements that may be translated to movement of hubs and/or interventional devices. For example, the control mechanism may be provided with one or more deflection controls configured to initiate a lateral deflection in a deflection zone on the corresponding interventional device.

Axial movement of a control may be configured to move the coupled hub on a 1:1 basis, or on a non 1:1 scaled basis. For example, if the user 2230 advances the control 2022 about 5 millimeters distally along the shaft 2210, then the corresponding hub may responsively move 5 millimeters in the distal direction.

If the user 2230 rotates the control 2022 about its rotational axis by 5 degrees, the coupled hub will cause the corresponding interventional device to rotate on a 1:1 basis or on a non 1:1 scaled basis. The scaled amount may be selected to reduce or increase the amount of distance and rotation that a hub and/or interventional device moves in accordance with the control movement.

In some implementations, the scaled amount described herein may be determined using a scale factor. The scale factor may apply to one or both translational and rotational movement. In some implementations, a first scale factor is selected for translational movement and a second scale factor, different than the first scale factor, is selected for rotational movement. The axial scaling factor may drive proximal catheter movement at a faster speed than distal catheter movement for a given proximal or distal manipulation of the control.

The rotational scale factor may be 1:1 while the axial scale factor may move the hub by a greater distance than movement of the control such that hub travel to control travel is at least about 2:1 or 5:1 or 10:1 or more depending upon the desired axial length of the control assembly.

The control mechanism 2200 may be configured to enable the clinician to adjust the scale factor for different parts of the procedure. For example, distal advance of the procedure catheter and access catheter through the guide catheter and up to the selected ostium may desirably be accomplished in a ‘fast’ mode. But more distal travel into the neuro vasculature may desirably be accomplished in a relatively slow mode by actuation of a speed control.

In another implementation, one or more controls may be configured to progressively drive advance or retraction speeds of the corresponding hub and associated catheter. For example, distal control 2202 may drive the guide catheter. A slight distal movement of the control 2202 may advance the guide catheter distally at a slow speed, while advancing the control 2202 by a greater distance distally increases the rate of distal travel of the guide catheter.

Controlling the speed of the corresponding hubs either axially or both axially and rotationally may enhance the overall speed of the procedure. For example, advance of the various devices from the femoral access point up to the aortic arch may desirably be accomplished at a faster rate than more distal navigation closer to the treatment site. Also proximal retraction of the various devices, particularly the guidewire, access catheter and procedure catheter may be desirably accomplished at a relatively higher speeds than distal advance.

FIG. 16C illustrates another example of manually manipulating a control on the control mechanism 2200 to move hubs and/or other interventional devices. In some implementations, two or more controls 2202-2208 may be moved in combination to trigger movement of one or more hubs and/or related interventional devices. In the depicted example, the user 2230 moves control 2204 and control 2206 in combination (e.g., sequentially, simultaneously) such as to simultaneously move the 0.088 guide catheter and the 0.071 aspiration catheter as a unit. Example movement of control 2204 may include axial proximal movement in the directions shown by arrows 2250. Sequentially or simultaneously, the user 2230 may move control 2206 axially in either of the directions shown by arrows 2254 and 2256 while also moving control 2206 rotationally in either of the directions shown by arrows 2258 and 2260.

In some implementations, each control mechanism and/or additional controls (not shown) may be color coded, shaped coded, tactile coded, or other coding to indicate to the user 2230 which color is configured to move which hub or interventional device. In some implementations, the control color coding may also be applied to the hubs and/or interventional devices such that a user may visually match a particular hub/device with a particular control.

In some implementations, other control operations beyond translational movement and rotational movement may be carried out using controls 2202-2208. For example, controls 2202-2208 may be configured to drive a shape change and/or stiffness change of a corresponding interventional device. Controls 2202-2208 may be toggled between different operating modes. For example, controls 2202-2208 may be toggled between movement driven by acceleration and velocity to movement that reflects actual linear displacement or rotation.

In some implementations, the control mechanism 2200 may be provided with a visual display or other indicator of the relative positions of the controls which may correspond the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

In some implementations, the control mechanism 2200 may include haptic components to provide haptic feedback to a user operating the controls. For example, if the control 2202 is triggering movement of a catheter and the catheter detects a large force at the tip, the control 2202 may generate haptic feedback to indicate to the user to stop or reverse a performed movement. In some implementations, haptic feedback may be generated at the control to indicate to the user to slow or speed a movement using the control. In some implementations, haptics may provide feedback on a large torsional strain buildup that might precede an abrupt rotation, or a large axial force buildup that may be a prelude to buckling of the catheter.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

FIG. 17 illustrates a side elevational schematic view of a multi catheter interventional device assembly 2900 for combined supra-aortic access and/or neurovascular site access and procedure (e.g., aspiration), as described herein. The multi catheter assembly 2900 may be configured for either a manual or a robotic procedure.

The interventional device assembly 2900 includes an insert or access catheter 2902, a procedure catheter 2904, and a guide catheter 2906. Other components are possible including, but not limited to, one or more guidewires (e.g., optional guide wire 2907), one or more guide catheters, an access sheath and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-catheter assembly 2900 may be used without having to exchange hub components. For example, in the two stage procedure disclosed previously, a first stage for achieving supra-aortic access includes mounting an access catheter, guide catheter and guidewire to the support table. Upon gaining supra aortic access, the access catheter and guidewire were typically removed from the guide catheter. Then, a second catheter assembly is introduced through the guide catheter after attaching a new guidewire hub and a procedure catheter hub to the corresponding drive carriage on the support table.

The single multi catheter assembly 2900 of FIG. 17 is configured to be operated without having to remove hubs and catheters and without the addition of additional assemblies and/or hubs. Thus, the multicomponent access and procedure configuration of assembly 2900 may utilize a guidewire 2907 manufactured to function as an access guidewire and a navigation guidewire to allow for sufficient access and support, and navigation to the particular distal treatment site. In a non-limiting example configured for robotic implementation, a catheter assembly may include a guidewire hub (e.g., guidewire hub 2909 or guidewire hub 26 positioned on a drive table and to the right of catheter 2902), an insert or access catheter hub 2910, a procedure catheter hub 2912, a guide catheter hub 2914 and corresponding catheters. In certain embodiments, one or more of the hubs may include or be coupled to a hemostasis valve (e.g., a rotating hemostasis valve) to accommodate introduction of interventional devices therethrough. Additional details regarding hemostasis valves and fluidics systems are included in U.S. patent application Ser. No. 17/879,614, entitled Multi Catheter System With Integrated Fluidics Management, filed Aug. 2, 2022, and U.S. patent application Ser. No. 18/666,217, entitled Fluidics Control System For Multi Catheter Stack, filed May 16, 2024, each of which is hereby expressly incorporated by reference in its entirety herein.

Once access above the aortic arch has been achieved, the insert or access catheter 2902 (associated with insert or access catheter hub 2910) may be parked in the vicinity of a carotid artery ostia and the remainder or a subset of the catheter assembly may be guided more distally toward a particular site (e.g., a clot site, a surgical site, a procedure site, etc.).

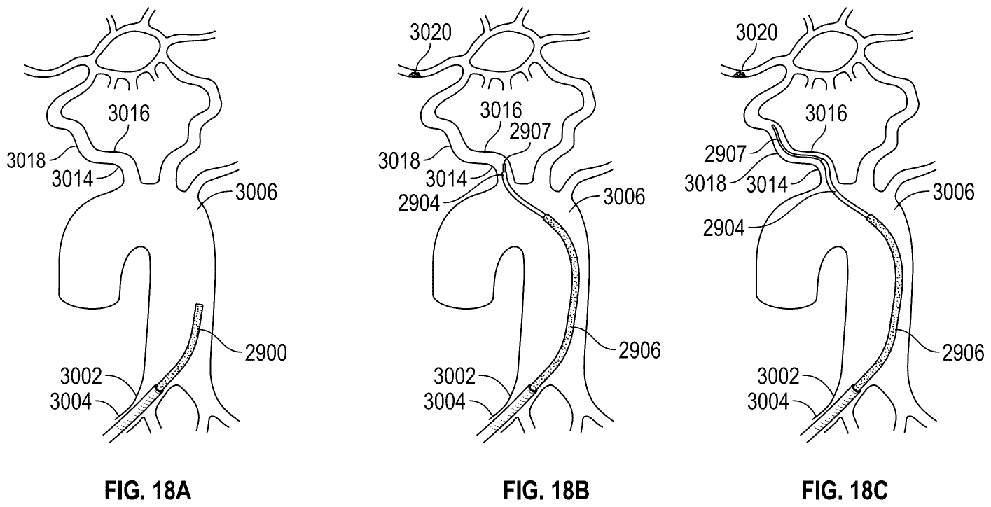

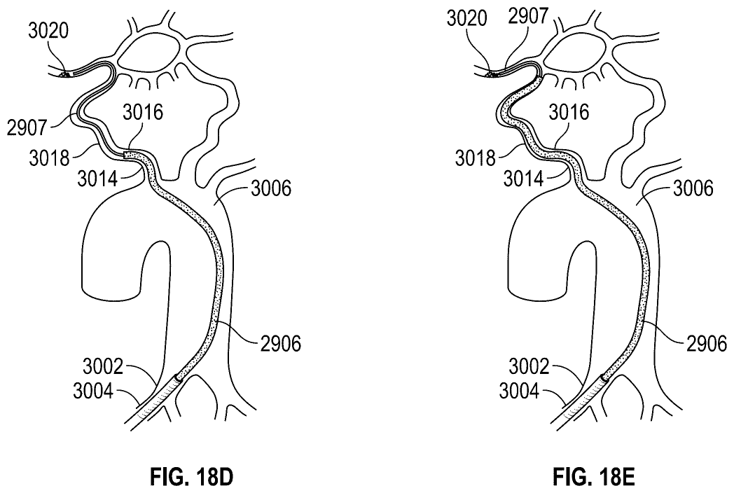

In some embodiments, other smaller procedure catheters may also be added and used at the site. As used herein for catheter assembly 2900, in a robotic configuration of assembly 2900, the guide catheter 2906 may function as a guide catheter. The procedure catheter 2904 may function as a procedure (e.g., aspiration) catheter. In some embodiments, the guide catheter 2906 may function to perform aspiration in addition to functioning as a guide catheter, either instead of or in addition to the procedure catheter 2904. The access catheter 2902 may have a distal deflection zone and can function to access a desired ostium. One of skill in the art will appreciate from FIGS. 18A-18E that either manual manipulation or robotic manipulation of the multi catheter stack are contemplated herein.

In some embodiments, the catheter assembly 2900 (or other combined catheter assemblies described herein) may be driven as a unit to a location. However, each catheter (or guidewire) component may instead be operated and driven independent of one another to the same or different locations.

In a non-limiting example, the catheter assembly 2900 may be used for a diagnostic angiogram procedure. In some embodiments, the assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

As shown in FIG. 17, the guide catheter 2906, procedure catheter 2904, access catheter 2902, and guidewire 2907 can be arranged concentrically. In certain embodiments, the guide catheter 2906 may be a ‘large bore’ guide catheter or access catheter having a diameter of at least about 0.075 or at least about 0.080 inches in diameter. The procedure catheter 2904 may be an aspiration catheter having a diameter within the range of from about 0.060 to about 0.075 inches. The access catheter 2902 may be a steerable catheter with a deflectable distal tip, having a diameter within the range of from about 0.025 to about 0.050 inches. The guidewire 2907 may have a diameter within the range of from about 0.014 to about 0.020 inches. In one example, the guide catheter 2906 may have a diameter of about 0.088 inches, the procedure catheter 2904 about 0.071 inches, the access catheter 2902 about 0.035 inches, and the guidewire 2907 may have a diameter of about 0.018 inches.

FIGS. 18A-18E depict an example sequence of steps of introducing a multicatheter assembly configured to achieve access all the way to the clot, either manually or robotically. FIGS. 18A-18E may be described using the interventional device assembly of FIG. 17. Other combinations of catheters may be substituted for the interventional device assembly, as will be appreciated by those of skill in the art in view of the disclosure herein.

Referring to FIG. 18A, the three catheter interventional device assembly 2900 is shown driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 18B. Here, the distal end of the guide catheter 2906 may be parked below the aortic arch 3006 and the procedure catheter 2904, access catheter 2902 (positioned within the procedure catheter 2904 and not visible in FIG. 18B), and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure access. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 18B, the procedure catheter 2904 has been advanced into the ostium while the guide catheter 2906 has remained parked below the aortic arch 3006.

Referring to FIG. 18C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 18D, the guide catheter 2906 and the procedure catheter 2904 (positioned within the guide catheter 2906 and not visible in FIG. 18D) are both advanced (e.g., simultaneously or sequentially) over the guidewire 2907 and over the insert or access catheter 2902 (positioned within the procedure catheter 2904 and not visible in FIG. 18D) while the access catheter 2902 remains at the ostium for support. The guidewire 2907 may be further advanced past the petrous segment 3018 to the site of the clot 3020, such as the Ml segment.

Referring to FIG. 18E, the guide catheter 2906 and the procedure catheter 2904 (positioned within the guide catheter 2906 and not visible in FIG. 18E) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 18E) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procure catheter 2904 may be removed before removing the guide catheter 2906.

The catheter assembly 2900 may be used to perform a neurovascular procedure, as described in FIGS. 18A-18E. For example, the neurovascular procedure may be a neurovascular thrombectomy. The steps of the procedure may include providing an assembly that includes at least a guidewire, an access catheter, a guide catheter, and a procedure catheter. For example, the catheter assembly 2900 includes a guidewire 2907, an access (e.g., insert) catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter, a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system, and driving the assembly to achieve supra-aortic access. The steps may further include driving a subset of the assembly to a neurovascular site, and performing the neurovascular procedure using a subset of the assembly. The subset of the assembly may include the guidewire, the guide catheter, and the procedure catheter.

Each of the guidewire 2907, the access catheter 2902, the guide catheter 2906, and the procedure catheter 2904 is configured to be adjusted by a respective hub. For example, the guidewire 2907 may include (or be coupled to) a hub installed on one of the tray assemblies described herein. Similarly, the access catheter 2902 may be coupled to insert or access catheter hub 2910. The guide catheter 2906 may be coupled to the guide catheter hub 2914. The procedure catheter 2904 may be coupled to the procedure catheter hub 2912.

In general coupling of the assembly may include magnetically coupling a first hub such as a guidewire hub 2909 on the guidewire 2907 to a first drive magnet, magnetically coupling a second hub such as an insert or access catheter hub 2910 on the access catheter 2902 to a second drive magnet, magnetically coupling a third hub such as a procedure catheter hub 2912 on the procedure catheter 2904 to a third drive magnet, and magnetically coupling a fourth hub such as a guide catheter hub 2914 on the guide catheter 2906 to a fourth drive magnet. In general, the first drive magnet, the second drive magnet, the third drive magnet, and the fourth drive magnet are each independently movably carried by a drive table, as described with respect to tray assemblies and controls described herein. In some embodiments, the first drive magnet, the second drive magnet, the third drive magnet, and the fourth drive magnet are coupled (e.g., to their respective catheter hubs) through a sterile barrier (e.g., a sterile and fluid barrier) and independently movably carried by a drive table having a plurality of driven magnets. In some embodiments, two or more drive magnets can be tethered or otherwise coupled together to move as a unit in response to commands from a single controller tethered or otherwise coupled to one of the drive magnets.

In some implementations, the steps of performing the neurovascular procedure may include driving the assembly in response to movement of each of the hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a driven magnet carried by the respective hub. This provides a magnetic coupling between the drive magnet and driven magnet through the sterile barrier such that the respective hub is moved across the top of the sterile barrier in response to movement of the hub adapter outside of the sterile field (as described in detail in FIG. 4). Movement of the hub adapter is driven by a drive system carried by the support table in which the guidewire hub 2909, the guide catheter hub 2914, the procedure catheter hub 2912, and the insert or access catheter hub 2910 are installed upon.

The steps may further include driving a subset of the assembly in response to movement of each of the hub adapters along the support table until the subset of the assembly is positioned to perform a neurovascular procedure at a neurovascular treatment site. The subset of the assembly may include the guidewire 2907, the guide catheter 2906, and the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the catheter assembly 2900 may be part of a robotic control system for achieving supra-aortic access and neurovascular treatment site access, as described in FIGS. 18A-18E. In some embodiments, the catheter assembly 2900 may be part of a manual control system for achieving supra-aortic access and neurovascular treatment site access. In some embodiments, the catheter assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an insert or access catheter hub 2910 configured to adjust axial and rotational movement of an access catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect a distal deflection zone of the procedure catheter 2904. In some embodiments, the guidewire hub 2909 is configured to couple to a guidewire hub adapter by magnetically coupling the guidewire hub to a first drive magnet. The insert or access catheter hub 2910 is configured to couple to an access catheter hub adapter by magnetically coupling the insert or access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail with respect to FIG. 4 above.

In some embodiments, the robotic control system includes a second driven magnet on the insert or access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the access catheter 2902, the procedure catheter 2904, and/or the guide catheter 2906) and/or one or more hubs (e.g., the guidewire hub 2909, the insert or access catheter hub 2910, the procedure catheter hub 2912, and/or the guide catheter hub 2914) with one or more drive mechanisms.

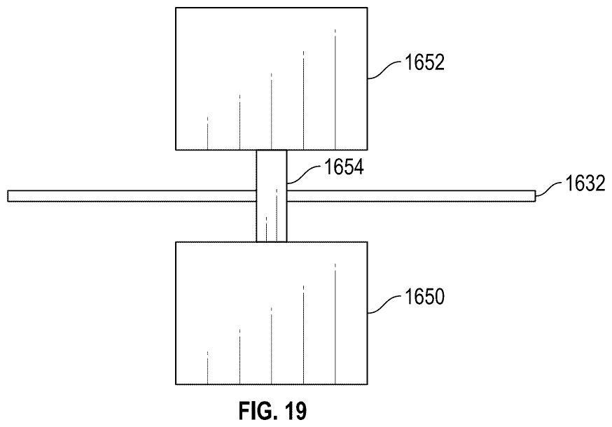

FIG. 19 illustrates a mechanical coupling mechanism 1654 between a drive mechanism 1650 and a driven mechanism 1652. Drive mechanism 1650 and driven mechanism 1652 may have any of the same or similar features or functions as the drive magnet 67 and driven magnet 69, respectively, except as otherwise described herein. The drive mechanism 1650 may be part of or coupled to a hub adapter (e.g., the hub adapter 48). The driven mechanism 1652 may be part of or coupled to a hub (e.g., the hub 36, the guidewire hub 2909, the insert or access catheter hub 2910, the procedure catheter hub 2912, or the guide catheter hub 2914). In some instances, the mechanical coupling mechanism 1654 may include a structural support (e.g., a support rod or support strut) extending transversely through a seal in a sterile barrier 1632. The seal may permit the structural support to be advanced along a length of the sterile barrier 1632, while still maintaining a seal with the structural support to maintain the sterile field, as the drive mechanism 1650 and driven mechanism 1652 are advanced and/or retracted as described herein. For example, the seal may include a tongue and groove closure mechanism along the sterile barrier 1632 that is configured to close on either side of the structural support while permitting passage of the structural support through the sterile barrier 1632 and maintaining a seal against the structural support as the structural support is advanced along the length of the sterile barrier 1632.

In some embodiments, the structural support can extend through an elongate self closing seal between two adjacent coaptive edges of flexible material (e.g., similar in shape to a duckbill valve) that extends along an axis. As the structural support advances along the axis between the coaptive edges, the coaptive edges may permit the structural support to advance, and then may be biased back into a sealing engagement with each other as the structural support passes any given point along the axis.

In some embodiments, the drive mechanism may be a splined drive shaft (e.g., a non-sterile splined drive shaft). The mechanical coupling mechanism 1654 can include a pulley within a plate that serves as the sterile barrier 1632 and a sterile splined shaft configured to couple to the driven mechanism 1652. The driven mechanism 1652 can be a sterile pulley that receives the sterile splined shaft from the sterile barrier. In some embodiments, one or more splined drive shafts can engage and turn corresponding pulleys in the plate that serves as the sterile barrier. Each hub can have a sterile pulley that is configured to receive a sterile splined shaft from the sterile barrier plate. Rotation of the splined drive shaft can turn the pulley in the sterile barrier plate which can in turn turn the sterile pulley in the hub via the sterile splined shaft.

It will be understood by one having skill in the art that any embodiment as described herein may be modified to incorporate a mechanical coupling mechanism, for example, as shown in FIG. 19.

The interventional devices described herein may be provided individually or at least some of the interventional devices can be provided in a preassembled (e.g., nested or stacked) configuration. For example, the interventional devices may be provided in the form of an interventional device assembly, such as interventional device assembly 2900, in a concentric nested or stacked configuration. If provided individually, each catheter (and in some embodiments, each corresponding catheter hub) can be unpackaged and primed to remove air from its inner lumen, for example, by flushing the catheter (and in some embodiments, the corresponding catheter hub) to remove air by displacing it with a fluid, such as saline, contrast media, or a mixture of saline and contrast media. After priming, the interventional devices can be manually assembled into a stacked configuration so that they are ready for introduction into the body for a surgical procedure, for example, via an introducer sheath.

Assembling the devices into a stacked configuration can include individually inserting interventional devices into one another by order of size. For example, an interventional device having a second largest diameter can be inserted into the lumen of an interventional device having a largest diameter. An interventional device having a third largest diameter can then be inserted into the interventional device having the second largest diameter and so on.

For example, with respect to FIG. 17, assembly can be performed by first inserting a distal end of the procedure catheter 2904 through the guide catheter hub 2914 and into the guide catheter 2906. The procedure catheter 2904 can be advanced through the guide catheter 2906 until the distal tip of the procedure catheter 2904 is flush with or extends beyond the distal tip of the guide catheter 2906, and/or until the procedure catheter 2904 cannot be inserted any further. Then, the distal end of the catheter 2902 can be inserted through the procedure catheter hub 2912 and into the procedure catheter 2904. The catheter 2902 can be advanced through the procedure catheter 2904 until the distal tip of the catheter 2902 is flush with or extends beyond the distal tip of the procedure catheter 2904, and/or until the catheter 2902 cannot be inserted any further. Then, the distal end of the guidewire 2907 can be inserted through the insert or access catheter hub 2910 and into the catheter 2902. The guidewire 2907 can be advanced through the catheter 2902 until the distal tip of the guidewire 2907 is flush with or extends beyond the distal tip of the catheter 2902, and/or until the guidewire 2907 cannot be inserted any further.

Embodiments in which two or more of the interventional devices are packaged together as a single unit in an assembled (e.g., nested or stacked) configuration may provide efficient unpackaging and preparation prior to use and efficient assembly within a robotic control system. The interventional devices may be pre-mounted to their respective hubs prior to packaging. In certain embodiments, two or three or more interventional devices may be packaged in a fully nested (i.e., fully axially inserted) configuration or nearly fully nested configuration. In a fully nested configuration, each interventional device is inserted as far as possible into an adjacent distal hub and interventional device. Such a fully nested configuration may minimize a total length of the interventional device assembly and minimize the size of the packaging required to house the interventional device assembly.

In some embodiments, the interventional devices may also be sterilized prior to packaging while in the assembled configuration, for example, using ethylene oxide gas. In some embodiments, the interventional devices may be packaged while in the assembled configuration before sterilization with ethylene oxide gas. For interventional devices in a nested or stacked configuration, ethylene oxide gas can be provided in a space between adjacent interventional devices (for example, an annular lumen between an outer diameter of a first interventional device nested within a second interventional device and the inner diameter of the second interventional device) for sterilization. In some embodiments, the interventional device assembly can be packaged in a thermoformed tray and sealed with an HDPE (e.g., Tyvek®) lid. The interventional device assembly can be unpackaged by removal (e.g., opening or peeling off) of the lid by a user in a non-sterile field. A user in the sterile field can then remove the interventional device assembly and place it on the sterile work surface, for example, of a robotic drive table, as described herein.

Packaging the interventional devices in an assembled configuration and sterilized state can reduce the time associated with unpackaging and assembly of individual interventional devices and facilitate efficient connection to a robotic drive system. Each interventional device and hub combination may further be packaged with a fluidics connection for coupling to a fluid source and/or a vacuum source. In some embodiments, each hub or a hemostasis valve coupled to the hub may include the fluidics connection.

After the interventional device assembly is unpackaged (e.g., after the interventional device assembly is positioned on the robotic drive table), priming can be performed while the devices are concentrically nested or stacked. This is preferably accomplished in each fluid lumen, such as, for example, the annular lumen between the guide catheter 2906 and the procedure catheter 2904 and in between each of the additional concentric interventional devices in the concentric stack. In certain embodiments, the fluid lumen can include a lumen between a distal hub and a proximal interventional device, such as, for example, the lumen between the guide catheter hub 2914 and the procedure catheter 2904. In certain embodiments, priming can be performed while the devices are still in the sterile packaging.

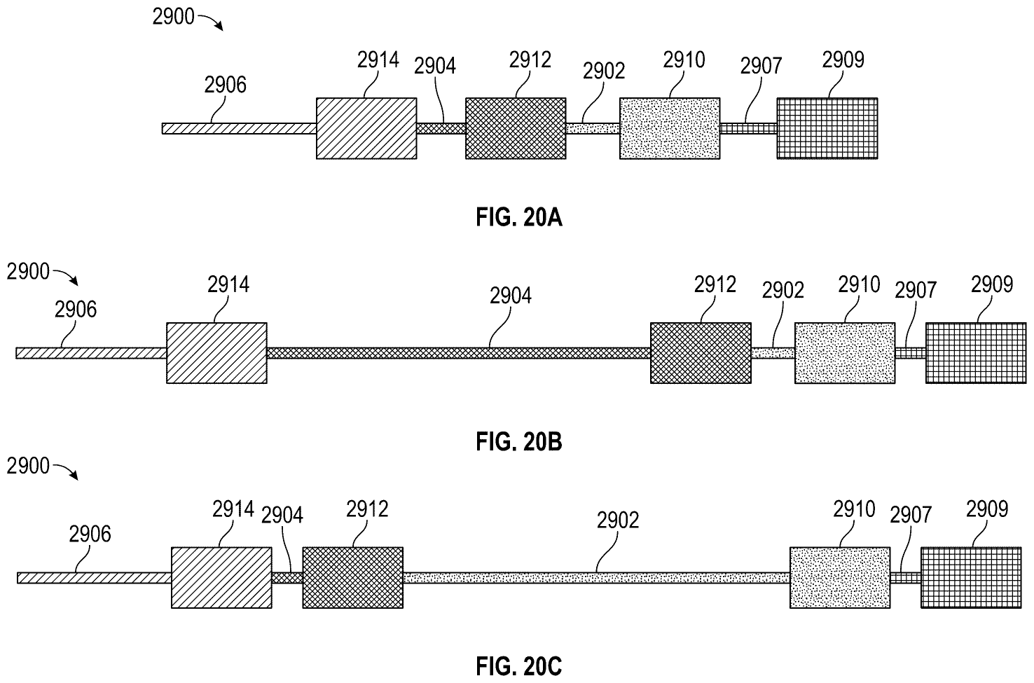

The fluidics connections can be connected to a fluidics system for delivering saline and contrast media to the catheters and providing aspiration. In some embodiments, the fluidics connections may be passed outside the sterile field for connection to the fluidics system. Once connected, the fluidics system can perform a priming sequence to flush each catheter of the interventional device assembly with fluid (e.g., saline, contrast media, or a mixture of saline and contrast media). The priming sequence may also include flushing each corresponding catheter hub with fluid. The fluid may be de-aired or de-gassed by the fluidics system prior to priming. In some embodiments, a vacuum source of the fluidics system can also be used to evacuate air from each catheter while flushing with fluid. In certain embodiments, a tip of the catheter can be placed into a container of fluid, such as saline, contrast media, or a mixture of saline and contrast media, during priming so that the fluid in the container, and not air, is aspirated through the tip of the catheter when the vacuum source is applied. In other embodiments, the tip of the catheter may be blocked (for example, using a plug) so that air is not aspirated from the tip of the catheter when the vacuum source is applied. In certain embodiments, the priming process may be automated such that a user can provide a single command and each catheter (and in some embodiments, each corresponding catheter hub) can be primed, sequentially (for example, as described with respect to FIGS. 20A-20C) or simultaneously.

Additional details regarding fluidics systems are disclosed in U.S. patent application Ser. No. 17/879,614, entitled Multi Catheter System With Integrated Fluidics Management, filed Aug. 2, 2022, and U.S. patent application Ser. No. 18/666,217, entitled Fluidics Control System For Multi Catheter Stack, filed May 16, 2024, each of which is hereby expressly incorporated by reference in its entirety herein.

Fluid resistance within a lumen may be greater when there is a reduction in cross sectional luminal area for flow, for example, when a second interventional device (e.g., a catheter or guidewire) extends within the lumen of a first interventional device. The amount of fluid resistance can be affected by the length of the cross sectional narrowing, for example, due to a depth of axial insertion of the second interventional device within the first interventional device. A second interventional device extending partially through the lumen of a first interventional device will provide a smaller length of cross-sectional narrowing, and accordingly may result in a lower fluid resistance within the lumen of the first catheter, than if the second interventional device were to extend entirely through the lumen of the first interventional device. Thus, fluid resistance can be lowered by at least partially decreasing a depth of axial insertion (i.e., axial overlap) of a second interventional device into the lumen through which fluid is to be injected (e.g., a length of the second interventional device into its concentrically adjacent lumen).

In some embodiments, over certain depths of insertion of a second interventional device within a first interventional device (for example, when the second interventional device is at or near a maximum insertion depth within the first interventional device), the size of the fluid channel between the devices (e.g., the annular lumen between the first interventional device and the second interventional device) can lead to higher than desirable amounts of fluid resistance during a priming procedure. In some embodiments, the depth of insertion of the second interventional device within the first interventional device can be decreased to reduce the pressure needed to prime the catheter and reduce internal interference.

In some embodiments, a catheter in the interventional device assembly can be separated from the other interventional devices for priming to reduce the pressure needed to prime the catheter and reduce internal interference. The catheter being primed may be separated from the interventional devices within the lumen of the catheter by proximally retracting the interventional devices within the lumen of the catheter. For example, the interventional devices within the lumen of the catheter being primed can be proximally retracted from the catheter being primed as far as possible while still maintaining a nested or stacked relationship (e.g., at least about 2 cm or 5 cm or more axial overlap) in order to minimize the pressure needed to prime the catheter and minimize internal interference. In other words, a catheter can be separated from more proximal interventional devices for priming while a distal tip of an adjacent proximal interventional device is still positioned within the lumen of the catheter. Maintaining at least some of the distal tip of an adjacent proximal interventional device within the lumen of the catheter may allow for easier reinsertion and advancement of the proximal interventional device after priming.

In some embodiments, the axial overlap may be between about 2 cm and about 20 cm, between about 2 cm and 10 cm, between about 2 cm and 5 cm, between about 5 cm and 20 cm, between about 5 cm and 10 cm, or any other suitable range. In some embodiments, the axial overlap may be at least about 2 cm, at least about 5 cm, at least about 10 cm, at least about 20 cm, no more than 2 cm, no more than 5 cm, no more than 10 cm, no more than 20 cm, about 2 cm, about 5 cm, about 10 cm, about 20 cm, or any other suitable amount.

In some embodiments, the robotic drive table can be programed to proximally retract the inner interventional device(s) from the catheter being primed as much as possible while still maintaining a nested or stacked relationship. In other embodiments, the robotic drive table can be programmed to separate inner devices from the catheter being primed to a distance sufficient to optimize the length of the unobstructed lumen and result in an amount of fluid resistance lower than a threshold value. After the catheter being primed is separated from the other interventional devices, the catheter can be primed by flushing the catheter with fluid, such as saline, contrast media, or a mixture of saline and contrast media.

After the catheter is primed, it may be returned to an initial position and a next catheter of the interventional device assembly can be separated from the other interventional devices within its lumen for priming. This sequence can be repeated for each catheter of the interventional device assembly. In other embodiments, after a catheter is primed, it may be advanced to a ready or drive position to begin insertion into the patient. While the foregoing describes separating catheters to be primed by retraction of inner interventional devices, an outer catheter may also be separated from inner interventional devices by distally axially advancing the outer catheter relative to the inner interventional devices. An example of a priming process is described with respect to FIGS. 20A-20C.

FIG. 20A depicts the interventional device assembly 2900 assembled in a concentric stack and axially compressed configuration. As shown in FIG. 20A, the interventional devices can be fully nested within each other. This may be the configuration following unpackaging of the device assembly 2900 and placement onto the robotic drive table. A priming sequence may begin by distally axially advancing the guide catheter 2906 and guide catheter hub 2914 relative to the procedure catheter 2904, procedure catheter hub 2912, catheter 2902, insert or access catheter hub 2910, guidewire 2907, and guidewire hub 2909, for example, as far as possible while maintaining a distal tip of the procedure catheter 2904 within the lumen of the guide catheter 2906, as shown in FIG. 20B, or to a distance that will result in a desirable amount of fluid resistance for priming. In some embodiments, the guide catheter 2906 is advanced in response to a control signal from a control system. The guide catheter 2906 can then be primed by introducing priming fluid using the fluidics system. In some embodiments, priming fluid is introduced in response to a control signal from a control system. Priming the guide catheter 2906 can include priming the guide catheter hub 2914. For example, in certain embodiments, the guide catheter hub 2914 or a hemostasis valve coupled thereto can include fluidics connections to receive priming fluid from the fluidics system. After priming, the guide catheter 2906 can be returned to its initial position (e.g., the fully axially compressed configuration) as shown in FIG. 20A. In some embodiments, the guide catheter 2906 is returned to its initial position in response to a control signal from a control system.

After the guide catheter 2906 is primed and returned to its initial position, the procedure catheter 2904 and procedure catheter hub 2912 can be distally axially advanced relative to the catheter 2902, insert or access catheter hub 2910, guidewire 2907 and guidewire hub 2909 (also distally axially advancing the guide catheter 2906 and guide catheter hub 2914 without changing or minimally changing their relative position with respect to procedure catheter 2904), for example, as far as possible while maintaining a distal tip of the catheter 2902 within the lumen of the procedure catheter 2904, as shown in FIG. 20C, or to a distance that will result in a desirable amount of fluid resistance for priming. In some embodiments, the procedure catheter 2904 and the guide catheter 2906 are advanced in response to a control signal from a control system. The procedure catheter 2904 can then be primed by introducing priming fluid using the fluidics system. In some embodiments, priming fluid is introduced in response to a control signal from a control system. Priming the procedure catheter 2904 can include priming the procedure catheter hub 2912. For example, in certain embodiments, the procedure catheter hub 2912 or a hemostasis valve coupled thereto can include fluidics connections to receive priming fluid from the fluidics system. After priming, the procedure catheter 2904 and guide catheter 2906 can be returned to their initial positions (e.g., the fully axially compressed configuration) as shown in FIG. 20A. In some embodiments, the procedure catheter 2904 and the guide catheter 2906 are returned to their initial position in response to a control signal from a control system.

After the procedure catheter 2904 is primed and returned to its initial position, the catheter 2902 and insert or access catheter hub 2910 can be distally axially advanced relative to the guidewire 2907 and guidewire hub 2909 (also distally axially advancing the guide catheter 2906, guide catheter hub 2914, procedure catheter 2904, and procedure catheter hub 2912 without changing or minimally changing their relative positions with respect to the catheter 2902), for example, as far as possible while maintaining a distal tip of the guidewire 2907 within the lumen of the catheter 2902, or to a distance that will result in a desirable amount of fluid resistance for priming. In some embodiments, the catheter 2902, the procedure catheter 2904, and the guide catheter 2906 are advanced in response to a control signal from a control system. The catheter 2902 can then be primed by introducing priming fluid using the fluidics system. In some embodiments, priming fluid is introduced in response to a control signal from a control system. Priming the catheter 2902 can include priming the insert or access catheter hub 2910. For example, in certain embodiments, the insert or access catheter hub 2910 or a hemostasis valve coupled thereto can include fluidics connections to receive priming fluid from the fluidics system. After priming, the catheter 2902 and catheters 2904 and 2906 can be returned to their initial positions (e.g., the fully axially compressed configuration) shown in FIG. 20A. In some embodiments, the catheter 2902, the procedure catheter 2904, and the guide catheter 2906 are returned to their initial position in response to a control signal from a control system.

In some embodiments, the priming procedure described with respect to FIGS. 20A-20C may be performed in response to a single control signal from a control system. In other embodiments, various steps of the priming procedure may be performed in response to unique control signals. In some embodiments, priming of each unique interventional device can be performed in response to a unique control signal.

In alternative embodiments, each of the catheters can be distally separated from one another simultaneously for priming. For example, the catheter 2902 can be distally separated from the guidewire 2907 while maintaining the distal tip of the guidewire 2907 in the lumen of the catheter 2902, the procedure catheter 2904 can be distally separated from the catheter 2902 while maintaining the distal tip of the catheter 2902 in the lumen of the procedure catheter 2904, and the guide catheter 2906 can be distally separated from the procedure catheter 2904 while maintaining the distal tip of the procedure catheter 2904 in the lumen of the guide catheter 2906 simultaneously. However, an embodiment in which only one set of adjacent hubs is separated at a time, as described with respect to FIGS. 20A-20C, can provide a smaller overall length of the assembly at any particular time, which can allow for use with a smaller robotic drive system. While separation of outer catheters from their inner interventional devices is described as distally axially advancing the catheters relative to their inner interventional devices, separation can include proximally retracting the inner interventional devices from the outer catheters.

In alternative embodiments, one or more of the catheter 2902, the procedure catheter 2904, and the guide catheter 2906 can be advanced to a ready or drive position to begin insertion into the patient after priming (e.g., prior to priming a subsequent catheter). In such embodiments, the catheters may advance to the ready or drive position without returning to their initial position after priming.

As described above, in some embodiments, the catheters 2902, 2904, and 2906 may be assembled into the concentric stack orientation illustrated in FIG. 17 prior to flushing the catheters to remove air by displacing it with a fluid such as saline contrast media, or a mixture of saline and contrast media. This is preferably accomplished in each fluid lumen, such as, for example, the annular lumen between the guide catheter 2906 and the procedure catheter 2904 and in between each of the additional concentric interventional devices in the concentric stack. Infusing fluid (e.g., saline, contrast media, or a mixture of saline and contrast media) under pressure may displace substantially all of the air but some small bubbles may remain, adhering to the inside wall of an outer catheter (e.g., the guide catheter 2906), the outside wall of an inner catheter (e.g., the procedure catheter 2904), or both.

While fluid is being introduced under pressure into the proximal end of the annular lumen (e.g., into a hub of the outer catheter or a hemostasis valve coupled thereto), the inner catheter may be moved with respect to the outer catheter, to disrupt the holding forces between the microbubbles and adjacent wall and allow the bubbles to be carried downstream and out through the distal opening of the lumen or removed via aspiration. The catheters may be moved axially, rotationally or both with respect to each other. In certain embodiments, the catheters may be reciprocated axially, rotationally, or both with respect to each other. In some embodiments, the catheters may be moved intermittently axially, rotationally, or both. In other embodiments, the catheters may be rotated continuously or in a constant direction.

In some implementations, a first catheter is moved reciprocally with respect to an adjacent catheter or guidewire such as axially over a stroke length in a range of from about 1 mm to about 250 mm, from about 10 mm to about 250 mm, from about 5 mm to about 125 mm, from about 25 mm to about 125 mm, from about 10 mm to about 50 mm, from about 15 mm to about 30 mm, from about 5 mm to about 30 mm, from about 15 mm to about 25 mm, from about 20 mm to about 40 mm, or any other suitable range. In some implementations, a first catheter is moved reciprocally with respect to an adjacent catheter or guidewire such as axially over a stroke length of at least 5 mm, at least 10 mm, at least 15 mm, at least 20 mm, at least 25 mm, at least 30 mm, at least 50 mm, no more than 10 mm, no more than 20 mm, no more than 25 mm, no more than 30 mm, no more than 50 mm, no more than 125 mm, no more than 150 mm, about 5 mm, about 10 mm, about 15 mm, about 20 mm, about 25 mm, about 30 mm, about 50 mm, or any other suitable stroke length.

In some implementations, a first catheter is moved reciprocally with respect to an adjacent catheter or guidewire such as axially at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as axially at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In one implementation, a first catheter is moved reciprocally with respect to the adjacent catheter or guidewire such as axially over a stroke length in a range of from about 0.5 inches to about 10 inches, or from about one inch to about 5 inches at a reciprocation frequency of no more than about 5 cycles per second or two cycles per second or less.

In some implementations, a first catheter is moved reciprocally with respect to an adjacent catheter or guidewire such as rotationally over an angle of rotation per stroke in a range of from about 5 degrees to about 180 degrees, from about 5 degrees to about 360 degrees, from about 15 degrees to about 180 degrees, from about 15 degrees to about 150 degrees, from about 15 degrees to about 120 degrees, from about 15 degrees to about 90 degrees, form about 15 degrees to about 60 degrees, from about 15 degrees to about 30 degrees, from about 30 degrees to about 180 degrees, from about 30 degrees to about 150 degrees, from about 30 degrees to about 120 degrees, from about 30 degrees to about 90 degrees, form about 30 degrees to about 60 degrees, from about 60 degrees to about 180 degrees, from about 60 degrees to about 150 degrees, from about 60 degrees to about 120 degrees, from about 60 degrees to about 90 degrees, from about 90 degrees to about 180 degrees, from about 90 degrees to about 150 degrees, from about 90 degrees to about 120 degrees, from about 120 degrees to about 180 degrees, from about 120 degrees to about 150 degrees, from about 150 degrees to about 180 degrees or any other suitable range. In some implementations, a first catheter is moved reciprocally with respect to an adjacent catheter or guidewire such as rotationally over an angle of rotation per stroke of at least 5 degrees, at least 15 degrees, at least 30 degrees, at least 60 degrees, at least 90 degrees, at least 120 degrees, at least 150 degrees, at least 180 degrees, at least 360 degrees, no more than 5 degrees, no more than 15 degrees, no more than 30 degrees, no more than 60 degrees, no more than 90 degrees, no more than 120 degrees, no more than 150 degrees, no more than 180 degrees, no more than 360 degrees, about 5 degrees, about 15 degrees, about 30 degrees, about 60 degrees, about 90 degrees, about 120 degrees, about 150 degrees, about 180 degrees, about 360 degrees, or any other suitable angle.

In some implementations, a first catheter is moved reciprocally with respect to an adjacent catheter or guidewire such as rotationally at a reciprocation frequency in a range of from about 0.5 Hz to about 1 Hz, from about 1 Hz to about 5 Hz, from about 1 Hz to about 10 Hz, from about 1 Hz to about 25 Hz, from about 5 Hz to about 10 Hz, from about 10 Hz to about 25 Hz, or any other suitable range of frequencies. In some implementations, the first catheter is moved reciprocally with respect to an adjacent catheter or guidewire such as rotationally at a reciprocation frequency of at least 0.5 Hz, at least 1 Hz, at least 2 Hz, at least 5 Hz, at least 10 Hz, at least 25 Hz, no more than 0.5 Hz, no more than 1 Hz, no more than 2 Hz, no more than 5 Hz, no more than 10 Hz, no more than 25 Hz, about 0.5 Hz, about 1 Hz, about 2 Hz, about 5 Hz, about 10 Hz, about 25 Hz or any other suitable frequency.

In some implementations, a first catheter is moved reciprocally with respect to an adjacent catheter or guidewire for a number of reciprocations between 1 and 200, between 1 and 100, between 1 and 50, between 1 and 25, between 1 and 15, between 1 and 10, between 1 and 5, between 5 and 25, between 5 and 15, between 5 and 10, or any other suitable range. In some implementations, a first catheter is moved reciprocally with respect to an adjacent catheter or guidewire for at least 1 reciprocation, at least 2 reciprocations, at least 5 reciprocations, at least 10 reciprocations, at least 15 reciprocations, at least 25 reciprocations, at least 50 reciprocations, no more than 5 reciprocations, no more than 10 reciprocations, no more than 15 reciprocations, no more than 25 reciprocations, no more 50 than reciprocations, no more than 100 reciprocations, no more than 200 reciprocations, about 1 reciprocation, about 2 reciprocations, about 5 reciprocations, about 10 reciprocations, about 25 reciprocations, about 50 reciprocations, about 100 reciprocations, about 200 reciprocations, or any other suitable number. One reciprocation can include a movement (axially or rotationally) from a first position to a second position followed by a return from the second position to the first position.

In some implementations, a first catheter is moved reciprocally with respect to an adjacent catheter or guidewire over a length of time in a range of from 1 about second to about 60 seconds, from about 1 second to about 45 seconds, from about 1 second to about 30 seconds, from about 1 second to about 20 seconds, from about 1 second to about 15 seconds, from about 1 second to about 10 seconds, from about 5 seconds to about 45 seconds, from about 5 seconds to about 30 seconds, from about 5 seconds to about 20 seconds, from about 5 seconds to about 15 seconds, from about 5 seconds to about 10 seconds, from about 10 seconds to about 30 seconds, form about 10 seconds to about 20 seconds, or any other suitable range. In some implementations, a first catheter is moved reciprocally with respect to an adjacent catheter or guidewire over a length of time of at least 1 second, at least 5 seconds, at least 10 seconds, at least 15 seconds, at least 20 seconds, at least 30 seconds, at least 45 seconds, at least 60 seconds, no more than 5 seconds, no more than 10 seconds, no more than 15 seconds, no more than 20 seconds, no more than 30 seconds, no more than 45 seconds, no more than 60 seconds, about 5 seconds, about 10 seconds, about 15 seconds, about 20 seconds, about 30 seconds, about 45 seconds, about 60 seconds, or any other suitable length of time.

Reciprocation of adjacent catheters to disrupt microbubbles may be accomplished manually by grasping the corresponding catheter hubs and manually moving the catheters axially or rotationally with respect to each other while delivering pressurized fluid (e.g., saline, contrast media, or a mixture of saline and contrast media). Alternatively, such as in a robotically driven system, a processor may be configured to robotically drive at least one of two adjacent catheter hubs (for example, at least one of guide catheter hub 2914 and procedure catheter hub 2912) to achieve relative movement between the adjacent catheters thereby disrupting and expelling microbubbles, such as in response to user activation of a flush control. For example, in certain embodiments, two adjacent interventional devices may be moved relative to one another in response to a control signal from a control system. In certain embodiments, delivery of pressurized fluid may be performed in response to a control signal from a control system.

The reciprocation of adjacent catheters may generate shear forces that dislodge the air bubbles. For example, relative movement of the inner and outer surfaces of adjacent catheters may increase the fluid shear rate between the adjacent catheters during priming in comparison to static surfaces. In some embodiments, the shear force can be increased by increasing the flow rate of the solution (e.g., saline, contrast media, or a mixture of saline and contrast media) being provided by the fluidics system. In certain embodiments, both flow rate and relative movement between adjacent catheters are controlled to dislodge air bubbles.

In some embodiments, after each catheter is primed by the fluidics system, an ultrasound bubble detector may be used to confirm that the catheters are substantially free of air bubbles. For example, an ultrasound chip (such as mounted within a hub adjacent a catheter receiving lumen) may be run along the length of the catheters to confirm that no air bubbles remain in the system.

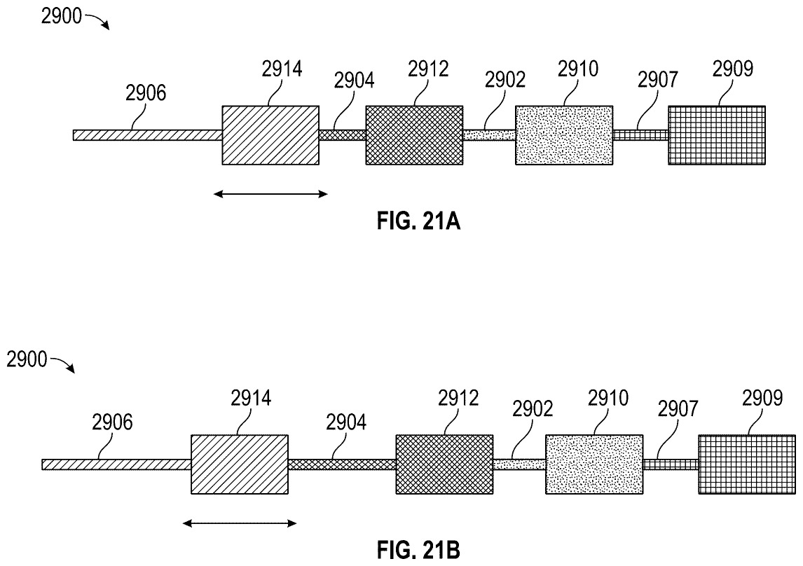

An example of a priming process including reciprocal movement of adjacent catheters is described with respect to FIGS. 21A-21B.

FIG. 21A depicts the interventional device assembly 2900 assembled in a concentric stack configuration. As shown in FIG. 21A, the interventional devices can be fully nested within each other. This may be the configuration following unpackaging of the device assembly 2900 and placement onto the robotic drive table. Alternatively, individual interventional devices of the device assembly 2900 can be assembled into the device assembly 2900 on the drive table.

A priming sequence may begin by priming the guide catheter 2906. In some embodiments, the guide catheter 2906 can be primed by introducing fluid (e.g., saline, contrast media, or a mixture of saline and contrast media) under pressure into the lumen of the guide catheter 2906 while generating reciprocal movement of guide catheter 2906 and/or guide catheter hub 2914, axially, rotationally or both, relative to the procedure catheter 2904. Priming the guide catheter 2906 can include priming the guide catheter hub 2914. For example, in certain embodiments, the guide catheter hub 2914 or a hemostasis valve coupled thereto can include fluidics connections to receive priming fluid from the fluidics system. In certain embodiments, the guide catheter 2906 and/or guide catheter hub 2914 can be axially agitated back and forth along a longitudinal axis of the guide catheter 2906 (e.g., between the position of FIG. 21A and the position of FIG. 21B). Axial and/or rotational reciprocal motion of the guide catheter 2906 and/or guide catheter hub 2914 can be performed manually or by a robotic drive table. Reciprocal movement may be generated in response to a control signal from a control system. Introducing fluid under pressure may be performed in response to a control signal from a control system.

In some embodiments, priming of the guide catheter 2906 may be performed by introducing fluid (e.g., saline, contrast media, or a mixture of saline and contrast media) under pressure into the lumen of the guide catheter 2906 while generating reciprocal movement of the procedure catheter 2904 and/or procedure catheter hub 2912, axially, rotationally or both, relative to the guide catheter 2906. Axial and/or rotational reciprocal motion of the procedure catheter 2904 and/or procedure catheter hub 2912 can be performed manually or by a robotic drive table. Reciprocal movement may be generated in response to a control signal from a control system. Introducing fluid under pressure may be performed in response to a control signal from a control system.

In some embodiments, priming of the guide catheter 2906 may be performed by introducing fluid (e.g., saline, contrast media, or a mixture of saline and contrast media) under pressure into the lumen of the guide catheter 2906 while generating reciprocal movement of both the guide catheter 2906 (and/or guide catheter hub 2914) and the procedure catheter 2904 (and/or procedure catheter hub 2912), axially, rotationally or both, relative to one another. Reciprocal movement may be generated in response to a control signal from a control system. Introducing fluid under pressure may be performed in response to a control signal from a control system.

In some embodiments, after priming the guide catheter 2906, the guide catheter 2906 can be returned to an initial position as shown in FIG. 21A. In other embodiments, after priming the guide catheter 2906, the guide catheter 2906 can be advanced to a ready or drive position to begin insertion into the patient.

In some embodiments, after the guide catheter 2906 is primed, the procedure catheter 2904 can be primed. Priming the procedure catheter 2904 can include priming the procedure catheter hub 2912. For example, in certain embodiments, the procedure catheter hub 2912 or a hemostasis valve coupled thereto can include fluidics connections to receive priming fluid from the fluidics system. In some embodiments, the procedure catheter 2904 can be primed by introducing fluid (e.g., saline, contrast media, or a mixture of saline and contrast media) under pressure into the lumen of the procedure catheter 2904 while generating reciprocal movement of the procedure catheter 2904 and/or procedure catheter hub 2912, axially, rotationally or both, relative to the catheter 2902. Reciprocal movement may be generated in response to a control signal from a control system. Introducing fluid under pressure may be performed in response to a control signal from a control system.

In some embodiments, priming of the procedure catheter 2904 may be performed by introducing fluid (e.g., saline, contrast media, or a mixture of saline and contrast media) under pressure into the lumen of the procedure catheter 2904 while generating reciprocal movement of the catheter 2902 and/or insert or access catheter hub 2910, axially, rotationally or both, relative to the procedure catheter 2904. Axial and/or rotational reciprocal motion of the catheter 2902 and/or insert or access catheter hub 2910 can be performed manually or by a robotic drive table. Reciprocal movement may be generated in response to a control signal from a control system. Introducing fluid under pressure may be performed in response to a control signal from a control system.

In some embodiments, priming of the procedure catheter 2904 may be performed by introducing fluid (e.g., saline, contrast media, or a mixture of saline and contrast media) under pressure into the lumen of the procedure catheter 2904 while generating reciprocal movement of both the procedure catheter 2904 (and/or procedure catheter hub 2912) and the catheter 2902 (and/or insert or access catheter hub 2910), axially, rotationally or both, relative to one another. Reciprocal movement may be generated in response to a control signal from a control system. Introducing fluid under pressure may be performed in response to a control signal from a control system.

In some embodiments, after priming the procedure catheter 2904, the procedure catheter 2904 can be returned to an initial position as shown in FIG. 21A. In some embodiments, after priming the procedure catheter 2904, the procedure catheter 2904 can be advanced to a ready or drive position to begin insertion into the patient.

In some embodiments, after the procedure catheter 2904 is primed, the catheter 2902 can be primed. Priming the catheter 2902 can include priming the insert or access catheter hub 2910. For example, in certain embodiments, the insert or access catheter hub 2910 or a hemostasis valve coupled thereto can include fluidics connections to receive priming fluid from the fluidics system. In some embodiments, the catheter 2902 can be primed by introducing fluid (e.g., saline, contrast media, or a mixture of saline and contrast media) under pressure into the lumen of the catheter 2902 while generating reciprocal movement of the catheter 2902 and/or insert or access catheter hub 2910, axially, rotationally or both, relative to the guidewire 2907. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of the guidewire 2907 and/or guidewire hub 2909, axially, rotationally or both, relative to the catheter 2902. Axial and/or rotational reciprocal motion of the guidewire 2907 and/or guidewire hub 2909 can be performed manually or by a robotic drive table. Reciprocal movement may be generated in response to a control signal from a control system. Introducing fluid under pressure may be performed in response to a control signal from a control system.

In some embodiments, priming of the catheter 2902 may be performed by introducing fluid (e.g., saline, contrast media, or a mixture of saline and contrast media) under pressure into the lumen of the catheter 2902 while generating reciprocal movement of both the catheter 2902 (and/or insert or access catheter hub 2910) and the guidewire 2907 (and/or guidewire hub 2909), axially, rotationally or both, relative to one another. Reciprocal movement may be generated in response to a control signal from a control system. Introducing fluid under pressure may be performed in response to a control signal from a control system.

In some embodiments, after priming the catheter 2902, the catheter 2902 can be returned to an initial position as shown in FIG. 21A. In other embodiments, after priming the catheter 2902, the catheter 2902 can be advanced to a ready or drive position to begin insertion into the patient.

In some embodiments, the priming procedure described with respect to FIGS. 21A and 21B may be performed in response to a single control signal from a control system. In other embodiments, various steps of the priming procedure may be performed in response to unique control signals. In some embodiments, priming of each unique interventional device can be performed in response to a unique control signal.

In the priming sequence described herein with respect to FIGS. 21A and 21B, the catheters are primed in order starting with the guide catheter 2906, followed by the procedure catheter 2904, and then followed by the catheter 2902. However, it is contemplated that the catheters may be primed in any order. The catheters may be primed in series as described above with respect to FIGS. 21A and 21B. Alternatively, two or more of the catheters or each of the catheters may be primed in parallel.

In certain embodiments, priming the catheters can include decreasing a depth of axial insertion (i.e., axial overlap) of a second interventional device into the lumen of a first interventional device through which fluid is to be injected (e.g., a length of the second interventional device into its concentrically adjacent lumen), as described with respect to FIGS. 20A-20C, and also generating relative reciprocal movement, axially, rotationally or both, between first interventional device and the second interventional device during priming, as discussed with respect to FIGS. 21A and 21B.

In some implementations, priming of a catheter can include vibrating at least a portion of the catheter and/or its associated hub when included. Vibration can be induced, for example, by an electric motor incorporated into a hub of the catheter, or by a separate electric motor or source of vibration put against the catheter when priming. In some implementations, at least a portion of the support table on which the catheters and/or their associated hubs are placed upon can vibrate during priming of any one or more catheters to aid in removal of air and/or microbubbles of air. Such vibration can be performed by an electric motor.

### EXAMPLES

Additional embodiments are disclosed m further detail in the following examples, which are not in any way intended to limit the scope of the claims.

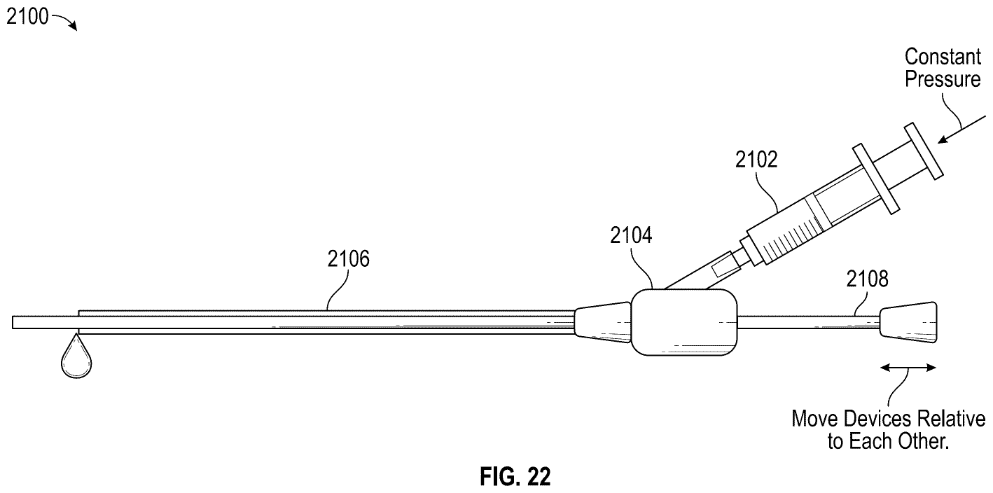

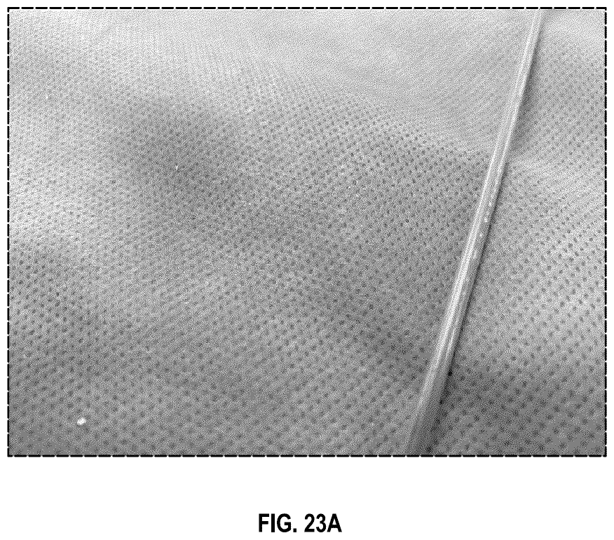

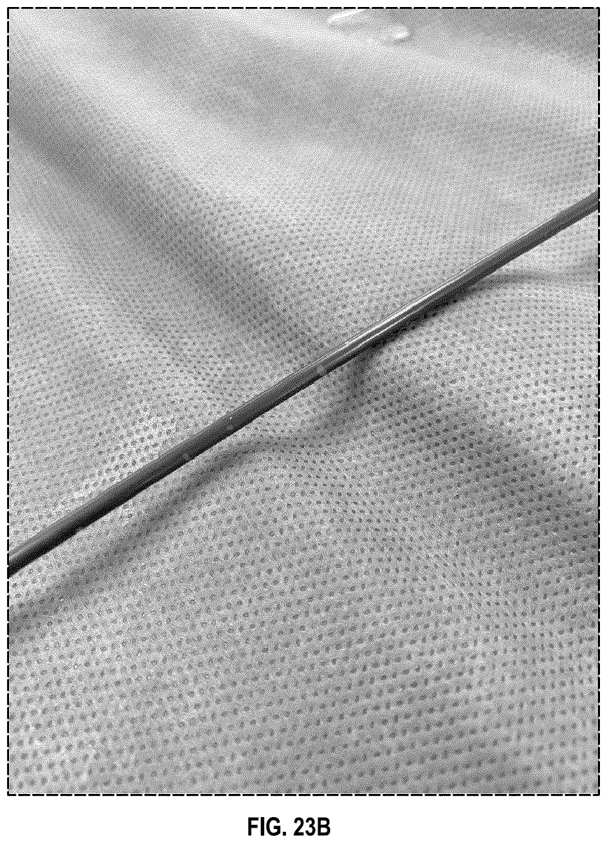

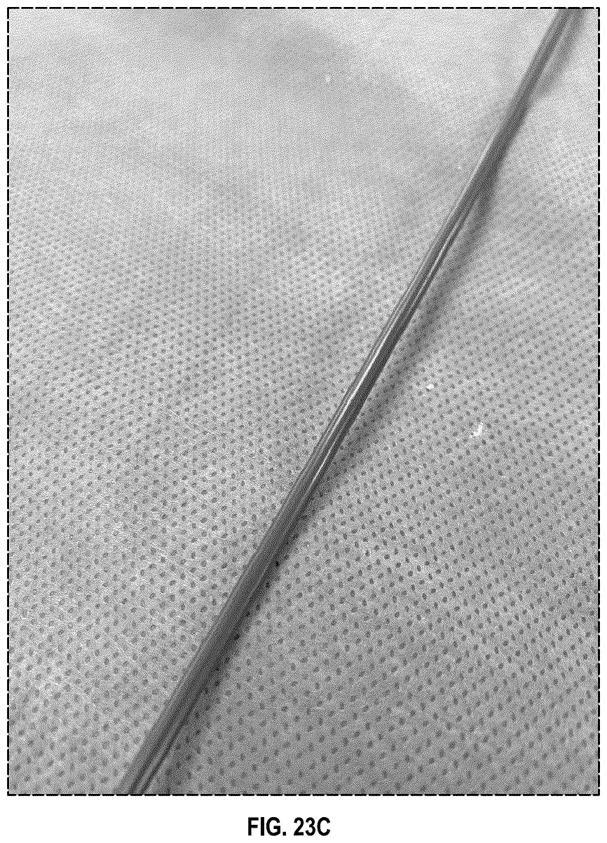

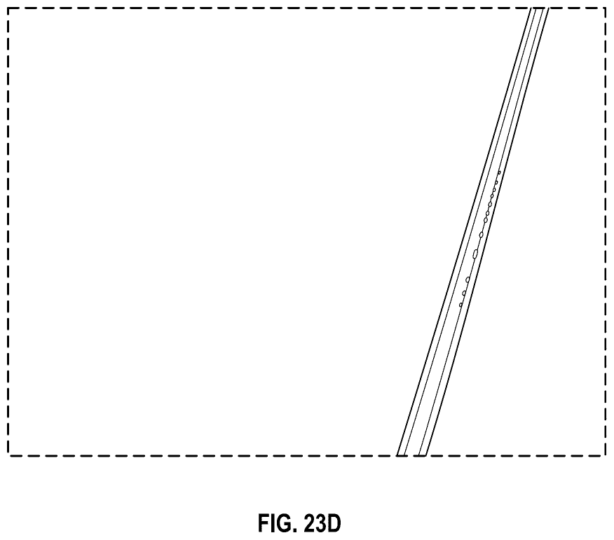

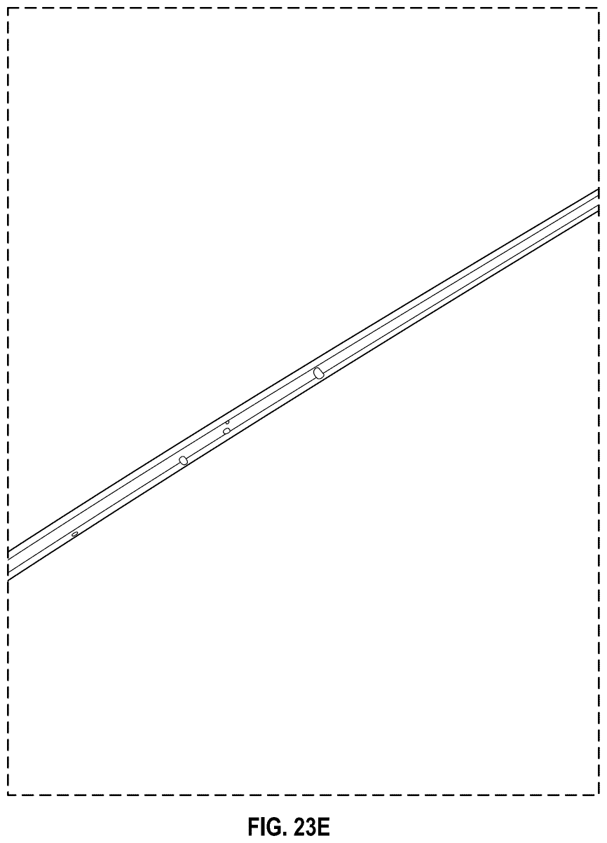

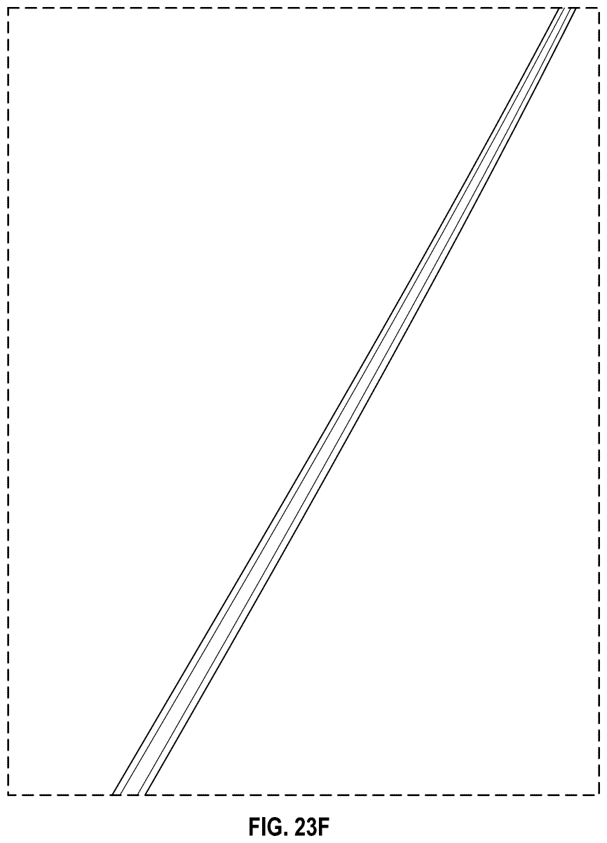

FIG. 22 is a diagram of a test system that was used for detecting the removal of air bubbles between concentrically stacked catheters. The test system included an inner catheter 2108 positioned within an interior lumen of an outer catheter 2106 in a concentric stack. The outer catheter 2106 was coupled to a rotating hemostasis valve 2104. The hemostasis valve 2104 was coupled to a syringe 2102 so that fluid injected using the syringe would flow through the lumen between the inner catheter 2108 and the outer catheter 2016. In the test system, the inner catheter 2108 had a diameter of about 0.071 inches. The outer catheter 2106 had a diameter of about 0.088 inches. The outer catheter 2106 was transparent to permit visualization of bubbles within the lumen. A distal end of the outer catheter 2108 allowed for small volumes of fluid to exit the outer catheter. FIG. 23A is a photograph showing the catheter 2106 and catheter 2108 in a concentric stack, prior to injection of fluid. FIG. 23D is an illustration thereof.

### Example 1

In a first example, the syringe 2102 was used to inject water at a constant pressure of about 150 psi through the hemostasis valve 2104 without moving the catheter 2106 or the catheter 2108. FIG. 23B is a photograph showing the catheter 2106 and catheter 2108 following the injection of water. FIG. 23E is an illustration thereof. As shown in FIG. 23B, bubbles are present within the lumen between the catheter 2106 and the catheter 2108.

### Example 2

In a second example, the syringe 2102 was used to inject water at a constant pressure of about 150 psi through the hemostasis valve 2104. Shortly after beginning to inject water, axial reciprocal movement of the inner catheter 2108 was performed for about 10 seconds. The reciprocal movement was performed at a frequency of about 1 Hz (or less) and a stroke length of about 20 mm (or more). FIG. 23C is a photograph showing the catheter 2106 and the catheter 2108 following the axial reciprocal movement. FIG. 23F is an illustration thereof. As shown in FIG. 23C, the lumen between the catheter 2106 and the catheter 2108 was substantially free of bubbles.

### Example 3

In a third example, an outer catheter having a diameter of about 0.071 inches and an inner catheter having a diameter of about 0.035 inches were used in the test system 2100 instead of the outer catheter 2106 and the inner catheter 2108 described with respect to Examples 1 and 2. A syringe 2102 was used to inject water at a constant pressure of about 150 psi through a hemostasis valve 2104 coupled to the outer catheter. Shortly after beginning to inject water, axial reciprocal movement of the inner catheter was performed for about 10 seconds. The reciprocal movement was performed at a frequency of about 1 Hz (or less) and a stroke length of about 20 mm (or more). Following the axial reciprocal movement, the lumen between the outer and inner catheters was found to be substantially free of bubbles by visual inspection.

### Control System

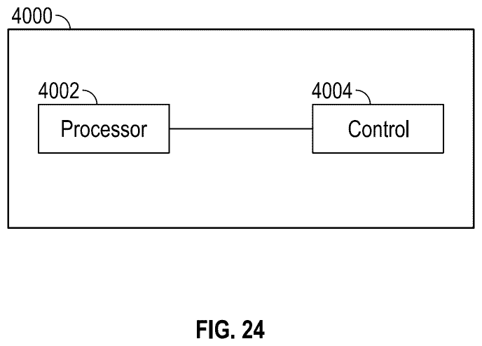

FIG. 24 illustrates a schematic view of an example of a control system 4000 that may be used to electronically control the systems and components described herein and/or perform the methods described herein. The control system 4000 may be configured to automatically adjust various motors, hub adapters, hubs, interventional devices, active torque elements, fluidics components (e.g., valves, pumps, etc.), and/or any other components described herein in response to commands input by an operator such as a physician. In response to command inputs by an operator, the control system 4000 may cause a series of responsive events to automatically occur.

In certain embodiments, the control system 4000 can include one or more processors 4002. The one or more processors 4002 can be configured to automatically adjust the various system components described herein in response to commands input by an operator, for example, using one or more controls 4004 of the control system 4000. A single control 4004 is shown in FIG. 24. However, any suitable number of controls may be provided to correspond to various functions of the systems described herein. For example, in certain embodiments, each interventional device may have its own unique control 4004 or set of controls 4004 that can control various functions of the interventional device (e.g., axial movement, rotational movement, supply of fluids (e.g., saline, contrast, etc.), aspiration, etc.).

In certain embodiments, one or more controls 4004 may control priming functions for one or more interventional devices. For example, one or more controls 4004 can be operated to cause the interventional devices to perform a priming procedure, as described for example, with reference to FIGS. 20A-C. For example, one or more controls 4004 can be operated to cause axial movement of one or more interventional devices relative to one or more other interventional devices (e.g., by causing axial movement of corresponding hubs and/or hub adapters). One or more controls 4004 can be operated to cause introduction of fluid into the lumen of an interventional device to prime the interventional device.

In certain embodiments, one or more controls 4004 may be operated to cause the interventional devices to perform a priming procedure, as described for example, with reference to FIGS. 21A-21B. For example, one or more controls 4004 can be operated to cause reciprocal movement (e.g., axial and/or rotational reciprocal movement) of one or more interventional devices relative to one or more other interventional devices (e.g., by causing reciprocal movement of corresponding hubs and/or hub adapters). One or more controls 4004 can be operated to cause introduction of fluid into the lumen of an interventional device to prime the interventional device (e.g., during relative reciprocal movement).

The processor 4002 may receive signals from the one or more controls 4004 and in response, initiate corresponding actions in the components of the systems described herein. For example, the processor 4002 may be configured to generate output signals that cause responsive actions to be performed by the components of the described herein.

The control system 4000 may receive signals from one or more sensors described herein. The processor 4002 may receive signals from one or more sensors described herein and in response, initiate corresponding actions in the components of the systems described herein.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

The foregoing represents one specific implementation of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

While the foregoing describes interventional devices that are driven by a drive table, other suitable robotic drive systems or mechanisms may be used to drive the interventional devices, as will be appreciated by those of skill in the art in view of the disclosure herein.

Various systems and methods are described herein primarily in the context of a neurovascular access or procedure (e.g., neurothrombectomy). However, the catheters, systems (e.g., drive systems), and methods disclosed herein can be readily adapted for any of a wide variety of other diagnostic and therapeutic applications throughout the body, including particularly intravascular procedures such as in the peripheral vasculature (e.g., deep venous thrombosis), central vasculature (pulmonary embolism), and coronary vasculature, as well as procedures in other hollow organs or tubular structures in the body.

In some embodiments, magnets (e.g., neodymium magnets) may be used to transmit torque through a sterile barrier. For example, a torque transfer system may transmit torque from outside of the sterile field (e.g., from a hub adapter) to within the sterile field (e.g., to a hub). The torque transfer system may also be referred to as a magnetic coupling or a rotational magnetic coupling. In certain embodiments, the hub adapter may be reusable (e.g., part of the capital equipment within an operating room) and the hub may be disposable. In some embodiments, a torque transfer system can facilitate the provision of torque to rotate an interventional device coupled to the hub without a motor and motor control board on the disposable hub, which may greatly reduce equipment and procedure costs. In addition, some embodiments of the torque transfer system disclosed herein eliminate the need for cable management to each hub, increasing simplicity and reducing the cost of the system. In some embodiments, a torque transfer system can facilitate the provision of torque to rotate other instruments of a robotic surgical system (e.g., a robotic surgical system for neurovascular procedures). For example, the torque transfer system can be used to actuate one or more valves, as described herein. Instruments that are used in a robotic surgical procedure or to prepare for a robotic surgical procedure, including interventional devices, may be referred to as surgical instruments herein.

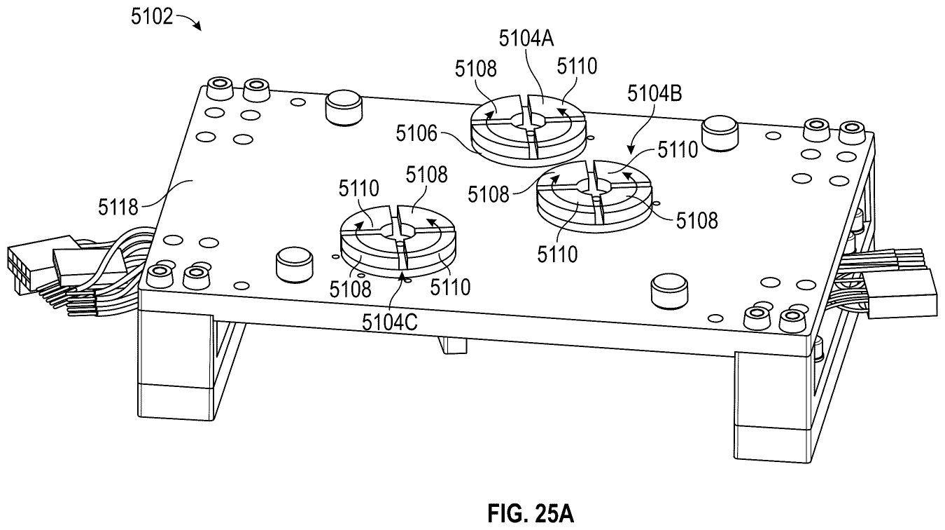

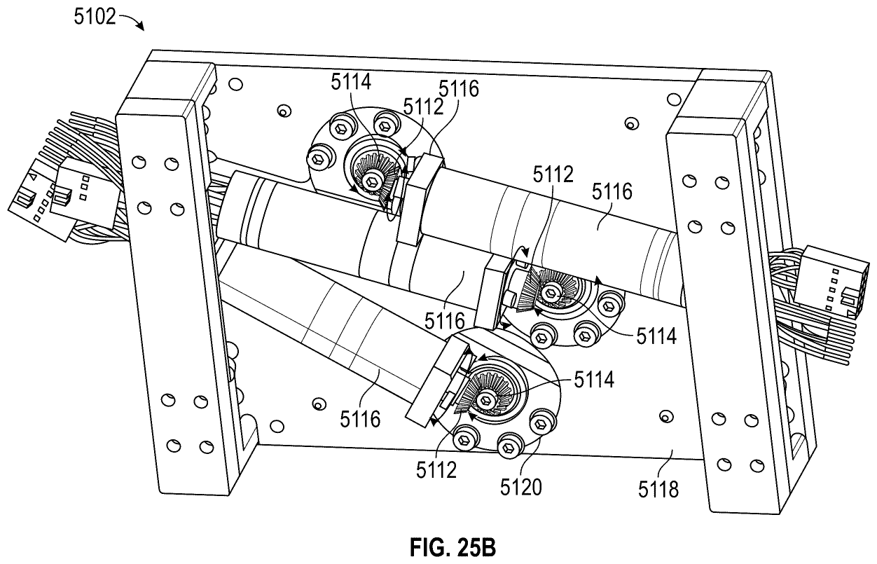

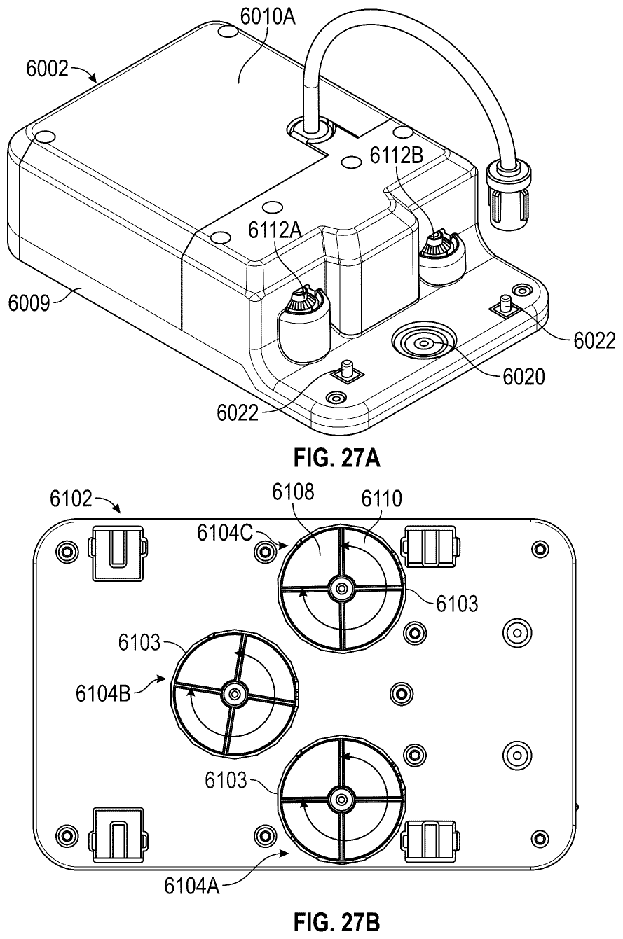

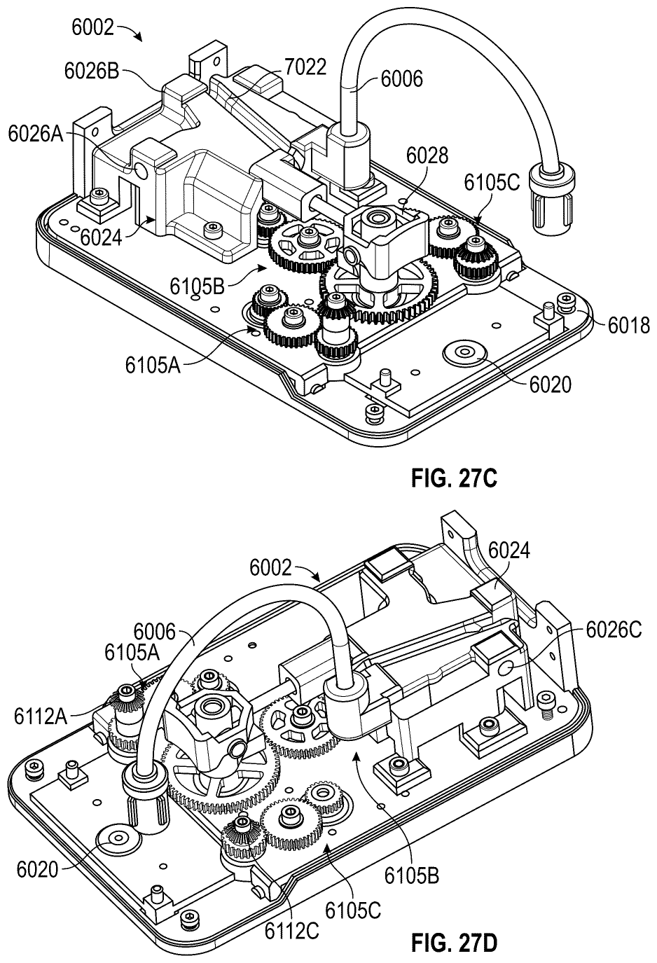

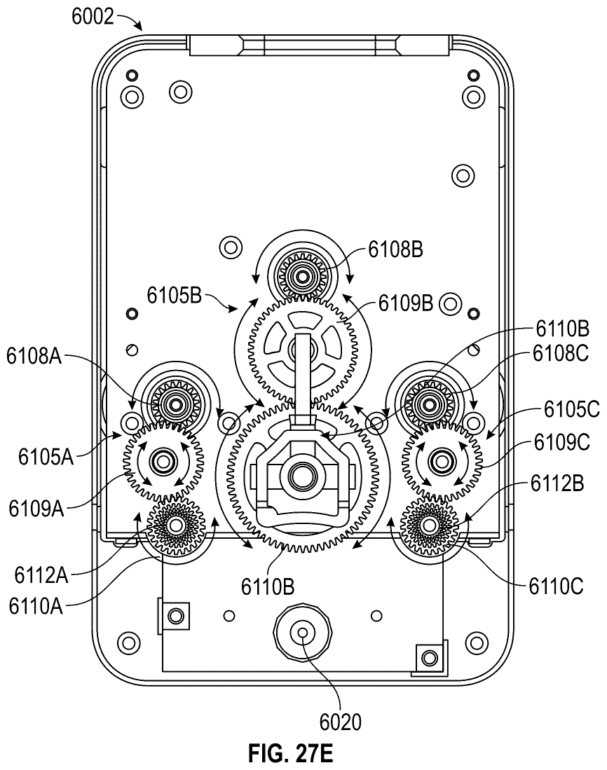

FIGS. 25A-25B depict an embodiment of an active torque subsystem 5102 of a torque transfer system that can be used to transfer a torque force through a sterile barrier. FIG. 27B depicts an embodiment of a passive torque subsystem 6102 of the torque transfer system, described in greater detail below. The active torque subsystem 5102 can be configured to be positioned on a nonsterile side (e.g., a capital equipment side) of a sterile barrier. The passive torque subsystem 6102 of the torque transfer system can be positioned on a sterile side (e.g., a disposable equipment side) of a sterile barrier.

The active torque subsystem 5102 can include one or more active torque elements 5104A, 5104B, 5104C. Each active torque element 5104A-5104C can include a plurality of magnets. The plurality of magnets can be arranged around a central axis. The plurality of magnets can include one or more first magnets 5108 and one or more second magnets 5110. The one or more first magnets 5108 can include a north or positive pole facing in a direction (e.g., axially towards the passive torque subsystem 6102). The one or more second magnets 5110 can include a south or negative pole facing in the same direction (e.g., axially towards the passive torque subsystem 6102). The magnets 5108 and 5110 can be wedge shaped magnets. In some embodiments, wedge shaped magnets may provide a greater magnetic surface area on a magnet support element in comparison to disc shaped magnets positioned on the same magnet support element.

In some embodiments, the plurality of magnets of the active torque element 5104A-5104C can be arranged in a planar arrangement on a plane parallel with or generally parallel with the sterile barrier.

As shown in FIG. 27B, the passive torque subsystem can include one or more passive torque elements 6104A, 6104B, and 6104C. Each passive torque element 6104A-C can include a plurality of magnets. The plurality of magnets can be arranged around a central axis. The plurality of magnets can include one or more first magnets 6108 and one or more second magnets 6110. The one or more first magnets 6108 can include a south or negative pole facing axially towards the active torque element. The one or more second magnets 6110 can include a north or positive pole facing axially towards the active torque element. The magnets 6108 and 6110 can be wedge shaped magnets. In some embodiments, the plurality of magnets of the passive torque element can be arranged in a planar arrangement on a plane parallel with or generally parallel with the sterile barrier.

The first magnets 5108 of the active torque element 5104A-C can be magnetically coupled with the first magnets 6108 of the passive torque element 6104A-C, and the second magnets 5110 of the active torque element 5104A-C can be coupled with the second magnets 6110 of the passive torque element 6104A-C. Rotation of the plurality of magnets of the active torque element 5104A-C about the central axis of the plurality of magnets of the active torque element 5104A-C can cause a corresponding rotation of the plurality of magnets of the passive torque element 6104A-C about the central axis of the plurality of magnets of the passive torque element 6104A-C.

In some embodiments, the central axis of the plurality of magnets of the active torque element 5104A-C can be coaxial with or parallel with the central axis of the plurality of magnets of the passive torque element 6104A-C. In some embodiments, the central axis of the plurality of magnets of the active torque element 5104A-C and/or the central axis of the plurality of magnets of the passive torque element 6104A-C can be transverse to (e.g., perpendicular to) a direction of axial movement of a hub adapter, hub, and/or interventional device to which components of torque transfer system is coupled.

With reference to FIGS. 25A-25B, each active torque element 5104A-C can include a magnet support element 5106 and the plurality of magnets 5108 and 5110. In some embodiments, the plurality of magnets 5108 and 5110 may be configured in an alternating pattern. For example, each magnet 5108 may be positioned adjacent to two magnets 5110 and each magnet 5110 may be positioned adjacent to two magnets 5108.

In certain embodiments, each of the one or more active torque elements 5104A-5104C can include a gear train, which can include a first gear 5112 coupled to a second gear 5114. In some embodiments, the first gear 5112 and the second gear 5114 can be miter gears (also referred to as bevel gears). The first gear 5112 can be coupled with a motor 5116. The motor 5116 can be configured to selectively exert a torque on the first gear 5112 to cause the first gear 5112 to rotate. The first gear 5112 can be configured to rotate the second gear 5114. The second gear 5114 can be coupled with the plurality of magnets 5108 and 5110 of the active torque element 5104. The plurality of magnets of the active torque element 5104 can be configured to rotate when the second gear 5112 rotates. In some embodiments, the second gear 5112 can be coupled to and configured to rotate the magnet support element 5106.

In some embodiments, the motor 5116 can be a servo motor. In some embodiments, as described herein, the torque transfer system can include a controller in electrical communication with the motor 5116 of the active torque element 5104 configured to control an operation of the motor 5116 in response to an input to the controller. The controller may be a microcontroller. In some embodiments, the motor 5116 can be coupled to a control system, such as control system 4000.

In certain embodiments, the active torque subsystem 5102 can include a housing or support plate 5118. The housing or support plate 5118 can include a plurality of recesses 5120 each configured to receive the magnet support elements 5106 of the active torque elements 5104A-5104C therein. The plurality of recesses 5120 can each be sized slightly larger than the magnet support elements 5106 so that the magnet support elements 7106 can rotate freely within the recesses 5120.

With reference to FIG. 27B, the passive torque subsystem 6102 may include a first passive torque element 6104A, a second passive torque element 6104B, and a third passive torque element 6104C. Each of the one or more passive torque elements 6104A-6104C can be configured to cause a responsive movement of a surgical instrument in response to a transfer of a magnetic force (e.g., a torque force) from an active torque element 5104 to a corresponding passive torque element. In some embodiments, each of the one or more passive torque elements 6104A-6104C may further include one or more magnets 6108, 6110. In some embodiments, the magnets 6108, 6110 may have opposing polarity. The passive torque subsystem 6102 can be configured to be positioned on a sterile side (e.g., a disposable equipment side) of a sterile barrier.

In certain embodiments, the one or more passive torque elements 6104A-6104C can be configured to control functions of a hub or hub assembly (e.g., hub assembly 6000). In some embodiments, the one or more passive torque elements 6104A-6104C can be configured to control medical devices or components of a robotic medical system. For example, in some embodiments, the one or more passive torque elements 6104A-6104D can be configured to control movement of an interventional device, and/or control fluidics components coupled to an interventional device (e.g., controllably select a fluidics configuration for a fluidics management system). For example, the one or more passive torque elements 6104A-6104C can be configured to rotate an interventional device, such as a catheter or guidewire, translate an interventional device, manipulate an interventional device (in the case of a deflectable tip catheter), and/or rotate a valve to controllably select an active fluidics configuration. In certain embodiments, the one or more passive torque elements 6104A-6104C may be configured to manipulate a sensor, for example, to position a sensor, such as a bubble sensor, in a desired position for operation. In certain embodiments, the one or more passive torque elements 6104A-6104C may be configured to increase tension in the system. For example, the one or more passive torque elements may cause the application of tension to a stent retriever to allow the stent retriever to pull other interventional devices.

In certain embodiments, the passive torque subsystem 6102 can include a housing or support plate. The housing can include a plurality of recesses each configured to receive the magnet support elements 6103 of the passive torque elements therein. The plurality of recesses can each be sized slightly larger than the magnet support elements 6103 so that the magnet support elements 6103 can rotate freely within the recesses.

In certain embodiments, one or more of the active torque elements 5104 can transmit up to 1.5 in-lb (0.17 N-m) of torque across a 0.2 inch air gap. In certain embodiments in which there are three active torque element and passive torque element pairs, the passive torque elements can collectively resist shear loads of up to 5.85 lb (26 N) across a 0.2 inch air gap. Shear refers to sliding of the magnets relative to each other in a horizontal plane. In some embodiments, the plurality of magnets of a single passive torque element may resist shear loads of 1.95 lb (8.7 N) across a 0.2 inch air gap. In some embodiments, each active torque element passive torque element pair may rotated at 1 revolution/sec with an acceleration of 1 revolution/sec². In some embodiments, the hub adapter may be configured to continuously resist 34 lbf pull force from the magnets across a 0.2 inch air gap.

In some embodiments, the magnetic coupling between one or more of the active torque element and passive torque element pairs described herein can be used to provide axial movement to an interventional device hub or hub assembly (for example, in addition to or alternatively to the drive magnet 67 and the driven magnet 69 discussed with respect to FIG. 4). For example, a plurality of magnets (e.g., magnets 5108 and 5110) of an active torque element 5104A-5104C of a hub adapter can act as a drive magnet arrangement configured to couple with a driven magnet arrangement formed by a plurality of magnets (e.g., magnets 6108 and 6110) of a passive torque element 6104A-6104C. This provides a magnetic coupling through the sterile barrier such that the hub or hub assembly (e.g., hub assembly 6000) is moved axially across the top of the sterile barrier in response to movement of the hub adapter axially outside of the sterile field.

For example, in certain embodiments, active torque elements 5104A-5104C of a hub adapter may be magnetically coupled with one or more of passive torque elements 6104A-6104C of a hub or hub assembly. In some embodiments, the hub adapter can be driven axially to cause a corresponding axial movement of the hub or hub assembly via the magnetic coupling between the active torque elements 5104A-5104C and the passive torque elements 6104A-6104C.

The magnetic force between magnets of a hub on the sterile side of a sterile barrier and magnets of a hub adapter on the non-sterile side of the barrier may counteract efforts to remove the hub from the drive table. In certain embodiments, one or more active torque element and passive torque element pairs may be actuated to create a repulsion force between the magnets of the active torque element and the passive torque element so as to reduce an overall magnetic attraction between the hub and the hub adapter, and consequently, the force needed to remove a hub from the drive table. In certain embodiments, a repulsion force may be generated by introducing a rotational misalignment between the magnets of the active torque element and magnets of the passive torque element.

As described herein, in certain embodiments, the active torque subsystem 5102 can be configured to be positioned on a nonsterile side (e.g., a capital equipment side) of a sterile barrier and the passive torque subsystem 6102 can be configured to be positioned on a sterile side (e.g., a disposable equipment side) of the sterile barrier. Further, in any embodiments disclosed herein, the torque transfer system can be configured for use in a surgical robotic drive system as described herein. For example, the active torque subsystem 5102 may be coupled to a hub adapter and the passive torque subsystem 6102 can be coupled to a hub or hub assembly of a robotic drive system.

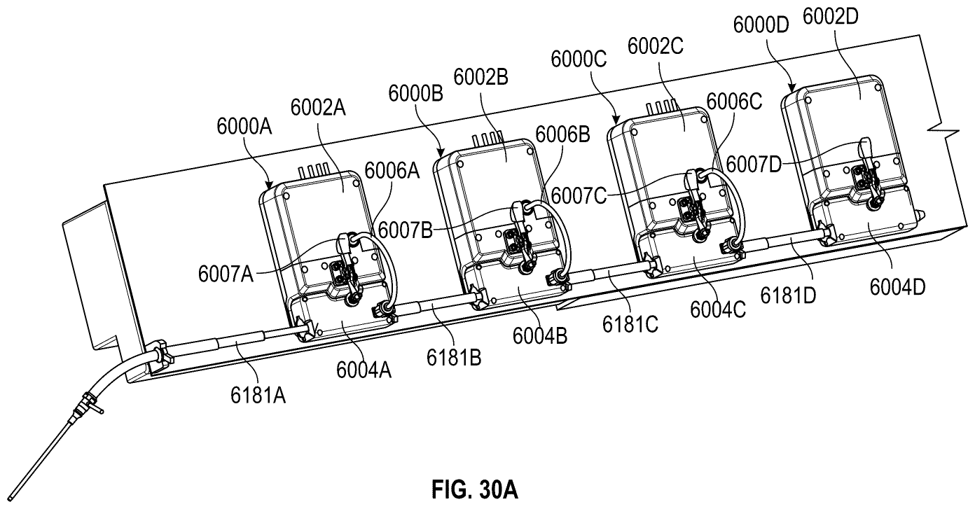

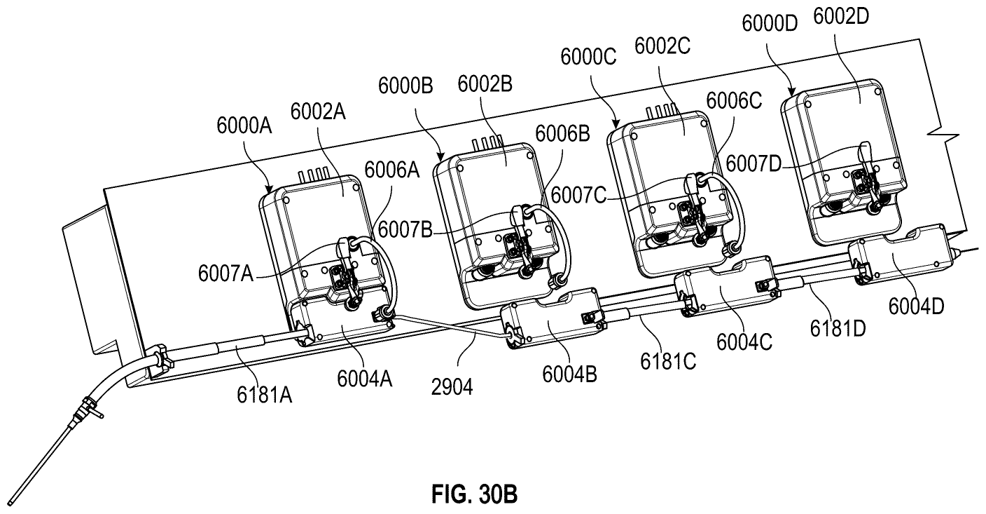

Certain embodiments of hub assemblies described herein, such as hub assembly (“hub”) 36 shown in FIG. 4, include a housing (e.g., housing 38) for coupling an interventional device thereto, components (e.g., roller 53 and 55) for directly coupling to and moving along a drive table, and magnet(s) (e.g., magnet 69) for magnetically coupling to a hub adapter across a sterile barrier. A hub (or hub assembly) can refer to a single assembly with a housing, or a hub (or hub assembly) can generally refer to an apparatus having two (or more) subassemblies (e.g., a first subassembly and a second subassembly). In some embodiments of a hub assembly having two subassemblies, a hub can refer to a first subassembly that can be configured to couple to and house an interventional device, and that may be removably attachable to a second subassembly (or mount) configured to magnetically couple to a hub adapter across a sterile barrier and move along a drive table. For example, as shown and described in relation to FIGS. 30A-30B below, a first subassembly or hub configured to couple to and house an interventional device may be removably attachable to a second subassembly or mount configured to magnetically couple to a hub adapter across a sterile barrier and move along a drive table. Such a hub and mount may together form a hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure).

An arrangement of a hub assembly having a hub that is releasably couplable to mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to a procedure catheter without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage.

An arrangement of a hub assembly having a hub that is releasably couplable to mount may also allow for a hub to be removed from a magnetically driven mount so that it can be used manually during a medical procedure (e.g., manually manipulated by a user to advance, retract, and/or rotate the hub and coupled interventional device). In such embodiments, the mount may maintain a magnetic connection with a hub adapter. In some such embodiments, an interventional device may be driven robotically for a portion of a procedure and manually for another portion of a procedure. In some embodiments, a plurality of interventional devices can be driven robotically for a portion of a procedure. Subsequently, a subset of the plurality of interventional devices may be driven manually by disconnecting the hubs of subset of interventional devices from their respective mounts. For example, in certain embodiments, a plurality of interventional devices including a guide catheter 2906 and one or more of a procedure catheter 2904, an access catheter 2902, and a guidewire 2907 can be driven until supra-aortic access is achieved, and the guide catheter 2902 is positioned within a desired ostium. Subsequently, one or more the procedure catheter 2904, the access catheter 2902, and the guidewire 2907 can decoupled from robotic drive system (e.g., by disconnecting their respective hubs from their respective mounts) and used to perform additional steps of the procedure. In other embodiments, one or more interventional devices may be controlled manually at the beginning of a procedure. Subsequently, the one or more interventional devices can be coupled to a robotic drive system (e.g., by connecting their respective hubs to mounts coupled to the robotic drive system) and used to perform additional steps of the procedure.

The robotic drive systems described herein can relate to various embodiments of systems that include a hub, or a hub and a mount, regardless of whether they are described in reference to a hub, or a hub and mount, unless explicitly indicated or indicated by context. In some embodiments, a mount may be magnetically coupled to hub adapter across a sterile barrier prior to the coupling a hub to the mount, for example, when preparing the drive table for a medical procedure.

As described herein, the hub assemblies can include intravascular devices that can access the vascular system of a patient via at least one artery and/or vein (e.g., the femoral artery) and be driven within the vascular system to perform a vascular procedure.

In some embodiments, a mount can be configured to provide fluid and/or vacuum to a hub. For example, in certain embodiments, a mount can include one or more fluid (e.g., saline and/or contrast) and/or vacuum channels to provide fluid and/or vacuum to the hub. A mount may include one or more robotically actuated valves for selectively placing one or more fluid and/or vacuum channels in fluid communication with the hub to provide fluid and/or vacuum thereto (e.g., to provide fluid and/or vacuum to a lumen of a catheter or to provide fluid and/or vacuum for flushing a hemostasis valve of the hub). A fluid connector (e.g., a tube) may be coupled to the mount and the hub to provide fluid communication therebetween.

A mount can be configured to transfer force and/or motion to the hub. For example, the mount may include an output member (e.g., a gear) that can communicate with an input member (e.g., a gear) of the hub to transfer force and/or motion thereto. In some embodiments, a gear train within the mount can be coupled with a gear train within the hub when the hub is coupled to the mount so that actuation of the gear train within the mount causes actuation of the gear train within the hub.

In some embodiments, a mount can be configured to transfer force and/or motion to the hub to cause rotation of an interventional device (e.g., a catheter or guidewire) within the hub. For example, the interventional device may be coupled to a gear train within the hub that can be actuated by a gear train within the mount when the hub is coupled to the mount.

A mount can be configured to transfer force and/or motion to the hub to cause an actuation of a valve within the hub (e.g., a hemostasis valve). For example, the valve can be actuated between various states, such as an opened state and a closed stated. For example, the valve may be coupled to a gear train within the hub that can be actuated by a gear train within the mount when the hub is coupled to the mount.

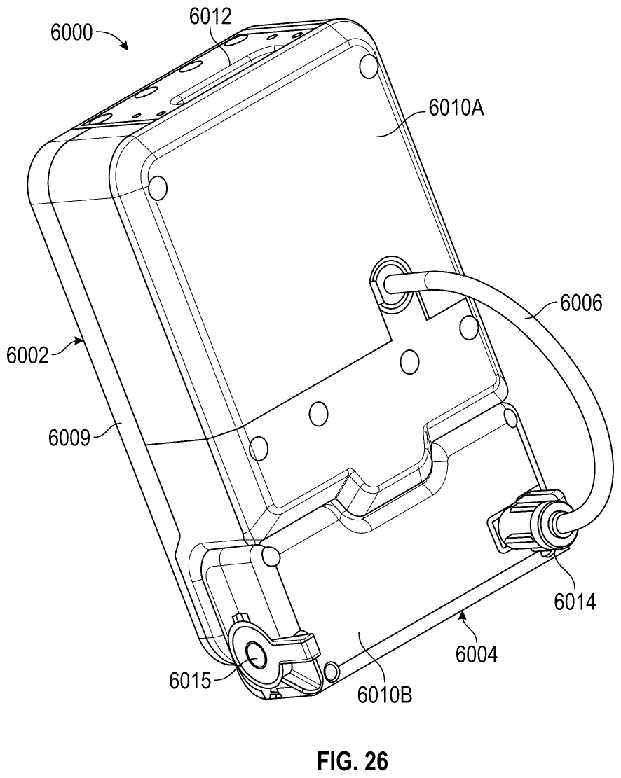

FIG. 26 illustrates a hub assembly 6000. The hub assembly 6000 may include any of the same or similar features and/or functions as the hubs described herein.

In some embodiments, the hub assembly 6000 can include a first subassembly, puck, or mount 6002 and a second subassembly or hub 6004. The mount 6002 can also be referred to as a catheter puck, a hub mount, and/or a first hub member. The mount 6002 can be configured to couple to and move along a drive table. The hub assembly 6000 can be configured to be positioned on a sterile side (e.g., a disposable equipment side) of a sterile barrier.

In some embodiments, the hub 6004 can be referred to as a second hub member. The hub 6004 may include or couple to an interventional device, such as a catheter or guidewire.

As described herein, in certain embodiments an interventional device may be coupled to a fluidics management system (e.g., to receive fluids such as contrast or saline, or for aspiration). In some embodiments, the mount 6002 can be coupled to the fluidics management system. In some embodiments, a fluidics connector 6006 can extend between and fluidly couple the mount 6002 and the hub 6004.

The mount 6002 can further include a first housing 6010A. The first housing 6010A can define one or more openings 6012 and a plurality of internal components described in greater detail below. The first housing 6010A can form an outer shell to protect the internal components of the mount 6002. The first housing 6010A can include at least one side shaped and/or dimensioned (e.g., having a contour) for receiving the hub 6004.

The one or more openings 6012 can provide access for fluidics and/or electrical connections into the mount 6002. In some embodiments, a contrast tube, a saline tube, and/or an aspiration tube may extend through the one or more openings 6012 into the mount 6002. Additionally, in some embodiments, a power line may extend through the one or more openings 6012 to provide electrical power into the mount 6002. The mount 6002 can be configured to receive an input from one or more active torque elements 5104A-5104C of the active torque subsystem 5102. In some embodiments, the inputs from the one of more active torque elements 5104A-5104C may be a magnetic rotary force as described herein. The mount 6002 can be configured to transmit one or more outputs to the hub 6004. In some embodiments, the mount 6002 may transform one or more rotary inputs of the one or more active torque elements 5104A-5104C into corresponding linear and/or rotary outputs. In some embodiments, the mount 6002 may be configured to translate linearly along a drive table (e.g., in response to linear movement of hub adapter within the drive table due to a magnetic coupling between mount 6002 and the hub adapter).

The hub 6004 can further include a second housing 6010B. The hub 6004 can include a lumen 6015 for receiving an interventional device therein. The hub 6004 can include a luer 6014. The hub 6004 can further include a plurality of internal components described in greater detail below. The second housing 6010B can form an outer shell to protect the internal components of the hub 6004. In some embodiments, the second housing 6010B may include at least one side shaped and/or dimensioned (e.g., having a contour) to correspond to shape of the first housing 6010A. For example, the contour of the second housing 6010B can correspond to the contour of the first housing 6010A of the mount 6002. The hub 6004 can be configured to receive one or more inputs from the mount 6002. The hub 6004 can be configured to transmit one or more outputs. In some embodiments, the hub 6004 may transform the outputs of the mount 6002 into corresponding linear and/or rotary motion of components within or coupled to the hub 6004 (e.g., the interventional device coupled to the hub and/or one or more fluidics components).

The fluidics connector 6006 can be a tubular body defining an interior lumen extending from one end of the fluidics connector 6006 to a second end of the fluidics connector 6006. In some embodiments, the fluidics connector may be configured to transport fluids between the mount 6002 and the hub 6004. For example, the fluidics connector 6006 may facilitate the flow of contrast, saline, bodily fluids, and/or air between the mount 6002 and the hub 6004. The fluidics connector 6006 can transport fluids from the mount 6002 to the hub 6004, or vice versa. The fluidics connector 6006 may form an airtight seal.

The hub 6004 may be removably coupled to the mount 6002. In some embodiments, the hub 6004 can be mounted to a mounting element defined by the mount 6002. The fluidics connector 6006 may be coupled to both the mount 6002 and the hub 6004. In some embodiments, the hub 6004 may be in fluid communication with the mount 6002 via the fluidics connector 6006. Accordingly, fluids may be transferred between the mount 6002 and the hub 6004 via the fluidics connector 6006.

Referring to FIGS. 27A-27E, the mount 6002 is described in greater detail.

FIG. 27A illustrates a front perspective view of the mount 6002. The mount 6002 can further include a base 6009. The mount 6002 can further include one or more output gears 6112A, 6112B. The mount can further include a platform 6018. The mount can further include one or more alignment pins 6022.

The first housing 6010A can include a plurality of openings through which the one or more output gears 6112A, 6112B can at least partially extend. The one or more output gears 6112A, 6112B can be configured to provide an output of the mount 6002 as an input to the hub 6004. In some embodiments, the rotary motion of the one or more output gears 6112A, 6112B may result in a movement of one or more components within the hub 6004 as described herein.

The platform 6018 can be configured to receive the hub 6004. The one or more alignment pins 6022 can be configured to engage with a bottom surface of the hub 6004 and prevent the hub 6004 from moving relative to the mount 6002.

FIG. 27B illustrates a bottom view of the mount 6002. As shown in FIG. 27B, the base 6009 of the mount can further include the passive torque elements 6104A-6104C of the passive torque subsystem 6102.

FIG. 27C illustrates a front perspective view of the interior of the mount 6002. The mount 6002 can further include one or more gear assemblies or gear trains 6105A, 6105B, 6105C. The mount can include a manifold 6024. The mount can include a valve 6028 (e.g., a three-way valve). The manifold 6024 can further include one or more fluidic channels 6026A, 6026B, 6026C, 6026D.

The one or more gear trains 6105A-6105C can be configured to provide an input to the hub 6004 and/or control a function of the mount 6002 (e.g., control the valve 6028). The one or more gear trains 6105A-6105C can include an input gear and an output gear. In some embodiments, the one or more gear trains 6105A-6105C can further include one or more intermediate gears. The one or more gear trains 6105A-6105C can be operatively coupled to the one or more passive torque elements 6104A-6104C. In some embodiments, the input gears of the one or more gear trains 6105A-6105C may extend from a corresponding passive torque element 6104A-6104C through the base 6009. Accordingly, the rotation of the passive torque elements 6104A-6104C can result in a corresponding rotational motion of the input gears of the one or more gear trains 6105A-6105C.

In some embodiments, the one or more gear trains 6105A-6105C may include a first gear train 6105A, a second gear train 6105B, and a third gear train 6105C. The first gear train 6105A can be configured to transmit a drive motion from the first passive torque element 6104A to the hub 6004 for controlling a motion of an interventional device and/or guidewire. In some embodiments, the first gear train 6105A can be configured to control a roll motion or rotational motion of an interventional device within the hub 6004.

The second gear train 6105B can be configured to translate a drive motion from the second passive torque element 6104B to the valve 6028. In some embodiments, the second gear train 6105B can be configured to rotate the valve 6028 between fluidic configurations.

The third gear train 6105C can be configured to transmit a drive motion from the third passive torque element 6104C to the hub 6004 for controlling a motion of an interventional device. In some embodiments, the third gear train 6105C can be configured to control an activation of a hemostasis valve within the hub 6004.

The manifold 6024 can be configured to receive and redistribute the fluidics within the mount 6002 as described in greater detail below. The manifold 6024 may include one or more fluidic channels. In some embodiments, the manifold 6024 can be configured to receive and/or transport saline and/or contrast to the valve 6028. In some embodiments, the manifold 6024 can be configured to aspirate the three-way valve 6028 to flush the three-way valve 6028 and/or receive bodily fluids from the three-way valve 6028.

In some embodiments, the manifold 6024 may include a first fluidic channel 6026A, a second fluidic channel 6026B, and a third fluidic channel 6026C. The first fluidic channel 6026A, the second fluidic channel 6026B, and the third fluidic channel 6026C can be configured to receive and/or transport one of saline, contrast, and/or aspiration. For example, the first and second fluidic channels 6026A and 6026B can be inlets to the valve 6028 for providing fluidics (e.g., saline and/or contrast) to the hub 6004 and the third fluidic channel 6026C can be configured to provide saline, contrast, and/or aspiration to the luer 6014.

In some embodiments, the manifold 6024 can further include an electrical channel 7022. The electrical channel 7022 may be a groove or channel formed within the manifold 6024. The electrical channel 7022 may be used to guide electrical cables or wires across the mount 6002. In some embodiments, the cables or wires extending across the electrical channel 7022 may be used to electrically connect to one or more sensors.

The valve 6028 can be a rotational valve for selectively controlling the flow of fluidics through the mount 6002. As described above, the valve 6028 can be controlled by the passive torque element 6104B. The valve 6028 may be a stopcock. The valve 6028 may include a plurality of openings having interconnected interior channels and/or lumens. In some embodiments, the valve 6028 can be a three-way stopcock having a first port, a second port, and a third port. The ports can also be referred to as openings. In some embodiments, the configuration or rotational position of the valve 6028 may selectively control the flow of fluidics in the mount 6002 as described in greater detail below.

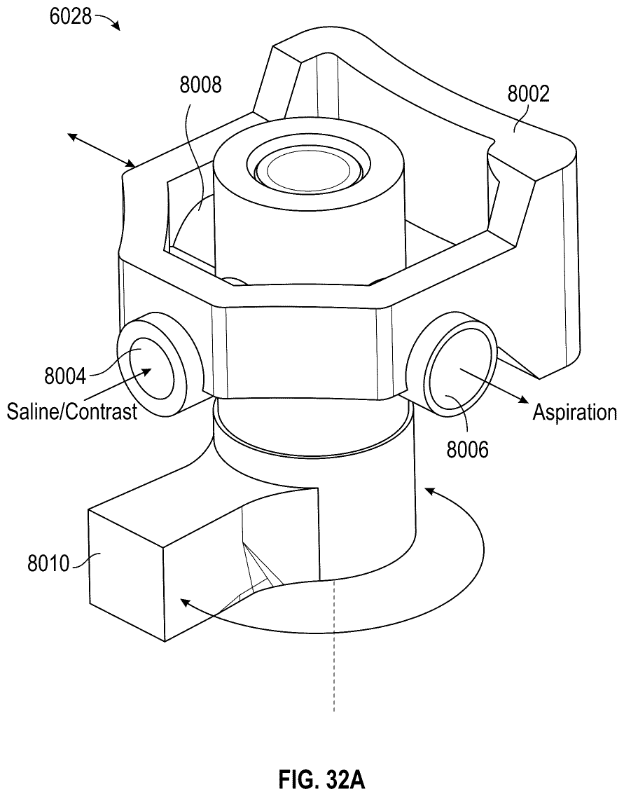

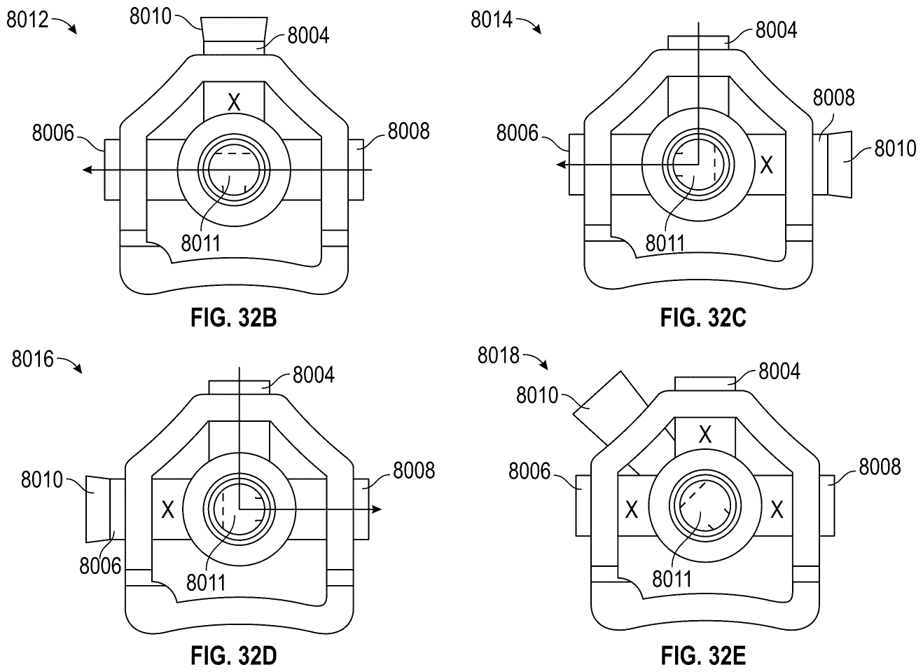

In some embodiments, the first port can be coupled to a source of contrast media and/or a source of saline and the second port can be coupled to a vacuum or aspiration source. In certain embodiments, the third port can be coupled to the interventional device to provide contrast and/or saline or apply vacuum thereto. In some embodiments, the third port can be coupled to a port of a hemostatic valve coupled to an interventional device to provide contrast and/or saline or apply vacuum thereto. The valve 6028 can include a stopcock valve portion or stopcock control that can be rotated to selectively provide or prevent fluid communication of the ports with one another (e.g., via fluid passages extending through the stopcock control). In certain embodiments, the stopcock control can be rotated to a first position in which the second port is in fluid communication with the third port as described further in FIG. 32B below. In certain embodiments, the stopcock control can be rotated to a second position in which the first port is in fluid communication with the first port as described further in FIG. 32C below. In certain embodiments, the stopcock control can be rotated to a third position in which the first port is in fluid communication with the third port as described further in FIG. 32D below. In certain embodiments, the stopcock control can be rotated to a fourth position in which none of the ports are in fluid communication as described further in FIG. 32E below. In certain embodiments, the stopcock control can be rotated to a fifth position in which the ports are each in fluid communication with one another.

FIG. 27D illustrates another front perspective view of the internal components of the mount 6002. As shown in FIG. 27D, the manifold 6024 can include the third fluidic channel 6026C. The third fluidic channel 6026C may be in fluid communication with the fluidics connector 6006 and the three-way valve 6028. In some embodiments, the third fluidic channel 6026C can be connected to the third port and/or opening of the valve 6028 reserved as a pathway to the hub 6004.

FIG. 27E illustrates a top view of the one or more gear trains 6105A-6105C of the mount 6002.

The gear train 6105A can include an output gear 6112A. In some embodiments, the output gear 6112A can be configured to provide an output of the mount 6002 as an input to the hub 6004. In some embodiments, the output gear 6112A can be configured to drive movement of one or more components within the hub 6004 when the hub 6004 is coupled to the mount 6002. For example, in certain embodiments, the output gear can be configured to drive rotation of an interventional device coupled to the hub 6004. For example, rotation of the plurality of magnets of the passive torque element 6104A can cause rotation of the interventional device via rotation of the output gear 6112A.

In some embodiments, the gear train can include an input gear 6108A. In certain embodiments, the input gear 6108A may be part of or coupled to the passive torque element 6104A (e.g., coupled with the one or more magnets and/or the magnet support element of the passive torque element 6104A). In certain embodiments, rotation of the input gear 6108A (e.g., in response to rotation of the passive torque element 6104A) can be configured to cause rotation of the output gear 6112A. In certain embodiments, the gear train 6105A may include one or more intermediate gears 6109A and 6110A.

The input gear 6108A can be configured to couple with and rotate intermediate gear 6109A when the one or more magnets of the passive torque element 6104A are rotated. In some embodiments, the input gear 6108A and the intermediate gear 6109A can be miter gears or bevel gears. In some embodiments, the intermediate gear 6109A can be rotationally coupled with the intermediate gear 6110A. The intermediate gear 6110A can couple to an output gear 6112A of the mount 6002. In some embodiments, the intermediate gear 6110A can be fixedly coupled with the output gear 6112A. For example, the base of the output gear 6112A can be fixedly coupled to the top of the intermediate gear 6110A. The intermediate gear 6110A and the output gear 6112A may be pinion gears. Rotation of the plurality of magnets of the passive torque element 6104A can cause rotation of the interventional device via rotation of the gears of the gear train 6105A.

The gear train 6105B can include an output gear 6110B. The output gear 6110B can be coupled (e.g., rotationally) with the valve 6028 (e.g., coupled with the stopcock control) to adjust the configuration of the valve 6028 to provide or prevent fluid communication between the ports of the valve 6028 as described herein. The stopcock control can be rotated in response to rotation of the output gear 6110B to provide or prevent fluid communication between the ports of the valve 6028.

In some embodiments, the gear train can include an input gear 6108B. In certain embodiments, the input gear 6108B may be part of or coupled to the passive torque element 6104B (e.g., coupled with the one or more magnets and/or the magnet support element of the passive torque element 6104B). In certain embodiments, rotation of the input gear 6108B (e.g., in response to rotation of the passive torque element 6104B) can be configured to cause rotation of the output gear 6110B. In certain embodiments, the gear train 6105B may include one or more intermediate gears 6109B.

The input gear 6108B can be configured to couple with and rotate intermediate gear 6109B when the one or more magnets of the passive torque element 6104B are rotated. The intermediate gear 6109B can be further configured to rotate output gear 6110B. The input gear 6108B, the intermediate gear 6109B, and the output gear 6110B can be pinion gears. Rotation of the plurality of magnets of the passive torque element 6104B can cause rotation of the stopcock control via rotation of the gears of the gear train 6105B (e.g., the first gear 6108B and the second gear 6109B.

The gear train 6105C can include an output gear 6112B. In some embodiments, the output gear 6112B can be configured to provide an output of the mount 6002 as an input to the hub 6004. In some embodiments, the output gear 6112B can be configured to drive movement of one or more components within the hub 6004 when the hub 6004 is coupled to the mount 6002. For example, in certain embodiments, the output gear 6112B can be configured to control a hemostasis valve of the hub 6004 (e.g., to open and close the hemostasis valve).

In some embodiments, the gear train 6105C can include an input gear 6108C. In certain embodiments, the input gear 6108C may be part of or coupled to the passive torque element 6104C (e.g., coupled with the one or more magnets and/or the magnet support element of the passive torque element 6104C). In certain embodiments, rotation of the input gear 6108C (e.g., in response to rotation of the passive torque element 6104C) can be configured to cause rotation of the output gear 6112B. In certain embodiments, the gear train 6105C may include one or more intermediate gears 6109C and 6110C.

The input gear 6108C can be configured to couple with and rotate intermediate gear 6109C when the one or more magnets of the passive torque element 6104C are rotated. In some embodiments, the input gear 6108C and the intermediate gear 6109C can be miter gears or bevel gears. In some embodiments, the intermediate gear 6109C can be rotationally coupled with the intermediate gear 6110C. The intermediate gear 6110C can couple to an output gear 6112B of the mount 6002. In some embodiments, the intermediate gear 6110C can be fixedly coupled with the output gear 6112B. For example, the base of the output gear 6112C can be fixedly coupled to the top of the intermediate gear 6110C. The intermediate gear 6110C and the output gear 6112C may be pinion gears. Rotation of the plurality of magnets of the passive torque element 6104C can cause a change in configuration of the hemostasis valve via rotation of the gears of the gear train 6105C.

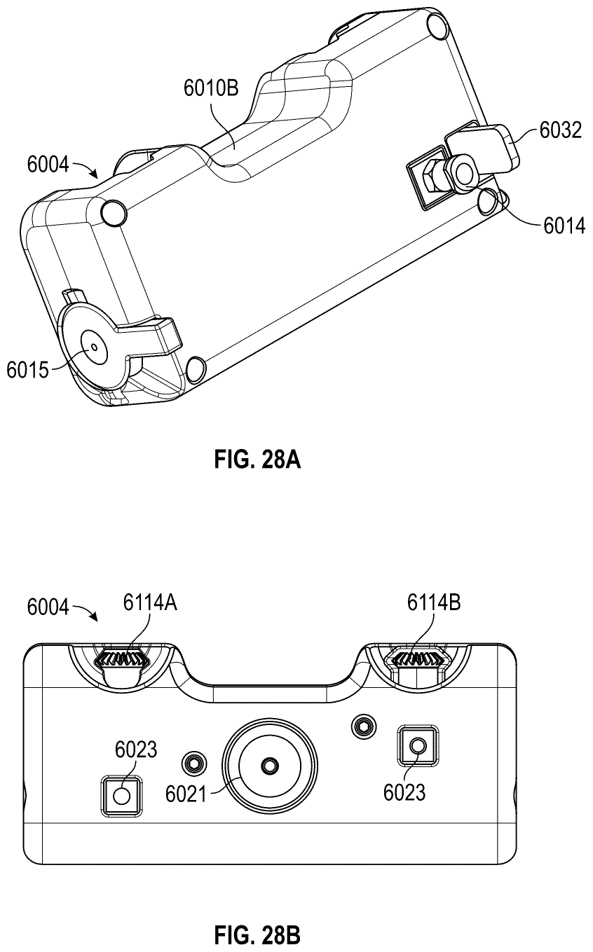

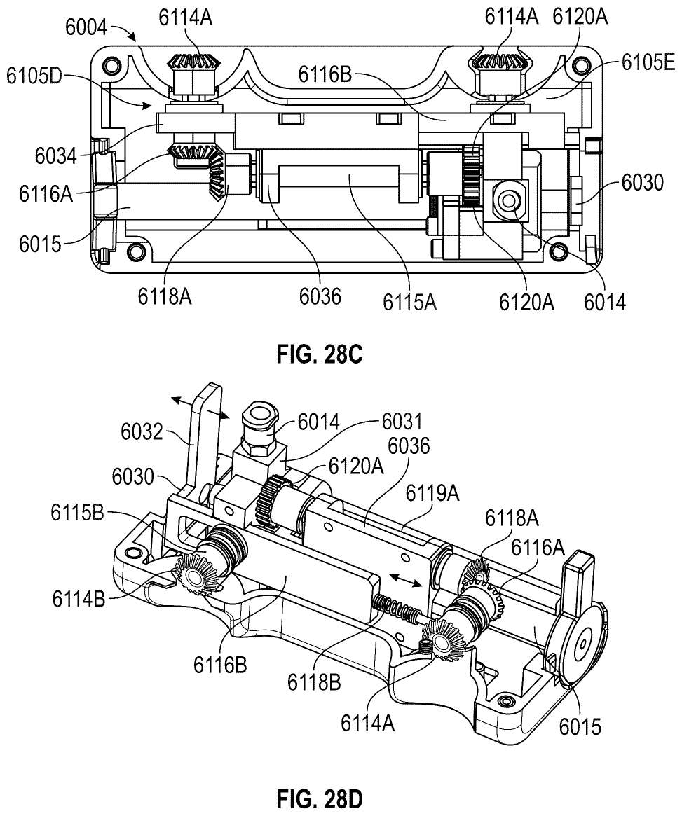

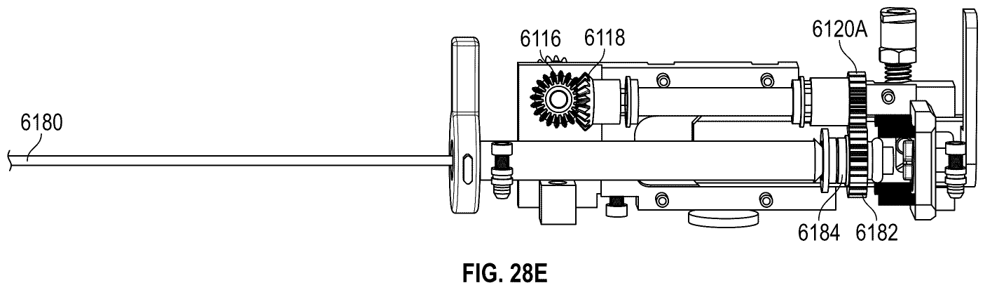

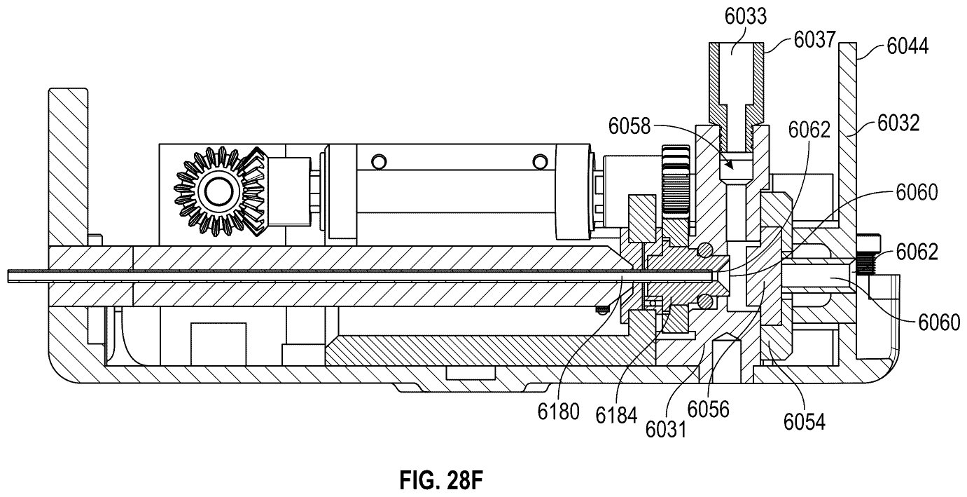

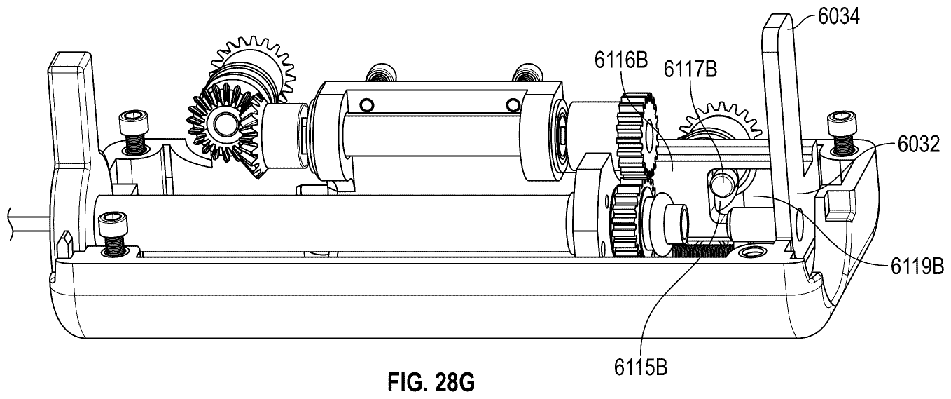

Referring to FIGS. 28A-28D, the hub 6004 is described in greater detail.

FIG. 28A illustrates a front perspective view of the hub 6004. As described herein, the hub 6004 can include a housing 6010B, a luer 6014, an interventional device lumen 6015, and a plunger 6032.

As described herein, an interventional device, such as a catheter or guidewire, can be fixedly coupled to the hub 6004 so that movement of the hub 6004 results in movement of the interventional device. A proximal end of the interventional device can be positioned within the interventional device lumen 6015 and extend out of the distal end thereof. The interventional device lumen 6015 can extend through the longitudinal length of the hub 6004. In certain embodiments, one or more additional interventional devices can be received within a proximal end of the interventional device lumen 6015 and the interventional device coupled to the hub 6004 and move relative to the hub 6004 and interventional device coupled to the hub 6004.

The second housing 6010B can include a plurality of openings through which the one or more gears can at least partially extend. The one or more gears can be configured for receiving an output of the mount 6002 and/or provide an input to the hub 6004. In some embodiments, the rotary motion of the one or more gears may result in a movement of the interventional device coupled to the hub 6004. In some embodiments, the rotary motion of the one or more gears may result in a change in configuration of a hemostasis valve of the hub 6004.

The luer 6014 can be a fluidics input into the hub 6004 or a fluidics outflow from the hub 6004. The luer 6014 can receive the fluidics connector 6006.

FIG. 28B illustrates a bottom view of the hub 6004. As shown in FIG. 28B, the hub 6004 can include an input gear 6114A. The input gear 6114A can engage with the output gear 6112A of the mount 6002 so that rotation of the output gear 6112A causes rotation of the input gear 6114A. The hub 6004 can include an input gear 6114B. The input gear 6114B can engage with the output gear 6112B of the mount 6002 so that rotation of the output gear 6112B causes rotation of the input gear 6114B.

In some embodiments, the hub can include and one or more alignment holes 6023. The alignment holes 6023 can be configured to engage with the alignment pins 6022 of the mount 6002 when the hub is coupled to the mount 6002. The alignment holes 6023 can be configured to engage with input pins 6022 to prevent the hub 6004 from moving relative to the mount 6002. Preventing relative motion may advantageously ensure proper gear engagement between a hub and a mount.

FIGS. 28C-28G illustrate the internal components of the hub 6004. As shown, the hub 6004 can include a hemostasis valve 6030. The hub 6004 can include a gear train 6105D. The hub 6004 can include a gear train 6105E. The hub 6004 can include a first support surface 6034. The hub 6004 can include and a second support surface 6036.

The gear train 6105D can include the input gear 6114A. The gear train 6105A can include an output gear 6120A. The output gear 6120A can be coupled to a gear 6182 coupled to an interventional device 6180. In some embodiments, the gear 6182 can be coupled to a proximal end termination 6184 of the interventional device 6180. In some embodiments, the interventional device 6180 can be a guidewire or an access catheter. In some embodiments, the output gear 6120A can be a worm gear. A worm gear may prevent back driving of the interventional device, for example, in response to torsional strain produced while navigating tortuous anatomy.

In some embodiments, the gear train 6105D can include one or more intermediate gears. For example, in some embodiments, the gear train 6105D can include a pivoting gear 6116A and a pivoting gear 6118A. In some embodiments, the gear train 6105D can include a rotating body 6115A.

The first pivot gear 6116A can be fixedly attached to the input gear 6114A. In some embodiments, a shaft can extend from the input gear 6114A to the first pivot gear 6116A. The shaft extending between the input gear 6114A and the first pivot gear 6116A can be supported by the first support surface 6034. In some embodiments, the shaft can extend through an opening in the first support surface 6034. The second pivot gear 6118A can be fixedly attached to the output gear 6120A. In some embodiments, a shaft can extend from the second pivot gear 6118A to the output gear 6120A. In some embodiments, the shaft can be supported by a second support surface 6034. In some embodiments, the second pivot gear 6118A is oriented 90 degrees from the first pivot gear 6116A. In some embodiments, the first and second pivot gears 6116A, 6118A can be miter gears. The input gear 6114A can receive a rotary input from the output gear 6112A of the mount 6002. The first pivot gear 6116A can rotate with the input gear 6114A. The first pivot gear 6116A can mesh with the second pivot gear 6118A to pivot the rotational motion 90 degrees. The rotational motion of the second pivot gear 6118A can be transmitted along the shaft to the output gear 6120A. The output gear 6120A can mesh with an input gear of the hemostasis valve. Accordingly, the rotational input of the input gear 6114A from the output gear 6112A can be transmitted to an interventional device along the fourth gear train 6105D.

The fifth gear train 6105E can further include the input gear 6114B, a rotating body 6115B having a pin 6117B, a yoke or slider 6116B, and a spring 6118B.

The hemostasis valve 6030 can further include the plunger 6032. The hemostasis valve 6030 can have a body 6031 having a fluid port 6033 that can be placed in fluid communication with a fluidics system for the delivery of fluids (e.g., saline and/or contrast media) to the interventional device and/or for aspiration of fluids from the interventional device. For example, in some embodiments, the fluid port 6033 can be in fluid communication with the three-way valve 6028. The fluid port 6033 can include a luer connector 6037 (e.g., a female luer connector) configured to couple to the luer 6014 (e.g., a male luer connector) of the fluidics connector 6006.

The body 6031 can receive and/or be coupled to a proximal end termination 6184 of an interventional device 6180. The body 6031 can be coupled to a cap 6054. The cap 6054 can be coupled to a seal 6056. The seal 6056 can be at least partially positioned within the body 6031. In certain embodiments, the body 6031 can at least partially define a channel 6058 between the fluid port 6033 and the interventional device 6180. In certain embodiments, the body 6031 can at least partially define a channel 6060 between a proximal opening 6062 of the hemostasis valve 6030 and the interventional device 6180. In certain embodiments, the plunger 6032 may at least partially define the channel 6060. In certain embodiments, the proximal opening 6062 can be an opening of the plunger 6032.

The hemostasis valve 6030 can be configured to receive a more proximal interventional device therethrough. For example, the hemostasis valve 6030 can include the channel 6060 for receiving a more proximal interventional device therethrough. In some embodiments the channel 6060 may be coaxial with the interventional device lumen 6015.

The hemostasis valve 6030 can be actuatable between various states to allow for and/or restrict movement of the interventional device therethrough and to allow for and/or prevent fluid flow therethrough. For example, in some embodiments, the hemostasis valve may actuate between an open state of the seal 6056 and a closed state of the seal 6056. In some embodiments, the hemostasis valve 6030 can be actuatable between a first fully open state, a second partially opened (low sealing force state) for sealing around an interventional device but permitting sliding movement of the interventional device, a third state for sealing around an interventional device for high pressure management, and a fourth completely closed state in the absence of any interventional devices extending therethrough. In certain embodiments, the passive torque element 6104C can be configured to actuate the hemostasis valve 6030 through one or more of the foregoing states. In some embodiments, the gear 6112B of the mount 6002 and the input gear 6114B of the hub 6004 can be configured to actuate the hemostasis valve 6030 through one or more of the foregoing states.

In some embodiments, the hemostasis valve 6030 can be actuated between various states in response to movement of the gear train 6105E. For example, the plunger 6032 can be actuated by movement of the slider 6116B to transition the seal 6056 of the hemostasis valve 6030 between various states. In some embodiments, the slider 6116B is part of the plunger 6032. In other embodiments, the slider 6116B is a separate component coupled to the plunger 6032.

The input gear 6114B can be coupled to the rotating body 6115B. The rotating body can rotate so that the pin 6117B moves within a slot 6119B of the slider 6116B to cause linear motion of the slider 6116B, resulting in linear motion of the plunger 6032. The spring 6118B can be linearly depressed as the slider 6116B is linearly actuated in the direction of the spring 6118B. The spring 6118B can act to bias the slider 6116B to a first position. Accordingly, the fifth gear train 6105E can drive the hemostasis valve 6030. Additionally and/or alternatively, the plunger 6032 of the hemostasis valve 6030 can be manually actuated by manually depressing the tab 6044 extending vertically from the hub 6004. The tab 6044 can be part of or coupled to the plunger 6032.

In certain embodiments, the hub of FIGS. 26-28D may be used for interventional devices for which rotation and fluidics may be desired (e.g., an access catheter, such as access catheter 2902.

In some embodiments, the hub 6004 can be detached from the mount 6002 by releasing a locking mechanism. In some embodiments, the locking mechanism may include a depressible feature to disengage the locking mechanism from the hub 6004. For example, in some embodiments, the depressible feature may be a button or lever. In some embodiments, the locking mechanism may be a toggle clamp, described in greater detail below, coupled to the mount 6002 and removably engageable with the hub 6004. Additionally and/or alternatively, the mount may include one or more clips. The one or more clips may be configured to latch onto a corresponding hub to keep the hub in place. Securing the hub to the mount may advantageously provide proper gear engagement. In some embodiments, after detachment, a different hub 6004 (e.g., coupled to a different interventional device of the same type or an interventional device of a different type) can be coupled to the mount 6002.

In some embodiments, rotation of an interventional device (e.g., a guide catheter, such as guide catheter hub 2914, or a procedure catheter, such as procedure catheter 2904) may not be desired. In some such embodiments, the mount 6002 may not include the output gear 6112A and/or the gear train 6105A. Additionally or alternatively, in some embodiments in which rotation of an interventional device is not desired, the hub 6004 may not include the input gear 6114A and/or gear train 6105A. Alternatively, in some embodiments, the gear train 6105A may be present, but not configured to rotate the interventional device. In certain embodiments, it may be beneficial for the mount 6002 to include the output gear 6112A so that the mount 6002 can be coupled to a hub 6004 configured to rotate an interventional device or a hub 6004 configured not to rotate an interventional device.

In some embodiments, manipulation of a hemostasis valve may not be desired. For example, a hub 6004 coupled to a guidewire may not be configured to receive additional interventional device proximally therethrough and may not be configured to couple to fluidics. Accordingly, such a hub may not include a hemostasis valve. In such embodiments, the mount 6002 may not include the output gear 6112B and/or the gear train 6105C. Additionally or alternatively, in some embodiments in which rotation of an interventional device is not desired, the hub 6004 may not include the input gear 6114B and/or gear train 6105B. Alternatively, in some embodiments, the gear train 6105B may be present, but not configured to cause a change in configuration of a hemostasis valve. In certain embodiments, it may be beneficial for the mount 6002 to include the output gear 6112A so that the mount 6002 can be coupled to a hub 6004 having a hemostasis valve and hub 6004 without a hemostasis valve.

In some embodiments, mounts 6002 of different types may be used in a single interventional device assembly. For example, in some embodiments, a mount 6002 having an output gear 6112B may be coupled to a hub 6004 for a guide catheter, a mount 6002 having an output gear 6112B may be coupled to a hub 6004 for a procedure catheter, a mount 6002 having an output gear 6112A and an output gear 6112B may be coupled to a hub 6004 for an access catheter, and a mount 6002 having an output gear 6112A may be coupled to a hub 6004 for a guidewire.

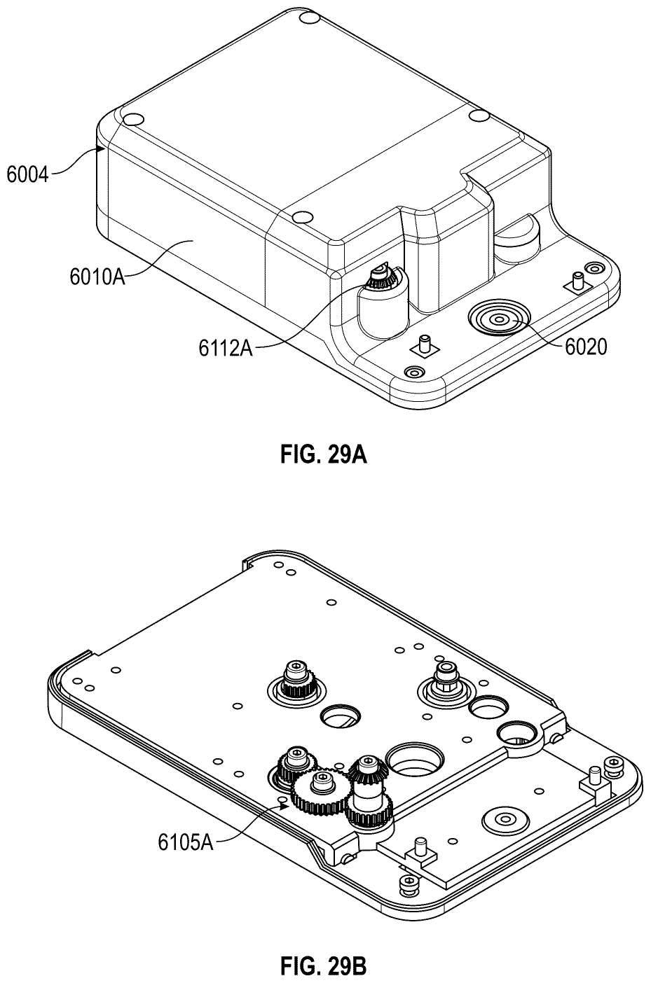

FIGS. 29A-29B illustrate an embodiment of the mount 6002 having an output gear 6112A but not an output gear 6112B. In some embodiments, the mount 6002 may comprise only a first gear train 6105A. The first gear train 6105A may be the same as the first gear train 6105A described above. The embodiment shown in FIGS. 29A-29B can be useful for driving interventional devices, such as guidewires, that do not require fluidics. Accordingly, costs may be reduced by only including the requisite gear trains.

FIGS. 30A-30B illustrate a plurality of hub assemblies 6000A-D, each hub assembly 6000 connected to a corresponding interventional device. The plurality of hub assemblies 6000A-D can include a first hub assembly 6000A having a first mount 6002A and a first hub 6004A, a second hub assembly 6000B having a second mount 6002B and a second hub 6004B, a third hub assembly 6000C having a third mount 6002C and a third hub 6004C, and a fourth hub assembly 6000D having a fourth mount 6002D and a fourth hub 6004D.

In certain embodiments, the first hub 6004A can be coupled to a guide catheter. In certain embodiments, the second hub 6004B can be coupled to a procedure catheter. In certain embodiments, the third hub 6004C can be coupled to an access catheter. In certain embodiments, the fourth hub 6004D can be coupled to a guidewire.

One or more of the hubs 6004A-D can include a valve, such as a hemostasis valve, to allow interventional devices to advance therethrough. For example, in certain embodiments, the hub 6004A can include a hemostasis valve to allow one or more interventional devices coupled to hubs 6004B-D to advance therethrough to facilitate concentric arrangement of the interventional devices.

Such hub assemblies 6000A-D may allow for a hub 6004A-D to be removed from a mount 6002A-D, for example, so that a different hub can be coupled to the same mount 6002A-D. In some embodiments, a manually driven interventional device can be coupled to the same mount 6002A-D so that it can be robotically driven. In other embodiments, mount 6002A-D may initially have no interventional device coupled thereto and a manually driven interventional device can be coupled to the mount 6002A-D so that it may be robotically driven. Such an arrangement can allow for the replacement of a hub 6004A-D with a different hub having a different interventional device coupled thereto without breaking a magnetic connection with a hub adapter. For example, such an arrangement may allow for the third hub 6004C coupled to an access catheter to be removed from the third mount 6002C and replaced with a hub coupled to a procedure catheter without breaking a magnetic connection with a hub adapter.

In some embodiments, a hub 6004A-D can be removed from a first mount 6002A-D and coupled to a second mount 6002A-D that is distal or proximal to the first mount, for example, to provide a different set of interventional devices or different arrangement of interventional devices for a portion of a procedure in comparison to a previous portion of the procedure.

For example, in some embodiments, an access assembly of interventional devices may be coupled to the drive table for a first part of a medical procedure and a procedure assembly of interventional devices can be coupled to the drive table for a second part of the medical procedure. In some embodiments, a hub 6004A coupled to a guide catheter can be coupled to mount 6002A, a hub 6004B coupled to an insert or access catheter (e.g., a 5 Fr insert or access catheter) can be coupled to mount 6002B, and a hub 6004C coupled to a guidewire (e.g., a guidewire having a 0.035 in diameter) can be coupled to mount 6002C and used to achieve supra-aortic access. While achieving supra-aortic access, the mount 6002D may have no hub coupled thereto. After supra-aortic access is achieved, the hub 6004B and hub 6004C may be removed. Subsequently, a hub coupled to a procedure catheter can be coupled to mount 6002B, a hub coupled to an insert or access catheter (e.g., an access catheter having an inner diameter of 0.035 in) can be coupled to the mount 6002C, and a hub coupled to a guidewire (e.g., a microwire having a diameter of 0.014 in) can be coupled to the mount 6002D, and a procedure, such as aspiration of a clot, may be performed.

In some embodiments, for example when performing a thrombectomy on a distal, medium vessel occlusion (DMVO), it may not be possible to reach the clot with certain procedure catheters, such as a procedure catheter having a 0.071 inch inner diameter). In such embodiments, a catheter having a smaller outer diameter may be used to reach and aspirate the clot. For example, when an initial arrangement of interventional devices includes a first hub 6004A coupled to a guide catheter, a second hub 6004B coupled to a procedure catheter, a third hub 6004C coupled to an access catheter (e.g., a 5 Fr access catheter), and a fourth hub 6004D coupled to a guidewire, the hub 6004C may be removed and replaced with a hub coupled to a smaller catheter (e.g., a 0.035 inch inner diameter catheter) to navigate to (e.g., through the procedure catheter) and aspirate the clot. In other embodiments, the smaller catheter may instead to navigated to the clot manually.

In some embodiments, hub assemblies 6000A-D may allow for a hub 6004A-D to be removed from a mount 6002A-D to facilitate performance of a procedure through the hub assemblies 6000A-D having hubs 6004A-D coupled to mounts 6002A-D (e.g., by providing more working space along the drive table). For example, as described herein, in certain embodiments, an access catheter and/or guidewire can be withdrawn from a procedure catheter prior to aspiration using the procedure catheter. In certain embodiments, the access catheter hub 6004C and/or guidewire hub 6004D can be removed from their respective mounts 6002C and 6002D before, after, or while withdrawing the access catheter and/or guidewire from the procedure catheter prior to aspiration using the procedure catheter. In some such embodiments, the guide catheter hub 6004A and procedure catheter hub 6004B may remain coupled to their respective mounts 6002A and 6002B. In other embodiments, the procedure catheter hub 6004B or the procedure catheter hub 6004B and the guide catheter hub 6004A can be uncoupled from their respective mounts 6002A and 6002B prior to performing the aspiration procedure.

In some embodiments, hub assemblies 6000A-D may allow for a hub 6004A-D to be removed from a mount 6002A-D to facilitate a path for one or more manual devices to be inserted through a more distal hub assembly. For example, in some embodiments, when an initial arrangement of interventional devices includes a first hub 6004A coupled to a guide catheter, a second hub 6004B coupled to a procedure catheter, a third hub coupled to an access catheter, and a fourth hub 6004D coupled to a guidewire, the hub 6004B, hub 6004C, and hub 6004D can be removed. Following removal, a stent may be manually navigated through the guide catheter for manual tandem lesion stent placement. In some such embodiments, the stent is navigated through the guide catheter while the hub 6004A is coupled to the mount 6002A. In other embodiments, the hub 6004A may be removed from the mount 6002A while the guide catheter is positioned within the vasculature of the patient, and the stent can then be navigated through the guide catheter.

Alternatively, one or more of the hub assemblies 6000A-D can be moved proximally towards a proximal end of a drive table to withdraw their corresponding interventional devices to facilitate a path for one or more manual devices to be inserted through a more distal hub assembly.

In some embodiments, hub assemblies 6000A-D may allow for a hub 6004A-D to be removed from a mount 6002A-D so that the hub 6004A-D may be used separately from the mount 6002A-D (e.g., for a manual procedure). Such an arrangement may allow for a hub 6004A-D to be removed from a magnetically driven mount 6002A-D so that the hub 6004A-D can be used manually during a medical procedure (e.g., manually manipulated by a user to advance, retract, and/or rotate the hub 6004A-D and coupled interventional device).

In some embodiments, an interventional device may be driven robotically for a portion of a procedure and manually for another portion of a procedure. For example, as shown in FIG. 30A, in some embodiments, one or more of the hubs 6004A-D can be coupled to and physically connected with a corresponding mount 6002A-D, during at least a portion of a procedure. As further illustrated in FIG. 30A, the interventional devices may be in a stacked and/or nested arrangement.

In some embodiments, a subset of the plurality of interventional devices may be driven manually by disconnecting one or more of the hubs 6004A-D from their respective mounts 6002A-D during a portion of a procedure. For example, FIG. 30B depicts the hub 6004A coupled to the mount 6002A, the hub 6004B disconnected from the mount 6002B, the hub 6004C disconnected from the mount 6002C, and the hub 6004D disconnected from the mount 6002D.

In certain embodiments, a plurality of interventional devices can be robotically driven to a desired position within the vasculature of a patient during a first portion of a procedure (e.g., via a coupling between their respective hubs 6004A-D and robotically driven mounts 6002A-D). Subsequently, all of the interventional devices or a subset of the interventional devices can be decoupled from robotic drive system by disconnecting their respective hubs 6004A-D from their respective mounts 6002A-D while portions of the interventional devices are positioned within the vasculature of the patient. For example, in certain embodiments, a plurality of interventional devices can be robotically driven to achieve supra-aortic access. After supra-aortic access is achieved all or a subset of the interventional devices can be decoupled from the robotic drive system by disconnecting their respective hubs 6004A-D from their respective mounts 6002A-D, and additional procedure steps may be performed manually.

For example, in certain embodiments, a first hub assembly 6000A having a first hub 6004A coupled to a guide catheter 2906, a second hub assembly 6000B having a second hub 6004B coupled to a procedure catheter 2904, a third hub assembly 6000C having a third hub 6004C coupled to an access catheter 2902, and a fourth hub assembly 6000D having a fourth hub 6004D coupled to a guidewire 2907 can be driven until supra-aortic access is achieved, and the guide catheter 2906 is positioned within a desired ostium. Subsequently, one or more of the hubs 6004B-D can be decoupled from its respective mounts 6002B-D, so that one or more of the procedure catheter 2904, the access catheter 2902, and the guidewire 2907 can be used to perform additional steps of the procedure manually as shown in FIG. 30B. For example, one or more of the procedure catheter 2904, the access catheter 2902, and the guidewire 2907 can be manually advanced further distally in the anatomy. In other embodiments, for example, the access catheter 2902 and guidewire 2907 can be withdrawn for the procedure catheter 2904, before or after detachment of the hub 6004B from its mount 6002B, and one or more additional manual interventional devices, such as a stent retriever and/or a stent retriever delivery microcatheter, can be inserted into the procedure catheter to perform additional steps of a procedure manually.

In other embodiments, the hub 6004A can be detached from the mount 6002A either by itself (e.g., after withdrawal of the procedure catheter 2904, access catheter 2902, and guidewire 2907 from the guide catheter 2906) or in addition to detachment of the hub 6004B from the mount 6002B (or detachment of additional hubs from their respective mounts) for performing additional procedure steps manually (e.g., by manually manipulating the hub 6004A and/or by inserting additional manual interventional devices through hub 6004A and the guide catheter 2906).

As further illustrated in FIG. 30B, one or more interventional devices may remain in a stacked and/or nested configuration after being decoupled. Portions of the interventional devices may remain within the vasculature and/or within the lumens of adjacent interventional devices while being decoupled from the robotic drive system.

The hubs 6004A-D may include surfaces configured to both be supported and manipulated by a corresponding mount 6002A-D and/or by a human operator. Accordingly, when the one or more hubs 6004A-D are decoupled from a corresponding one or more mounts 6002A-D, the corresponding one or more interventional devices may be positioned and movable within the patient's body by manual manipulation of the hubs 6004A-D (e.g., by a user grasping, rotating, and/or axially moving the hubs).

In some embodiments, as shown in FIG. 30A, each hub 6004A-C may be in fluid communication with a corresponding mount 6002A-C via a conduit 6006A-C. Each mount 6002A-C may be in communication with a fluidics system, which may provide fluids (e.g., saline, contrast, and/or therapeutic agents) and/or vacuum. The conduit 6006A-C can connect the catheters coupled to the hubs 6004A-C with the fluidics system to provide fluids or vacuum to the catheters. In some embodiments, one or more hubs may not be in fluid communication with a corresponding mount via a conduit. For example, the fourth hub 6004D may not be in fluid communication with the mount 6002D.

In some embodiments, the conduit 6006A-C may remain connected to its corresponding hub 6004A-C during manual control. In some embodiments, the conduits 6006A-C may have a sufficient length to advantageously provide flexibility for the physician to manually control the hubs 6004A-C away from the corresponding mounts 6002A-C. In some embodiments, monitoring and mitigating bubbles and/or air pockets within the fluidics system may be difficult with longer conduits 6006A-C. The conduit 6006A-C may be between 6 inches and 24 inches in length. For example, the conduit 6006 can be 6 inches, 7 inches, 8 inches, 9 inches, 10 inches, 11 inches, 12 inches, 13 inches, 14 inches, 15 inches, 16 inches, 17 inches, 18 inches, 19 inches, 20 inches, 21 inches, 22 inches, 23 inches, or 24 inches, or any intermediate lengths. In such embodiments, the conduit 6006A-C may be sufficiently long to enable a physician to manually control the one or more hubs 6006A-C away from the corresponding mounts 6006A-C while monitoring and mitigating bubbles.

In some embodiments, one or more of the conduit 6006A-C may be uncoupled from its corresponding hub 6004A-C. In some such embodiments, the hub 6004A-C may be coupled to the fluidics system using an alternative connection (e.g., an alternative conduit or tubing set). In some embodiments, the hub 6004A-C may be fluidly connected to a secondary fluidics source. For example, the hub 6004A-C may be fluidly connected to a saline and/or contrast bag or other fluid source. In some embodiments, the secondary fluidics source can be a gravity fed or pressurized fluidics bag. In some embodiments, the hub 6004A-C can be coupled to a vacuum source, such as a syringe for aspiration. In some embodiments, the fluidics may be connected through a T-connector. The T-connector can be in fluid communication with a saline and/or contrast bag and/or an aspiration source. For example, the T-connector can fluidly connect the one or more detached interventional devices to a syringe.

In some embodiments, one or more hubs 6004A-D may remain coupled to the corresponding mount 6002A-D during an entirety of a procedure. This may advantageously allow the physician to maintain a connection between an interventional device of the hub remaining coupled to its corresponding mount and the fluidics system (e.g., via conduit 6006A) throughout the duration of the procedure. This may be advantageous in procedures in which an interventional device can be robotically driven to a particular position and further movement of the interventional device is not desired throughout remaining portions of the procedure. For example, in some embodiments the hub 6004A coupled to the guide catheter 2906 can be driven to achieve supra-aortic access. Further distal movement of the guide catheter may not be required in the procedure. In such embodiments, the hub 6004A may remain robotically attached to the mount 6002A for the duration of the procedure. This may advantageously allow the physician to maintain a connection between the guide catheter 2906 and the fluidics system (e.g., via conduit 6006A) throughout the duration of the procedure.

As shown in FIGS. 30A-30B, in certain embodiments, an interventional device assembly may include a plurality of hub assemblies. In some embodiments, the plurality of hub assemblies may include a first hub assembly 6000A, a second hub assembly 6000B, a third hub assembly 6000C, and a fourth hub assembly 6000D. Each of the plurality of hub assemblies 6000A-D can include a corresponding first subassembly or hub 6004A-D, a corresponding second subassembly or mount 6002A-D, and one or more anti-buckling devices 6181A, 6181B, 6181C, and 6181D, which may be in the form of telescoping tubes. The one or more anti-buckling devices 6181A, 6181B, 6181C, and 6181D may provide support to one or more interventional devices extending between the one or more hub assemblies.

In some embodiments, a first anti-buckling device 6181A can extend from the first hub 6004A to a distal support at a distal portion of the drive table having a support surface. The first anti-buckling device 6181A may be removably coupled to the distal support to allow for uncoupling of the first anti-buckling device 6181A before uncoupling the first hub 6004A from the first mount 6002A.

A second anti-buckling device 6181B can extend between the second hub 6004B and the first hub 6004A and can be removably coupled to the first hub 6004A. In some embodiments, if uncoupling of the first hub 6004A from the first mount 6002A is desired, but not uncoupling of the second hub 6004B from the second mount 6002B, the second anti-buckling device 6181B may be uncoupled from the first hub 6004A prior to uncoupling the first hub 6004A from the first mount 6002A. In other embodiments, if both the first hub 6004A and the second hub 6004B are uncoupled from their respective mounts, the second anti-buckling device 6181B may remain coupled to the first hub 6004A. In some embodiments, the second anti-buckling device 6181B may be uncoupled from the first hub 6004A before uncoupling the second hub 6004B from the second mount 6002B if the first hub 6004A is not also being uncoupled from the first mount 6002A (as shown, for example, in FIG. 30B). FIG. 30B illustrates the second anti-buckling device 6181B retracted and/or disengaged from the second hub 6004B. The second hub 6004B can be removed from the second mount 6002B while the procedure catheter 2904 is positioned within the guide catheter 2906.

A third anti-buckling device 6181C can extend between the third hub 6004C and the second hub 6004B and can be removably coupled to the second hub 6004B. In some embodiments, if uncoupling of the second hub 6004B from the second mount 6002B is desired, but not uncoupling of the third hub 6004C from the third mount 6002C, the third anti-buckling device 6181C may be uncoupled from the second hub 6004B prior to uncoupling the second hub 6004B from the second mount 6002B. In other embodiments, if both the second hub 6004B and the third hub 6004C are uncoupled from their respective mounts, the third anti-buckling device 6181C may remain coupled to the second hub 6004B (as shown, for example, in FIG. 30B). In some embodiments, the third anti-buckling device 6181C may be uncoupled from the second hub 6004B before uncoupling the third hub 6004C from the third mount 6002C if the second hub 6004B is not also being uncoupled from the second mount 6002B.

A fourth anti-buckling device 6181D can extend between the fourth hub 6004D and the third hub 6004C and can be removably coupled to the third hub 6004C. In some embodiments, if uncoupling of the third hub 6004C from the third mount 6002C is desired, but not uncoupling of the fourth hub 6004D from the fourth mount 6002D, the fourth anti-buckling device 6181D may be uncoupled from the third hub 6004C prior to uncoupling the third hub 6004C from the third mount 6002C. In other embodiments, if both the third hub 6004C and the fourth hub 6004C are uncoupled from their respective mounts, the fourth anti-buckling device 6181D may remain coupled to the third hub 6004B (as shown, for example, in FIG. 30B). In some embodiments, the anti-buckling device 6181D may be uncoupled from the third hub 6004C before uncoupling the fourth hub 6004D from the fourth mount 6002D if the third hub 6004C is not also being uncoupled from the third mount 6002C.

In certain embodiments, as shown in FIGS. 30A-30B, the hubs 6004A-D may be removably secured to the mounts 6002A-D using clamp mechanisms 6007A-D. The clamp mechanisms 6007A-D may be mounted to a respective mount 6002A-D. The clamp mechanisms 6007A-D may include a lever arm. In some embodiments, the lever arm may be configured to rotate about a pivot point. The lever arm can be biased toward the hub 6004A-D. In the biased state, the clamp mechanism 6007A-D may secure a corresponding hub 6004A-D in a mounted position. The clamp mechanism 6007A-D may transition to an unbiased position by depressing the lever arm on a side of a pivot point opposite the hub 6004A-D.

Additional details regarding anti-buckling systems and devices, which may be used with the embodiments described herein, are included in U.S. patent application Ser. No. 18/524,973, entitled Interventional Device Assembly With Anti-Buckling System, filed Nov. 30, 2023, which is hereby expressly incorporated by reference in its entirety herein.

Additional details regarding systems and methods for performing robotic and manual vascular procedures, which may be used in combination or alternatively to the systems and methods described herein, are included U.S. patent application Ser. No. 18/545,687, entitled System With Removable Hubs For Manual And Robotic Procedure, which is hereby expressly incorporated by reference in its entirety herein.

Additional details regarding drive tables, which may be used with the systems and methods described herein, are included in U.S. patent application Ser. No. 18/525,729, entitled Telescoping Drive Table, filed Nov. 30, 2023, U.S. Provisional Patent Application Ser. No. 63/656,547, entitled Drive Table, filed Jun. 5, 2024, and U.S. Provisional Patent Application Ser. No. 63/727,544, entitled Magnetic Coupling Through a Sterile Field Barrier, filed Dec. 3, 2024, each of which is hereby expressly incorporated by reference in its entirety herein.

Additional details regarding control systems, which may be used with the systems and methods described herein, are included in U.S. patent application Ser. No. 18/784,630, entitled System For Remote Medical Procedure, filed Jul. 25, 2024, each of which is hereby expressly incorporated by reference in its entirety herein

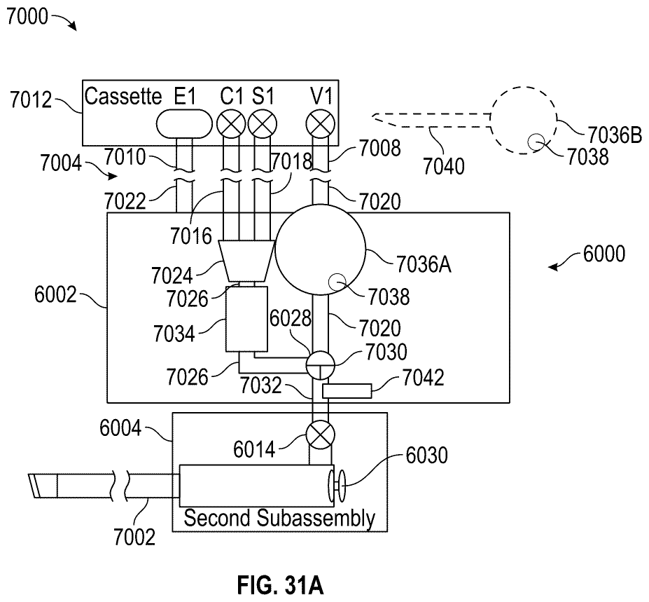

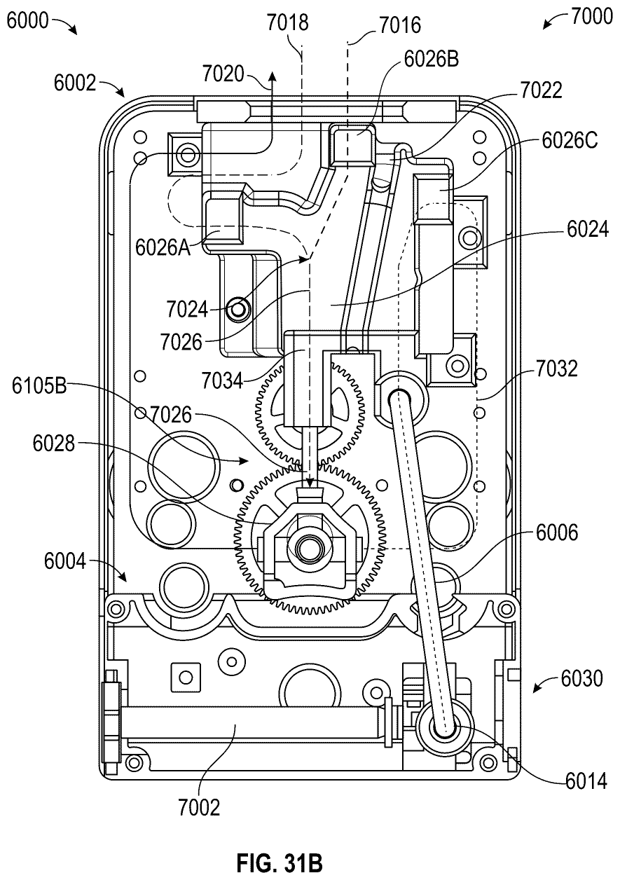

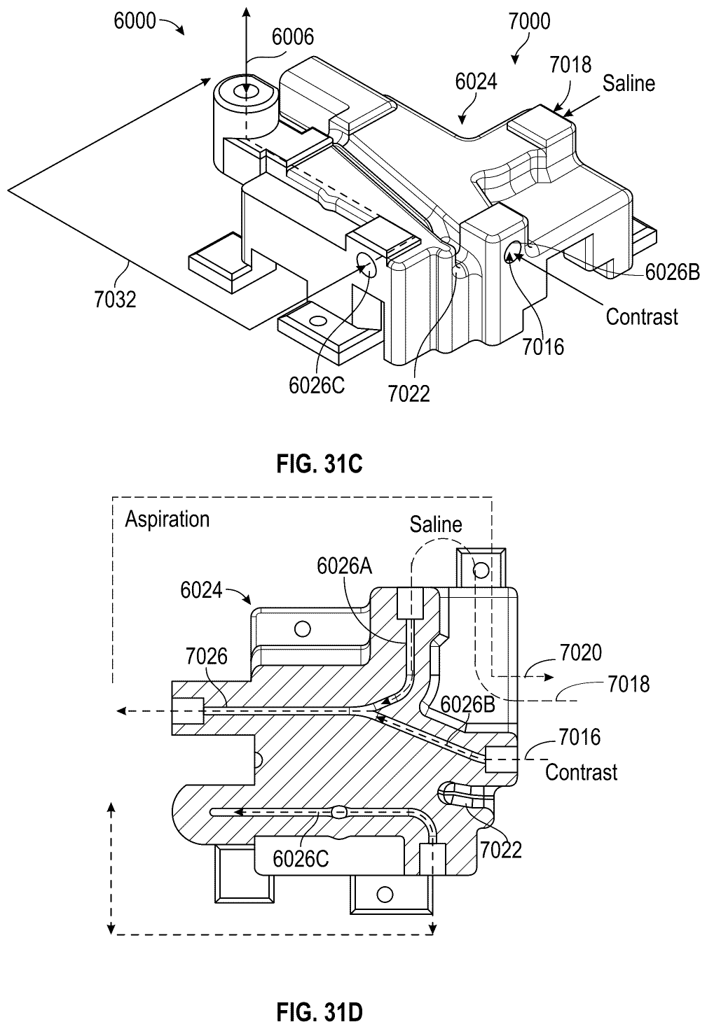

An example of fluidic components in an embodiment of a hub is illustrated in FIGS. 31A-31D.

FIG. 31A illustrates an example of a catheter 7002 (also referred to as an interventional device) coupled to an embodiment of a tubing set 7004 in a hub assembly 6000 fluidics management system. The distal end 7008 of the tubing set 7004 is coupled to the hub assembly 6000 described above and to the catheter 7002, and the proximal end 7010 of the tubing set 7004 can be connected to a cassette 7012, which can include all or part of a saline subsystem, all or part of a contrast subsystem, and/or all or part of a vacuum subsystem of the fluidics management system. Certain components illustrated in FIG. 31A may be described as being part of the tubing set 7004, even though they may be in the hub assembly 6000 or in a portion of a sterile adapter, because they operate to perform part of the fluid and vacuum communication functionality facilitated by the configuration of the tubing set. Means for providing fluid and electrical connections to the catheter can include one or more of the saline subsystem, the contrast subsystem, the vacuum subsystem, and/or electrical connections that are described herein.

As illustrated in FIG. 31A, the catheter 7002 can be coupled to a portion of the tubing set 7004 of the fluidics management system 7000 via the luer 6014. In this example, the portion of the tubing set 7004 includes a contrast tube 7016 connected to port/connection point C1, a saline tube 7018 S1 connected to port/connection point, and an aspiration tube 7020 connected to port/connection point V1. One or more electrical channels 7022 are connected to port/connection point E1 which can be on the cassette 7012 or on the pump station. In some embodiments, the electrical channels 7022 are part of the tubing set 7004, such that the tubing set 7004 includes tubes for communicating saline, contrast, and vacuum tubes from the cassette to the hubs, and electrical connections to the hubs, and such embodiments may be advantageous for wire/tube management. The tubing set 7004 can include a branch point in the form of a two-to-one wye connector 7024 that couples upstream of the contrast tube 7016 and the saline tube 7018 of the tubing set 7004 to a single downstream saline/contrast tube 7026. The tubing set 7004 can further include a three-way valve 6028 that can be actuated by a three-way valve actuator 7030 (for example the gear train 6105B) to selectively place the catheter 7002 in communication with the single downstream saline/contrast tube 7026 or the aspiration tube 7020. In some embodiments, the tubing set 7004 can further include a catheter coupling tube 7032 downstream of the three-way valve 6028 to couple the three-way valve 6028 with the catheter 7002. The three-way valve actuator 7030 can be actuated by a controller. In some embodiments, the three-way valve actuator 7030 includes a drive assembly configured to move the three-way valve 6028. In some embodiments, the three-way valve actuator 7030 includes electromechanical means for moving the three-way valve 6028, the electromechanical means controlled by a controller. In some embodiments, the three-way valve actuator 7030 includes a motor controlled by a controller. In some embodiments, a check valve may be in fluid communication with the saline tube 7016. The check valve can be positioned between a saline source and the two-to-one wye connector 7024. The check valve may advantageously prevent back pressure from the contrast into the saline line.

In some embodiments, the three-way valve 6028 can be a three-way stopcock. The three-way valve 6028 may be actuated (e.g., rotated) to selectively provide or prevent fluid communication between ports coupled to the saline/contrast tube 7026, the aspiration tube 7020, and the catheter coupling tube 7032. The three-way valve 6028 can be actuated to a first position to open a fluid communication channel between the aspiration tube 7020 and the catheter coupling tube 7032, a second position to open a fluid communication channel between the saline/contrast tube 7026 and the aspiration tube 7020, and a third position to open a fluid communication channel between the saline/contrast tube 7026 and the catheter coupling tube 7032. In some embodiments, the three-way valve 6028 can be actuated to a fourth position in which none of the aspiration tube 7020, the saline/contrast tube 7026, and the catheter coupling tube 7032 are in fluid communication.

While a three-way valve 6028 is shown in FIG. 31A, other valve arrangements for selectively placing the catheter 7002 in communication with the saline/contrast tube 7026 or the aspiration tube 7020 may be used.

As shown in FIG. 31A, an air bubble filter 7034 may be positioned between the wye connector 7024 and the three-way valve 6028. In some embodiments, a clot pod 7036 may be positioned along the aspiration tube 7020 upstream of the three-way valve 6028. A clot pod sensor 7038 (in communication to a controller) can be positioned to detect material on the clot pod 7036. In some embodiments, a clot pod 7036 may be positioned in the hub assembly 6000 along the aspiration tube 7020 upstream of the three-way valve 6028. In some implementations, the wye connector 7024, three-way valve 6028, three-way valve actuator 7030, clot pod 7036A, and/or portions of the tubing set 7004 can be housed within a magnetic sterile adapter that may couple with the hub assembly 6000 (also referred to as a puck) and can be considered to be part of a hub assembly. In some embodiments, a clot pod 7036B can be positioned. A clot pod 7036A may be positioned along the aspiration tube 7020 closer to a vacuum canister, for example, between a vacuum manifold 7040 and the vacuum canister.

A hemodynamic pressure sensor 7042 may be positioned between the three-way valve 6028 and the catheter 7002, for example, on the catheter coupling tube 7032. The hemodynamic pressure sensor 7042 is configured to sense a hemodynamic pressure of a patient in which the catheter 7002 is inserted, and provide information relating to the sensed pressure to a controller.

FIGS. 31B-31D illustrate various views of the fluidics management system shown in FIG. 31A implemented within the hub assembly 6000.

FIG. 31B illustrates a top view of the fluidics components within the hub assembly 6000.

As illustrated in FIG. 31B, the catheter 7002 can be coupled in fluid communication with a portion of the fluidics management system 7000 via the luer 6014. The luer 6014 can be coupled to the hemostasis valve to provide fluid communication between the hemostasis valve and the fluidics management system 7000. As described above, the fluidics management system 7000 can include tubes for communicating saline, contrast, and vacuum from the cassette to the hub assembly 6000, and electrical connections to the hub assembly 6000, and such embodiments may be advantageous for wire/tube management. In this example, the portion of the fluidics management system 7000 includes a contrast tube 7016 connected to port/connection point, a saline tube 7018 connected to port/connection point, and an aspiration tube 7020 connected to port/connection point. The contrast tube 7016 and the saline tube 7018 provide contrast and saline to the fluidics management system, respectively, as illustrated by the arrows.

The manifold 6024 can include a branch point in the form of a two-to-one wye connector 7024 that couples upstream of the contrast tube 7016 and the saline tube 7018 of the tubing set 7004 to a single downstream saline/contrast tube 7026. The fluidics management system 7000 can further include a three-way valve 6028 that can be actuated by a three-way valve actuator, such as the gear train 6105B to selectively place the catheter 7002 in communication with the single downstream saline/contrast tube 7026 or the aspiration tube 7020. In some embodiments, the fluidics management system 7000 can further include a catheter coupling tube 7032 downstream of the three-way valve 6028 to couple the three-way valve 6028 with the catheter 7002. The gear train 6105B can be actuated by a controller. In some embodiments, the gear train 6105B includes a drive assembly configured to move the three-way valve 6028. In some embodiments, the gear train 6105B includes means for moving the three-way valve 6028. The means can be controlled by a controller. In some embodiments, the gear train 6105B includes a motor controlled by a controller. In some embodiments, the means may be electromechanical, hydraulic, and/or pneumatic means for moving the three-way valve 6028.

In some embodiments, the three-way valve 6028 can be a three-way stopcock as described above. The three-way valve 6028 may be rotationally fixed. The three-way valve 6028 may include a stopcock control 8010. The stopcock control 8010 can be actuated (e.g., rotated) to selectively provide or prevent fluid communication between ports coupled to the saline/contrast tube 7026, the aspiration tube 7020, and the catheter coupling tube 7032. The stopcock control 8010 of the three-way valve 6028 can be actuated to a first position to open a fluid communication channel between the aspiration tube 7020 and the catheter coupling tube 7032; to a second position to open a fluid communication channel between the aspiration tube 7020 and the saline/contrast tube 7026; and a third position to open a fluid communication channel between the saline/contrast tube 7026 and the catheter coupling tube 7032. In some embodiments, the stopcock control 8010 of the three-way valve 6028 can be actuated to a fourth position in which the aspiration tube 7020, the saline/contrast tube 7026, and the catheter coupling tube 7032 are all blocked. In such embodiments, the none of the aspiration tube 7020, the saline/contrast tube 7026, and the catheter coupling tube 7032 are in fluid communication.

FIG. 31C illustrates a perspective view of fluidics management system 7000 implemented within the manifold 6024. The fluidics channels 6026A-6026C are defined by contours and lumens extending through the manifold 6024. In some embodiments, the fluidics channels 6026A, 6026B may converge at a point within the manifold 6024. In some embodiments, the fluidics channel 6026C may extend through a lumen in the manifold 6024. The manifold 6024 may further include a groove or channel 7022 disposed along the top of the manifold 6024. The fluidics channels 6026A, 6026B may be used for providing saline and/or contrast to the three-way valve 6028, respectively. The fluidics channel 6026C may be used to fluidly connect the three-way valve 6028 with an interventional device in the hub 6004. The groove or channel 7022 may be used for guiding electrical wiring across the manifold. The electrical wiring may be used to supply electrical power to a sensor. For example, the electrical wiring may provide electrical power to a pressure sensor.

FIG. 31D illustrates a top cross-sectional view of the fluidics management system 7000 implemented within the manifold 6024. As shown in FIG. 31D, the aspiration line may extend around the manifold 6024. In some embodiments, a check valve may be operatively positioned in fluid communication with the saline tube 7018 prior to the first fluidics channel 6026A. The check valve may advantageously prevent back pressure of the contrast from entering the saline tube.

FIG. 32A illustrates a three-way valve 6028. The three-way valve 6028 can include a body 8002 defining a plurality of ports 8004, 8006, 8008 extending between interior passages and a stopcock control 8010. In some embodiments, the stopcock control 8010 may be controlled by the three-way valve actuator 7030 described above. In some embodiments, the three-way valve may include a first port 8004, a second port 8006, and a third port 8008. One or more lumens may extend between the plurality of ports 8004, 8006, 8008. For example, a first lumen may extend between the second port 8006 and the third port 8008. A second lumen may extend from the first port 8004 to the first lumen. In some embodiments, the second lumen may intersect the first lumen. For example, the second lumen can intersect a midpoint along the longitudinal length of the first lumen. In some embodiments, the first lumen and the second lumen are coplanar. The first lumen can be orthogonal to the second lumen.

The first port 8004 can be in fluid communication with the saline/contrast tube 7026 and configured to provide saline and/or contrast into the three-way valve 6028. The second port 8006 can be in fluid communication with the aspiration tube 7020 and configured to aspirate the three-way valve 6028. The third port 8008 can be in fluid communication with the catheter coupling tube 7032 and configured to connect the saline/contrast tube 7026 and/or aspiration tube 7020 to the catheter 7002 in the hub 6004.

The stopcock control 8010 can be configured to rotate about a central vertical axis. The body 8002 can be coupled to a stopcock control 8010. In some embodiments, rotation of the stopcock control 8010 may change the configuration and flow of the fluidics management system 7000. For example, rotation of the stopcock control 8010 may rotate the first and/or second lumens of the three-way valve 6028. In some embodiments, the gear train 6105B may rotate the stopcock control 8010 relative to the body 8002.

FIGS. 32B-32E illustrate various configurations for the three-way valve 6028. The solid line in the following figures represents a flow path through the three-way valve 6028. The “X” represents a blocked opening thereby preventing the associated tube 7026, 7028, 7032 from being fluidly connected to the other tubes 7026, 7028, 7032. An interior passageway 8011 of the three-way valve can be coupled to the stopcock control 8010 and configured to rotate relative to the body 8002. In some embodiments, the interior passageway 8011 is in a “T” shape wherein two lumens are oriented orthogonally with respect to one another. In some embodiments, a first lumen may extend through the body 8002 and a second lumen may extend from a port of the body 8002 to the first lumen. In some embodiments, a tab portion of the stopcock control 8010 may extend radially from a central portion of the stopcock control 8010. The tab portion may extend in the direction opposite the second lumen. The tab portion of the stopcock control 8010 can extend orthogonally relative to the direction of the first lumen. Accordingly, the direction of the tab portion of the stopcock control 8010 can correspond to a blocked port thereby preventing the corresponding tube from being in fluid communication with the catheter 7002.

FIG. 32B illustrates a first configuration 8012 of the three-way valve 6028. The first configuration 8012 can be configured to provide aspiration to the catheter 7002 by selectively connecting the aspiration tube 7028 with the catheter connection tube 7032 through the interior passageway 8011. The first configuration 8012 illustrates the tab portion of the stopcock control 8010 aligned with the first port 8004. As described above, the first port can be in fluid communication with the contrast/saline tube 7026. As further illustrated, the interior passageway 8011 is shown blocking off the first port 8004. The first configuration 8012 can be useful in aspirating a clot or other bodily fluids from a patient through the catheter 7002.

FIG. 32C illustrates a second configuration 8014 of the three-way valve 6028. The second configuration 8014 can be configured to aspirate saline and/or contrast by selectively connecting the contrast/saline tube 7026 with the aspiration tube 7028 through the interior passageway 8011. The third configuration 8016 illustrates the tab portion of the stopcock control 8010 aligned with the third port 8008. As described above, the third port 8008 can be in fluid communication with the catheter connection tube 7032. As further illustrated, the interior passageway 8011 is shown blocking off the third port 8008. The third configuration 8016 can be useful in cleaning the interior passageway 8011 of the three-way valve 6028 and/or removing air bubbles from the contrast/saline tube 7026.

FIG. 32D illustrates a third configuration 8016 of the three-way valve 6028. The third configuration 8016 can be configured to provide contrast and/or saline to the catheter 7002 by selectively connecting the contrast/saline tube 7026 with the catheter connection tube 7032 through the interior passageway 8011.

The second configuration 8014 illustrates the tab portion of the stopcock control 8010 aligned with the second port 8006. As described above, the second port 8006 can be in fluid communication with the aspiration tube 7028. As further illustrated, the interior passageway 8011 is shown blocking off the second port 8006. The second configuration 8014 can be useful in proving saline or contrast to a patient through the catheter 7002 (e.g., for cleaning, priming, and/or capturing images).

FIG. 32E illustrates a fourth configuration 8018 of the three-way valve 6028. The third configuration 8018 can be configured to turn off the three-way valve 6028 by selectively positioning the interior passageway in a neutral position between the first, second, and third ports 8004, 8006, 8008. The fourth configuration 8018 illustrates the tab portion of the stopcock control 8010 in a neutral position between the first port 8004 and the third port 8008. As further illustrated, the interior passageway 8011 is shown blocking off the first port 8004, the second port 8006, and the third port 8008. The fourth configuration 8018 can be useful to prevent fluids from transferring through the catheter 7002.

Other configurations are also possible. For example, the stopcock control 8010 may be rotated at any angle around the circumference of the output gear of the second gear train 6105B. In some embodiments, the stopcock control 8010 can fluidly connect all three ports of the three-way valve 6028.

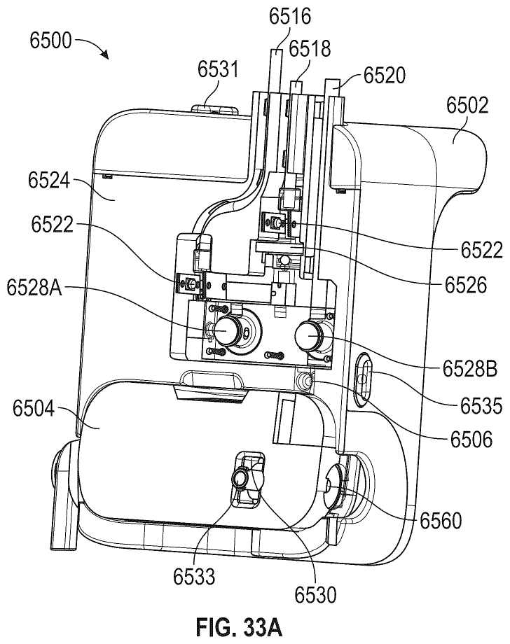

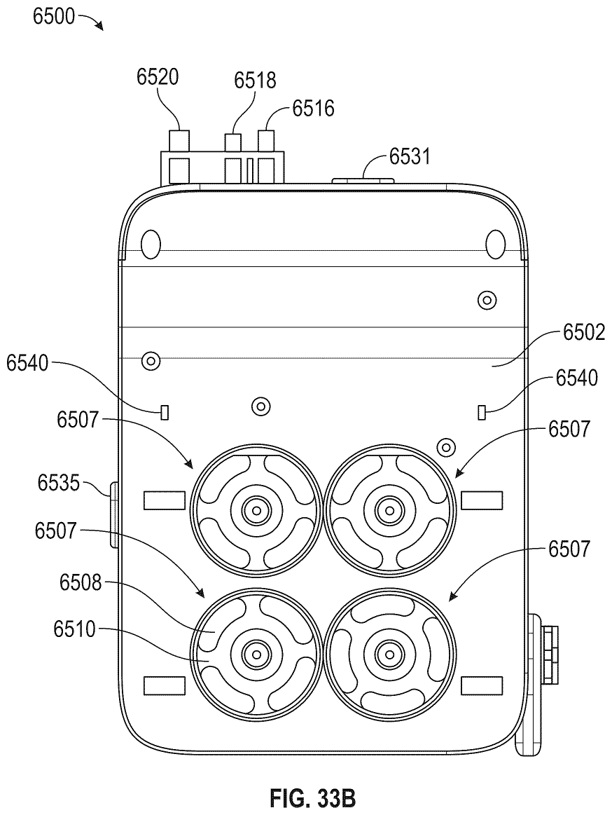

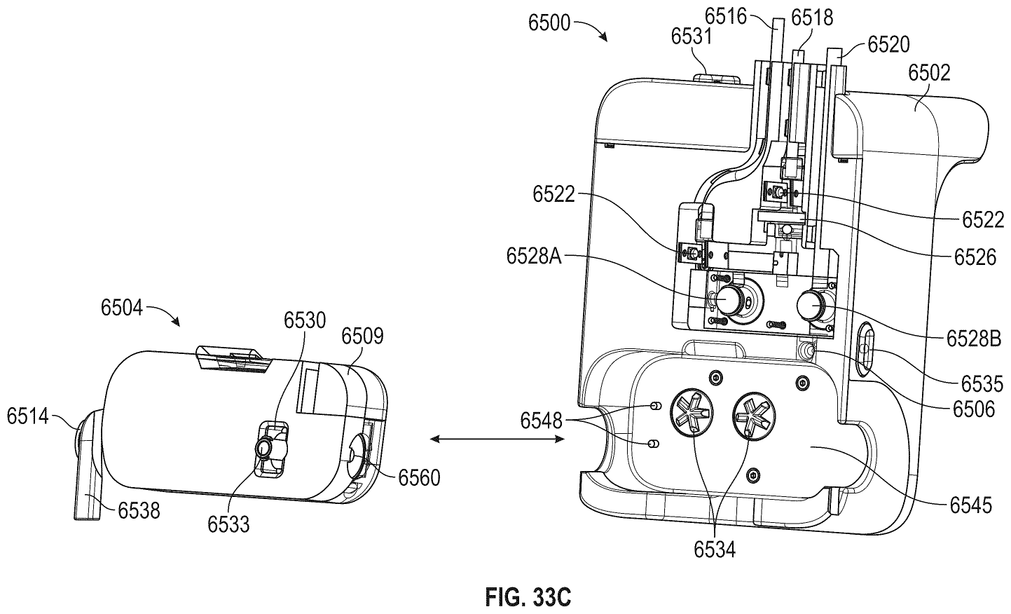

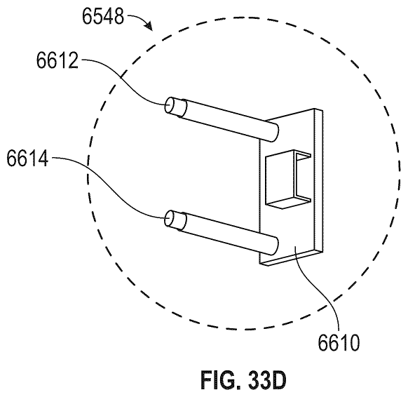

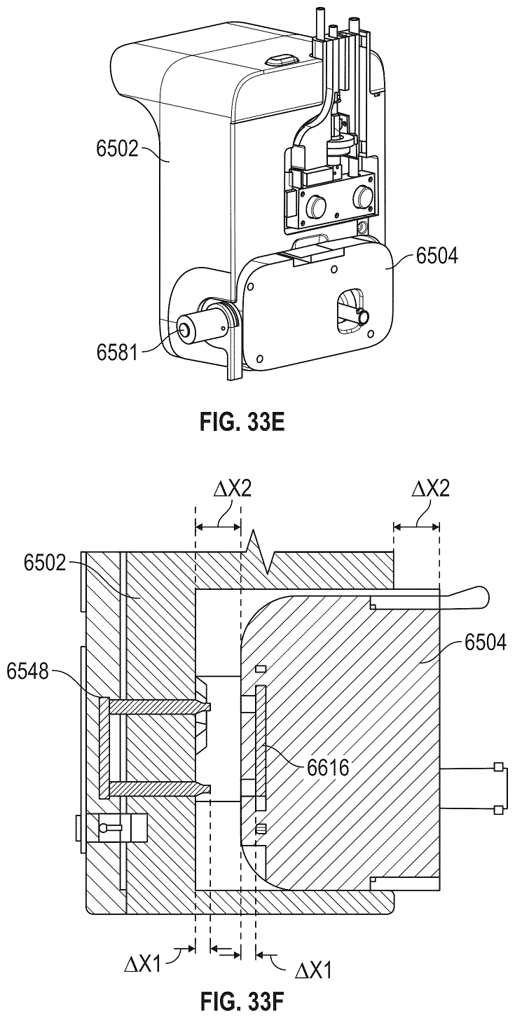

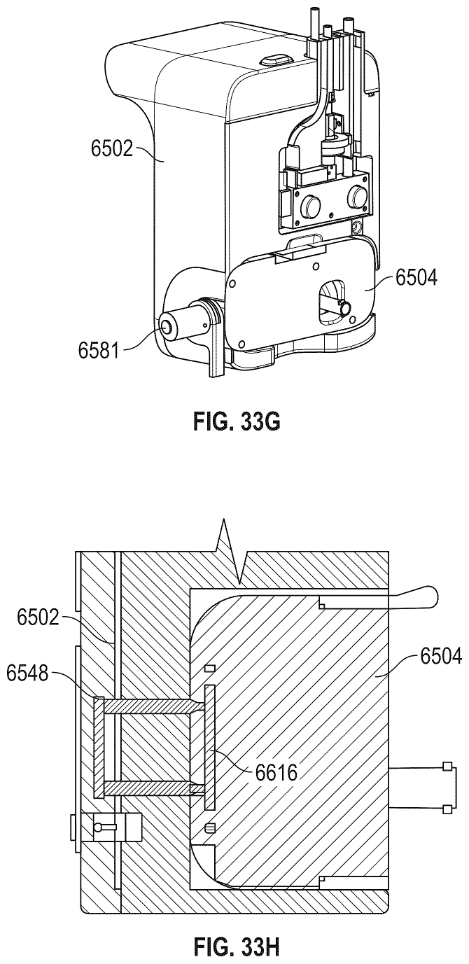

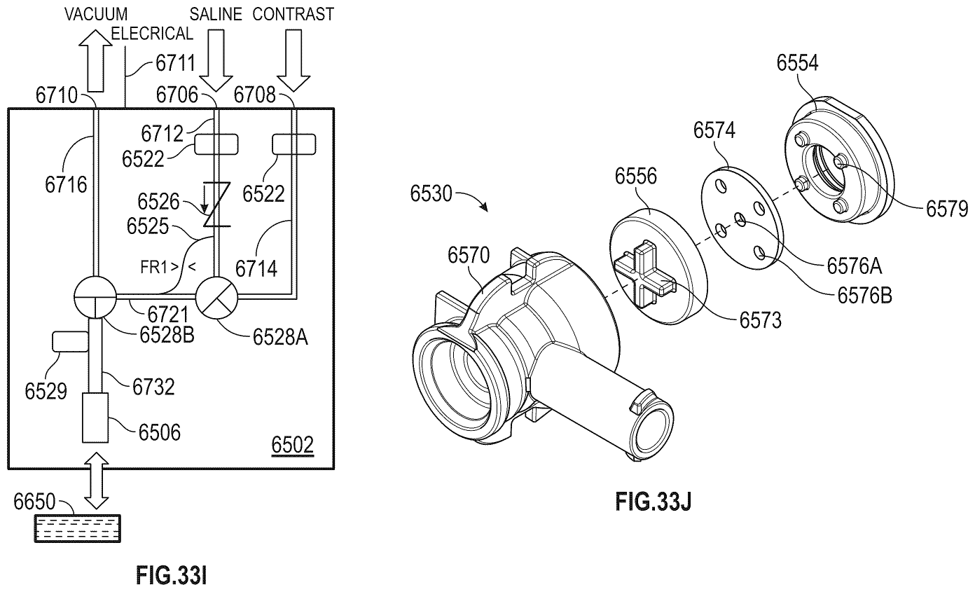

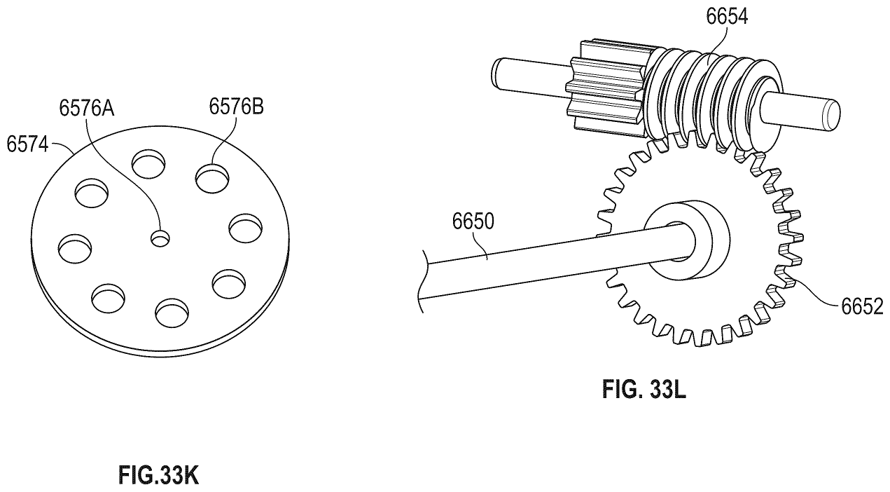

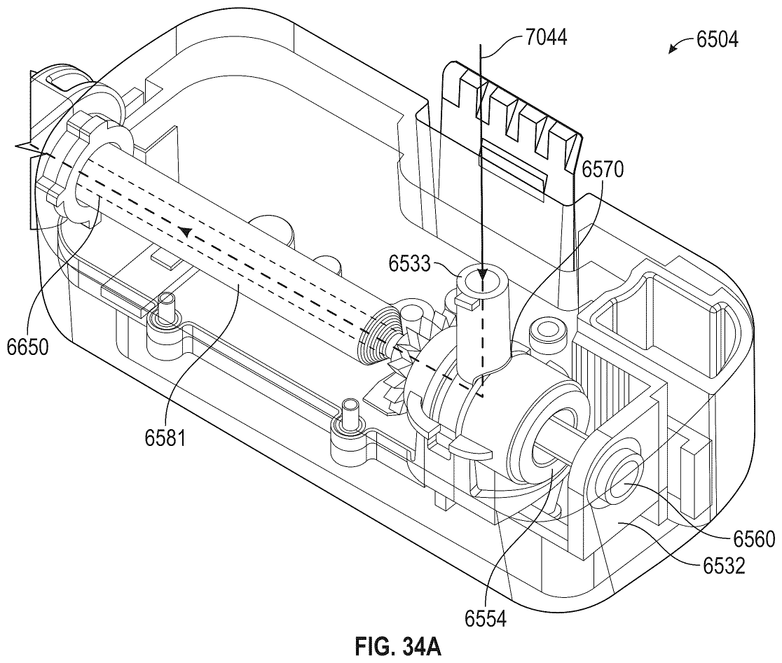

FIGS. 33A-33L depict an embodiment of a hub assembly 6500. The hub assembly 6500 may include any of the same or similar features and/or functions as any of the hubs or hub assemblies described herein (e.g., hub assembly 6000).

In some embodiments, the hub assembly 6500 can include a first subassembly, puck, or mount 6502 and a second subassembly or hub 6504. The mount 6502 can also be referred to as a catheter puck, a hub mount, and/or a first hub member. The mount 6502 can be configured to couple to and move along a drive table. The hub assembly 6500 can be configured to be positioned on a sterile side (e.g., a disposable equipment side) of a sterile barrier. The mount 6502 can include any of the same or similar features and/or functions as any of the mounts described herein (e.g., mount 6002).

In some embodiments, the hub 6504 can be referred to as a second hub member. The hub 6504 may include or couple to an interventional device, such as a catheter or guidewire. The hub 6504 can include any of the same or similar features and/or functions as any of the hubs described herein (e.g., hub 6004).

As described herein, in certain embodiments an interventional device may be coupled to a fluidics management system (e.g., to receive fluids such as contrast or saline, or for aspiration). In some embodiments, the mount 6502 can be coupled to the fluidics management system.

In some embodiments, a contrast tube, a saline tube, and/or an aspiration tube may be coupled to or extend into the mount 6502 (e.g., through one or more openings in a housing thereof) to provide contrast, saline, and/or vacuum to the hub assembly 6500. Additionally, in some embodiments, a power line may couple to or extend into the mount 6502 (e.g., through one or more openings in a housing thereof) to provide electrical power into the mount 6502. For example, the mount 6502 can be coupled to or receive a contrast line or contrast tube 6516. The mount 6502 can be coupled to or receive a saline line or saline tube 6518. The mount 6502 can be coupled to or receive an aspiration line or tube 6520.

In some embodiments, the mount 6502 can include a manifold 6524. The manifold 6524 can include any of the same and/or similar features and functions as the manifold 6024 and vice versa. The manifold 6524 can be configured to receive and redistribute the fluidics within the mount 6502 as described in greater detail below. The manifold 6524 may include one or more fluidic channels. In some embodiments, the manifold 6524 can be configured to receive and/or transport saline and/or contrast to a valve 6528A. The valve 6528A may be a three-way valve. In some embodiments, the manifold 6524 can be configured to receive and/or transport saline and/or contrast to a valve 6528B (e.g., from the valve 6528A). The valve 6528B may be a three-way valve. In some embodiments, the manifold 6524 can be configured to aspirate the valve 6528B to flush the valve 6528B and/or receive bodily fluids from the three-way valve 6528B.

In certain embodiments, the mount 6502 can include one or more air bubble sensors 6522. For example, an air bubble sensor 6522 may be positioned along a saline flow path proximal to the valve 6528A. An air bubble sensor 6522 may be positioned along the contrast flow path proximal to the valve 6528A. In certain embodiments, the mount 6502 can include a check valve 6526. The check valve 6526 may be positioned along a saline flow path.

In some embodiments, a fluidics connector (e.g., as described with respect to hub assembly 6000) can extend between and fluidly couple the mount 6502 and the hub 6504 (e.g., to provide fluid and/or aspiration to the hub 6504). In certain embodiments, the mount 6502 may include a connector or fluid port 6506 distal to the valve 6528B for receiving fluid or aspiration from the valve 6528B. The fluid port 6506 can be placed in fluid communication with a fluidics system for the delivery of fluids (e.g., saline and/or contrast media) to the interventional device and/or for aspiration of fluids from the interventional device. For example, in some embodiments, the fluid port 6506 can be in fluid communication with the three-way valve 6528B. The fluid port 6533 can include a luer connector (e.g., a female luer connector) configured to couple to a luer (e.g., a male luer connector) of a fluidics connector coupling the mount 6502 and the hub 6504.

In certain embodiments the manifold 6524 and/or other fluidics components of the mount may be removably coupled to a housing base of the mount (e.g., for case of assembly).

FIG. 33I is a schematic illustrating an example of a fluidic system components in the mounts 6502. The mount 6502 includes fluidic connections to the tubes 6516, 6518, and 6520 to receive saline, contrast, and vacuum, and an electrical connection that includes one or more electrical leads, which are coupled to one or more sensors in the mount 6502 to communicate signals from one or more sensors in the mount to a controller. A controller can use the received signals to align valves in the mount 6502 individually to provide saline, contrast, and aspiration (vacuum) automatically or semi-automatically (e.g., based on a user input) as needed during different portions of a medical procedure. In this example, mount 6502 has a common connector or fluid port 6506 for providing saline, contrast, and vacuum to the lumen of a catheter 6650 of the hub 6504. While a catheter 6650 is shown in FIG. 33I, one of skill in the art would understand that the hub 6504 and features thereof can be used with other interventional devices (e.g., guidewires) as described herein.

The connector or fluid port 6506 can be in fluid communication with the lumen of catheters 6650 by coupling the connector of fluid port 6506 to a corresponding connector or fluid port 6533 on the hub 6504 that the catheter 6650 is coupled to, where the hub 6504 includes a fluid communication channel from the connector or fluid port 6533 to the lumen of the catheter coupled to the hub 6504.

The hub 6504 can include structure that operates as a hemostasis valve 6530, such that the hub 6504 can provide fluid to the lumen of the catheter 6650 coupled to the hub 6504 for discharging at the distal end of the catheter 6650, or can provide vacuum to the lumen of the catheter 6650 for providing aspiration at the distal end of the catheter 6650 (e.g., in some embodiments, while another interventional device is positioned partially or fully in the lumen of the catheter 6650 in a concentric nested configuration).

The mount 6502 can include a saline port 6706, which may receive or couple to the saline tube 6518, a contrast port 6708, which may receive or couple to the contrast tube 6516, a vacuum port 6710, which may receive or couple to the vacuum tube 6520, and an electrical connection 6711. As described above, an air bubble sensor 6522 is positioned along a saline channel (flow-path) 6712 and is in electrical communication with electrical connection 6711. The air bubble sensor 6522 is configured to detect air in the saline channel 6712 and generate a signal indicative of detecting air which is provided to the controller. An air bubble sensor 6522 is positioned along contrast channel (flow-path) 6714 and is in electrical communication with electrical connection 6711. The air bubble sensor 6522 is configured to detect air in the contrast channel 6714 and generate a signal indicative of detecting air which is provided to the controller.

The valve 6528A can be controllable by a controller to align saline channel 6712, or contrast channel 6714, to the valve 6528B. The check valve 6526 in the saline channel 6712 can prevent any upstream fluid flow on the saline channel 6712. Along the saline channel 6712 between the valve 6528A and the check valve 6526 can be a saline drip channel (saline restricted-flow channel) 6525 which connects the saline channel 6712 upstream of the valve 6528A to a fluid flow-path to the valve 6528B, bypassing the valve 6528A. In this configuration, at least some saline that enters the mount 6502 can flow to via the saline restricted-flow channel 6525 to the valve 6528B regardless of the position of the valve 6528A. The saline drip flow-path can include a saline restricted-flow channel 6525 designed to allow a desired flow-rate of saline (e.g., mL/minute) to flow to the connector or fluid port 6506 and to the lumen of a catheter in fluid communication with the connector or fluid port 6506 when the valve 6528B is aligned to provide saline or contrast to the connector or fluid port 6506 regardless of the alignment of the valve 6528A. The saline restricted-flow channel 6525 is configured to provide a lower flow-rate of saline than the saline channel 6712 provides to the saline-contrast channel 6721 through the valve 6528A. The saline restricted-flow channel 6525 can have, for example, a smaller cross-sectional dimension then the cross-sectional dimension of the flow-path from the saline channel 6712 to the saline-contrast channel 6721 through the valve 6528A. The saline restricted-flow channel 6525 can have a flow restrictor (value, size) FR 1 designed such that the saline restricted-flow channel 6525 provides about 1 mL/minute of saline to the lumen of the catheter.

The valve 6528B can be a three-way valve that connects either vacuum to or saline/contrast to connector or fluid port 6506 via a fluid primary channel 6732. The valve 6528B can be controllable by a controller to align a vacuum channel 6716 or the saline-contrast channel 6721 to the primary fluid channel 6732. The primary channel 6732 can provide saline, contrast and vacuum to a catheter coupled to the mount 6502, for example, provides saline, contrast and vacuum to the lumen of the catheter 6650 through connector or fluid port 6506. The primary channel can also receive material (e.g., fluids, clots, etc.) from a catheter coupled to the mount 6502 when vacuum is provided to the catheter. Pressure sensor 6529 (e.g., a hemodynamic pressure sensor) can be positioned to detect pressure in the primary channel 6732 and is in electrical communication with electrical connection 6711. Pressure sensor 6529 can be configured to generate a signal, indicative of the detected pressure, which is provided to the controller.

As shown in FIG. 33B, the mount 6502 can include a passive torque subsystem as described herein. In some embodiments, the mount 6502 can include one or more passive torque elements 6507. Each of the passive torque elements 6507 can be actuated via a magnetic coupling with an active torque element as described herein. As shown in FIG. 33B, in certain embodiments, the mount 6502 may include four passive torque elements 6507. The passive torque elements can be coupled (e.g., via one or more gear trains, or other coupling mechanisms) to one or more features of the hub assembly to actuate the features of the hub assembly.

In some embodiments, each passive torque element 6507 may include one or more magnets configure to magnetically couple with corresponding magnets of an active torque element of a hub adapter. In certain embodiments, a passive torque element 6507 may include a monolithic magnet. In certain embodiments, the passive torque element 6507 can include a polymagnet. The passive torque element 6507 can include one or more first magnetic regions 6508 and one more second magnetic regions 6510 having a second polarity. In some embodiments, the one or more first magnetic regions 6508 can have an opposite polarity than the one or more second magnetic regions 6510. The one or more first magnetic regions 6508 and one or more second magnetic regions 6510 may be part of a single magnet (e.g., a polymagnet). In other embodiments, the one or more first magnetic regions 6508 can include one or more separate magnets, and the one or more second magnetic regions 6510 can include one or more separate magnets.

In certain embodiments, one of the passive torque elements 6507 can be coupled to an interventional device (e.g., catheter or guidewire) coupled to the hub 6504 so that actuation of the passive torque element 6507 (e.g., rotation of the passive torque element 6507) causes rotation of the interventional device as described herein. In certain embodiments, one of the passive torque elements 6507 can be coupled to a hemostasis valve 6530 of the hub 6504 so that actuation of the passive torque element 6507 actuates the hemostasis valve 6530 between various states, as described herein. In certain embodiments, one of the passive torque elements 6507 can be coupled to the valve 6528A so that actuation of the passive torque element 6507 causes the valve 6528A to change fluidics configurations (e.g., rotate) as described herein. In certain embodiments, one of the passive torque elements 6507 can be coupled to the valve 6528B so that actuation of the passive torque element 6507 causes the valve 6528B to change fluidics configurations (e.g., rotate) as described herein.

In certain embodiments, the mount 6502 can include one or more sensors 6540. The sensors 6540 may be configured to detect one or more detectable objects, for example, of a hub adapter positioned across a sterile barrier. The sensors 6540 may be magnetic sensors (e.g., hall sensors). In certain embodiments, each hub adapter may include a unique set of detectable objects (e.g., magnets have particular polarities) that may be detected by the sensors 6540 to identify the hub adapter. In some embodiments, the sensors 6540 may also be positioned to detect alignment of the mount 6502 with a hub adapter.

The mount 6502 can include one or more output members 6534 (e.g., output gears). The output members 6534 can provide an output of the mount 6502 as an input of the hub 6504. For example, the hub 6504 may include one or more input members (e.g., input gears) that can couple with the output members 6534 of the mount 6502.

The hub 6504 may be removably coupled to the mount 6002. As shown in FIG. 33C, the mount 6502 can include a chamber or hub receptacle 6545. The hub receptacle 6545 can be configured to receive the hub 6504. In some embodiments, the receptacle can be shaped, dimensioned, and/or otherwise configured to align the output members 6534 of the mount 6502 with input members of the hub 6504 when the hub 6504 is received therein.

The hub receptacle 6545 can be shaped, dimensioned, and/or otherwise configured to secure (e.g., removably secure) the hub 6504 within the receptacle when the hub 6504 is received therein.

The hub 6504 can include the hemostasis valve 6530 as described herein. The hemostasis valve can include a connector or fluid port 6533 that can be placed in fluid communication with a fluidics system for the delivery of fluids (e.g., saline and/or contrast media) to the interventional device and/or for aspiration of fluids from the interventional device. For example, in some embodiments, the fluid port 6533 can be in fluid communication with the three-way valve 6528B. The fluid port 6533 can include a luer connector (e.g., a female luer connector) configured to couple to a luer (e.g., a male luer connector) of a fluidics connector coupling the mount 6502 and the hub 6504. The luer connector may connect the fluid port 6506 of the mount 6502 with the fluid port 6533 of the hub 6504.

As described herein, the hemostasis valve 6530 can be configured to receive a more proximal interventional device therethrough. For example, the hemostasis valve 6030 can include the channel 6060 for receiving a more proximal interventional device therethrough. In some embodiments the channel 6060 may be coaxial with the interventional device lumen 6015.

In some embodiments, the hub 6504 can include an actuator 6509. The actuator 6509 may be a button, slider, or tab. The actuator 6509 can be manually actuated to manually actuate the hemostasis valve 6530. This may be useful, for example, when the hub 6504 is uncoupled from the mount 6502 and used manually in a procedure. The actuator 6509 can be coupled to a plunger of the hemostasis valve 6530.

In certain embodiments, the mount 6502 may include an actuator 6535. The actuator 6535 may be a button, slider, or tab. The actuator 6535 can be manually actuated by a user to actuate the hemostasis valve 6530 when the hub 6504 is coupled to the mount 6502. For example, in some embodiments, actuation of the actuator 6535 may cause actuation of the actuator 6509 to actuate the hemostasis valve 6530. In other embodiments, actuation of the actuator 6535 may actuate the gear train that causes actuating of the hemostasis valve 6530.

In some embodiments, the mount 6502 may include an actuator 6531. The actuator 6531 may be a button, slider, or tab. The actuator 6531 may be manually actuated by a user to flush the fluidics management system. For example, actuating the actuator 6531 can transmit an electrical signal to actuate one or more valves within the mount 6502 for controlling fluid flow through the fluidics management system.

The hub 6504 can be configured to receive one or more inputs from the mount 6502. The hub 6504 can be configured to transmit one or more outputs. In some embodiments, the hub 6504 may transform the outputs of the mount 6502 into corresponding linear and/or rotary motion of components within or coupled to the hub 6504 (e.g., the interventional device coupled to the hub 6504 and/or one or more fluidics components).

As described herein, the hub 6504 can include an input member (e.g., a gear or socket) in communication with an interventional device (e.g., catheter or guidewire) of the hub 6504 (e.g., via a gear train) to cause rotation of the interventional device in response to receiving an input from the mount 6502 (e.g., from one of the output members 6534). The hub 6504 can include an input member in communication with the hemostasis valve 6530 of the hub 6504 (e.g., via a gear train) to actuate the hemostasis valve between various states in response to receiving an input from the mount 6502 (e.g., from one of the output members 6534). The hemostasis valve 6530 can include a channel 6560 for receiving a more proximal interventional device therethrough, as described herein.

In certain embodiments, a hub 6504 may not include a hemostasis valve. For example, a guidewire hub may not include a hemostasis valve. In such embodiments, the hub 6504 may couple to the mount 6502 without the output members 6534 configured to actuate the hemostasis valve causing a corresponding action in the hub. For example, the hub 6504 may include a corresponding rotating member configured to couple with the output member 6534 but not configured to actuate any other features of the hub 6504. In some embodiments, rotation of an interventional device of a hub 6504 may not be desirable. In such embodiments, the hub 6504 may couple to the mount 6502 without the output members 6534 configured to cause rotation of the interventional device causing a corresponding action in the hub. For example, the hub 6504 may include a corresponding rotating member configured to couple with the output members 6534 but not configured to actuate any other features of the hub 6504. In this way, a standard mount may be used to couple to different types of hubs 6504 (e.g., hubs having different interventional devices). This may allow for replacing a hub 6504 of a first type with a hub 6504 of a second different type within the same mount 6502.

The hub 6504 can include an anti-buckling device assembly or anti-buckling device 6581. The anti-buckling device 6581 can include a telescoping tube, as show, for example, in FIGS. 30A-30B. A telescoping tube can include a proximal retainer that may secure a proximal end of the anti-buckling device 6581 to the hub 6504 (e.g., within the hub 6504). The telescoping tube can include a distal retainer 6514 that can be extended away from the hub 6504 and coupled to a more distal hub or a distal attachment along a drive table. The telescoping tube can extend between the proximal retainer and the distal retainer 6514. The telescoping tube can include a plurality of concentric axially extendable and collapsible tube segments. In some embodiments, an outermost tube segment may be attached to the distal retainer 6514 and an inner most tube segment may be attached to the proximal retainer. In some embodiments, the proximal retainer is attached to a proximal portion of the interventional device within the hub 6504.

The distal retainer 6514 can include an engagement tab 6538. The engagement tab 6538 may be an arm. The engagement tab 6538 can be configured to be manipulated by a user to rotate the distal retainer 6514. The engagement tab 6538 also be configured to be manipulated by a user to extend/collapse the telescoping tube of the anti-buckling device 6581. The engagement tab 6538 can be configured to be manipulated by a user to seat/unseat the distal retainer 6514 within an attachment portion of a more distal hub or distally along the drive table. The engagement tab 6538 can also be configured to seat/unseat the distal retainer 6514 within an attachment of the hub 6504 (e.g., for storage before the anti-buckling device 6581 is extended and coupled to a more distal hub).

In certain embodiments, the mount can include one or more electrical connectors configured to couple with the hub and establish electrical and/or data communication therewith. As shown in FIGS. 33C and 33D, in certain embodiments, the mount 6502 can include a pin assembly 6548. The pin assembly 6548 can be positioned within a hub receptacle 6545 of the mount 6502. For example, the pin assembly 6548 can be positioned within a portion of the mount 6502 configured to receive a corresponding hub 6504. The pin assembly can receive electricity, for example, through the electrical connection 6711. The pin assembly 6548 can be configured to contact electrical contacts within the hub 6504. The pin assembly 6548 may contact the electrical contacts within the hub 6504 to receive information regarding the hub (e.g., bibliographic information, such as a model number, type of hub, or type of interventional device coupled to the hub). Contact with the electrical contacts can also be used to determine presence of the hub 6504 within the mount 6502. The pin assembly 6548 can contact the electrical contacts of the hub 6504 to establish electrical and data communication between the mount 6502 and the hub 6504.

In some embodiments, the pin assembly 6548 can be configured to complete an electrical circuit when a hub 6504 is fully inserted within the mount 6502. The pins of the pin assembly 6548 can be electrical connector pins. For example, the pins of the pin assembly 6548 may contact electrical contacts within the hub 6504 when the hub is 6504 is fully secured to the mount. Accordingly, the pin assembly 6548 can be configured to detect whether a hub 6504 is fully secured to the mount 6502 if the electrical circuit is completed. Full securement of the hub 6504 can mean that the hub 6504 is removably locked in position within the hub 6504.

As shown in FIG. 33D, the pin assembly 6548 can include a circuit board portion 6610, a first pin 6612, and a second pin 6614. The first pin 6612 and the second pin 6614 can extend away from the circuit board portion 6610. As further shown in FIG. 33D, the first pin 6612 and the second pin 6614 can extend externally from the mount 6502. Although two pins 6612 and 6614 are shown, other embodiments may include one pin or more than two pins. In some embodiments, the pins 6612 and 6614 can be pogo pins or spring-loaded pins.

FIGS. 33E-33F respectively illustrate a perspective view and a side view of a state in which a hub 6504 is not fully inserted within the mount 6502. As shown in FIG. 33F, the first pin 6612 and the second pin 6614 may extend from a first surface of the mount 6502 by a first distance ΔX1. The first distance ΔX1 can also correspond to the distance between a circuit board portion 6616 and a first surface of the hub 6504. Accordingly, the first pin 6612 and the second pin 6614 may extend form the first surface of the mount 6502 by a distance sufficient to contact the circuit board portion 6616 when the hub 6504 is fully secured to the mount 6502. The distance may not be sufficient to contact the circuit board portion 6616 when the hub 6504 is not fully secured to the mount 6502. As shown in FIG. 33F, the distance ΔX2 correspond to the distance between the first surface of the mount 6502 and the first surface of the hub 6504. The distance ΔX2 can also correspond the distance the hub 6504 must travel to be fully secured to the mount 6502.

FIGS. 33G-33H respectively illustrate a perspective view and a side view of a state in which a hub 6504 is fully inserted within the mount 6502. In such cases, the distance ΔX2 can be negligible. As shown in FIG. 33H, the first pin 6612 and the second pin 6614 may contact the circuit board portion 6616 (e.g., may contact electrical contacts of the circuit board portion 6616). Accordingly, a circuit can be completed between the first pin 6612 and the second pin 6614. Completing the circuit may indicate that the hub 6504 is fully secured to the mount 6502. Additionally, the circuit board portion 6616 can include or receive information (e.g., bibliographic information) of the hub 6504. In some cases, the circuit board portion 6616 can include one or more identifiers. For example, the circuit board portion 6616 can include a part number, a hub type, model number, etc. The mount 6502 can be configured to identify the hub 6504 has been coupled to the mount 6502.

FIG. 33J depicts an exploded view of the hemostasis valve 6530. In some embodiments, the hemostasis valve can include a body 6570. The hemostasis valve 6530 can include a gasket or seal 6556. The seal 6556 can include a proximal membrane on its proximal end and a distal membrane 6573 on its distal end. The distal membrane 6573 can include a thickened sidewall having a slit therethrough that can provide additional seal contact around an interventional device extending through the seal 6556.

Under high pressure, the seal 6556 may provide a radial seal around an interventional device extending therethrough. However, under low pressure, the seal 6556 may be subject to leak. In some embodiments, the hemostasis valve 6530 can include an auxiliary seal 6574. The auxiliary seal 6574 may be a wiper seal. The seal 6574 may prevent leakage lower pressure.

The auxiliary seal 6574 may include a plurality of openings. The auxiliary seal 6574 can include a central opening 6576A. The auxiliary seal 6574 can include a plurality of openings 6576B (e.g., outer openings or radial openings). The hemostasis valve 6530 can include a cap 6554. The openings 6576B can be configured to receive one or more pins or protrusions 6579 of the cap 6554. The protrusions 6579 can couple with the openings 6576B to maintain the position of (e.g., prevent displacement of) the auxiliary seal 6574. As shown in FIG. 33J shows an embodiment of the auxiliary seal having 4 openings 6576B. FIG. 33K shows an embodiment of the auxiliary seal having 8 openings 6576B. However, other numbers of openings may be used to maintain the position of the seal.

FIG. 33L illustrates a portion of a gear train for rotating an interventional device, such as the catheter 6650. An output gear 6654 can be coupled to a gear 6652 coupled to the interventional device (e.g., catheter 6650). In some embodiments, the interventional device can be a guidewire or an access catheter. In some embodiments, the output gear 6654 can be a worm gear. A worm gear may prevent back driving of the interventional device, for example, in response to torsional strain produced while navigating tortuous anatomy.

### Air Bubble Management

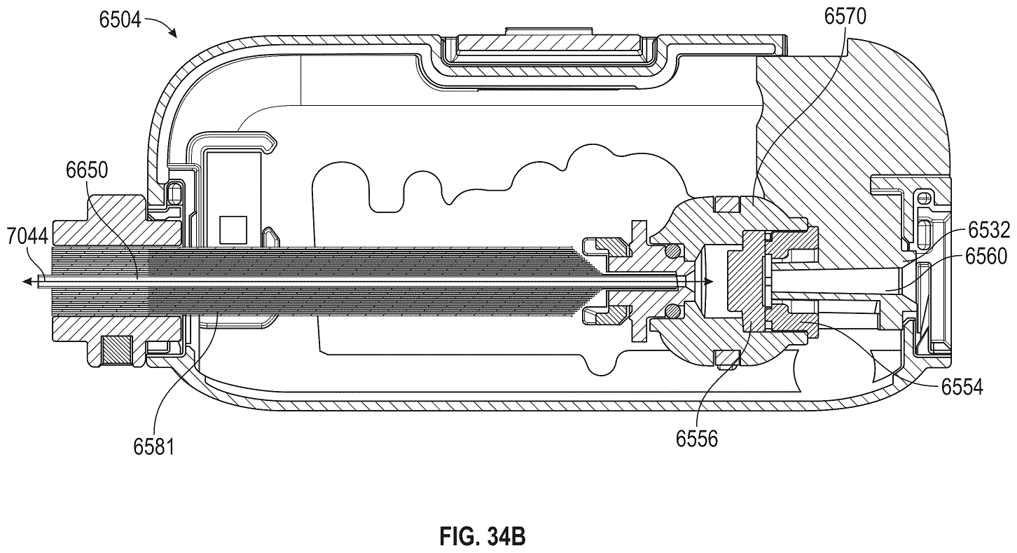

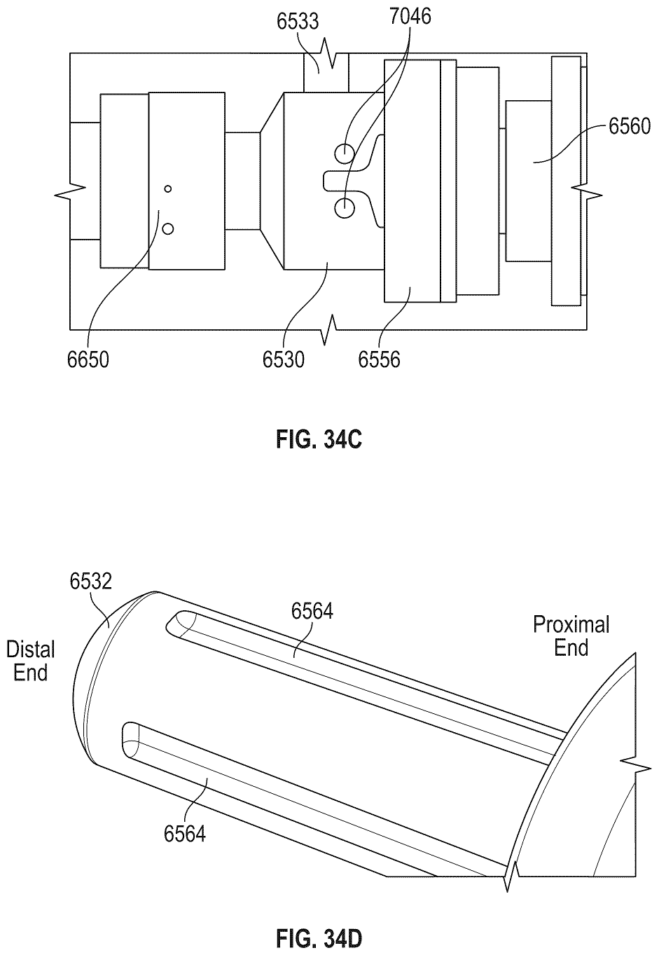

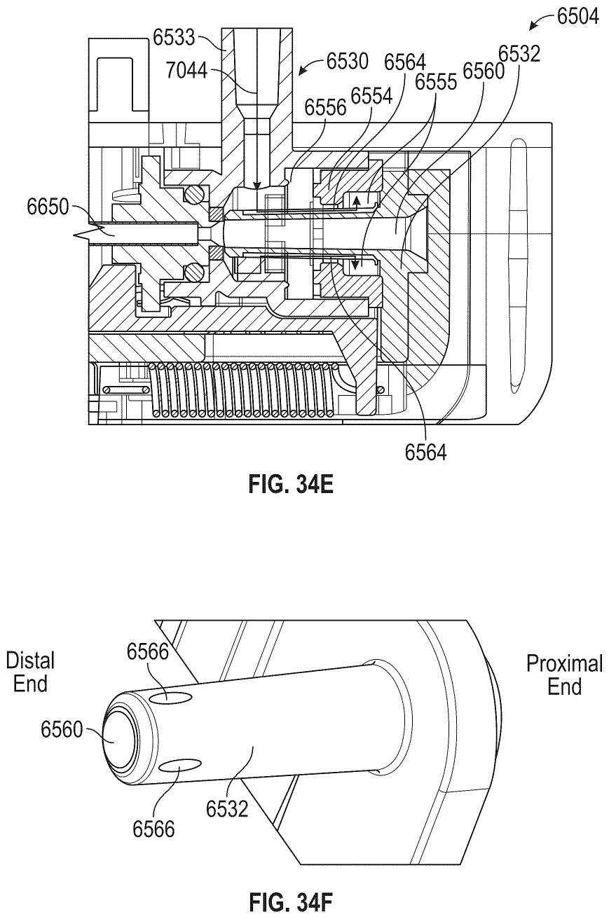

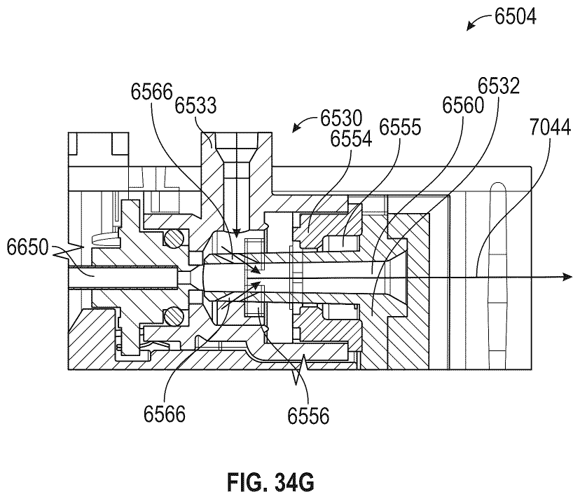

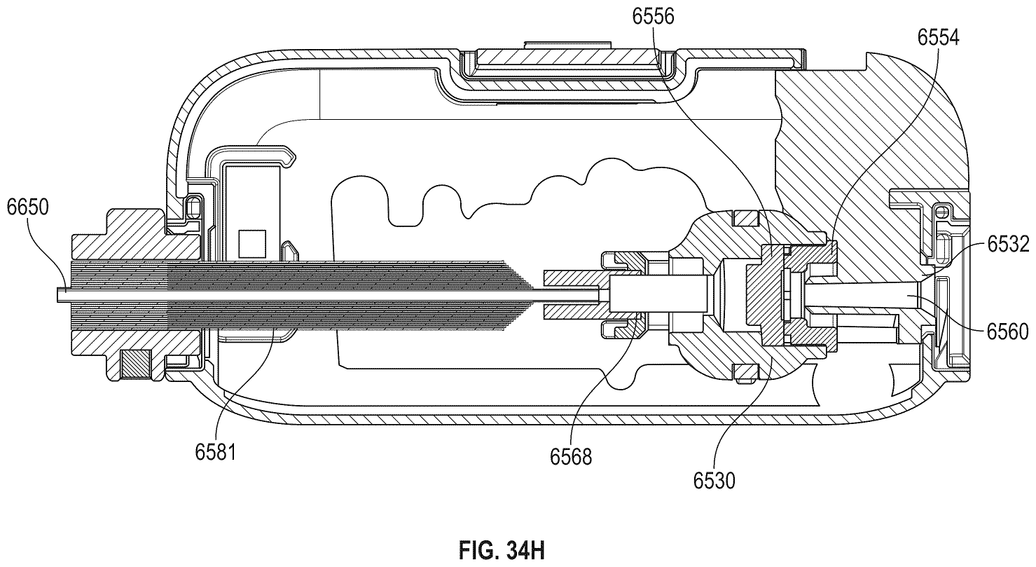

FIGS. 34A-34I illustrate embodiments of mechanisms for removing air bubbles from the fluidics management system (e.g., the fluidics components of a mount and/or hub). While reference may be made to particular embodiments of hub assemblies, the mechanisms for removing air bubbles from a fluidics management system described herein may be part of or used in any hub or hub assembly described herein, including the hub 6004 and/or the hub 6504.

As shown in FIG. 34A, a fluid flow 7044 can flow through the fluidics connector between the mount 6502 and the hub 6504. In some cases, a fluid flow 7044 can be provided from the mount 6502 to the hub 6504 to provide a forward flush. In some cases, fluid (e.g., blood) can be aspirated from the hub 6504 to the mount 6502 as part of a backbleeding process.

As shown in FIGS. 34A-34B, a forward flush can result in outputting a fluid flow 7044 from the mount 6502 into a corresponding interventional device, such as the catheter 6650. In some cases, the interventional device may be inserted into the vascular system of a patient. In some cases, the interventional device may be exposed to an external environment. For example, before inserting the interventional device into the vascular system of the patient. A forward flush be used to release and/or expel air bubbles from the fluidics management system 7000 before inserting the interventional device into the vascular system of the patient. Accordingly, a forward flush can be configured to prime the intravascular device prior to insertion into the vascular system of the patient.

As shown in FIG. 34B, backbleeding can result in aspirating a fluid flow 7044 from the interventional device, hub 6504, and/or mount 6502. In some cases, backbleeding may be performed when the interventional device is inserted into the vascular system of a patient. For example, backbleeding may be performed to aspirate contrast, saline, blood and/or other biological fluids from the interventional device. In some cases, backbleeding may provide a low flow rate of fluids relative to the forward flush. For example, a forward flush may be configured to provide fluid to the interventional device at a higher flow rate than a backbleed is configured to remove fluid from the interventional device. The low flow rate may be inadequate to remove air stuck in the fluidics system, which may result in air bubbles trapped in the fluidics system. For example, FIG. 34C illustrates an example of air bubbles 7046 trapped within the hemostasis valve 6530 of the fluidics management system 7000.

Accordingly, in some cases, providing fluids to the catheter after backbleeding may risk introducing the air bubbles 7046 to the vascular system of the patient. Removing the intravascular device from the vascular system to re-prime the vascular device may be impractical during an operation. Accordingly, a mechanical solution is desired to remove and/or mitigate the air bubbles 7046 from entering the vascular system of the patient. In some cases, applying one or more impulses of force may be effective to release the air bubbles 7046. For example, applying one or more forces to the hemostasis valve 6530 may provide sufficient energy to release the air bubbles 7046. Similarly, applying one or more forces to the seal 6556 via the plunger 6532 may not provide sufficient energy to release the air bubbles 7046. In some cases, changing the geometry of the hemostasis valve 6530 and/or the seal 6556 may be ineffective in encouraging the air bubbles 7046 to release. For example, air bubbles 7046 may remain within the hemostasis valve 6530 regardless of changes to face of the seal 6556 and/or the angle of the fluid port 6533 relative to the hemostasis valve 6530.

In some embodiments, a proximal opening to the catheter can be blocked (e.g., via a valve or other mechanism) and forward flushing can be performed to flush fluid out of a proximal end of the hemostasis valve (e.g., to remove bubbles in the hemostasis valve or mount).

FIGS. 34D-34E illustrate an embodiment of a hub 6504 including a plunger 6532 having one or more longitudinal slots 6564 or other openings extending axially along the outer sidewall of the plunger 6532. The one or more longitudinal slots 6564 can be configured to guide the fluid flow 7044 away from the catheter 6650 and out of the proximal end of the hemostasis valve when the opening of the catheter is blocked.

A plunger including one or more longitudinal slots configured to guide the fluid flow away from the catheter and out of the proximal end of the hemostasis valve when the opening of the catheter is blocked, such as plunger 6532, may be part of or used in any hub or hub assembly described herein, including the hub 6004 and/or the hub 6504.

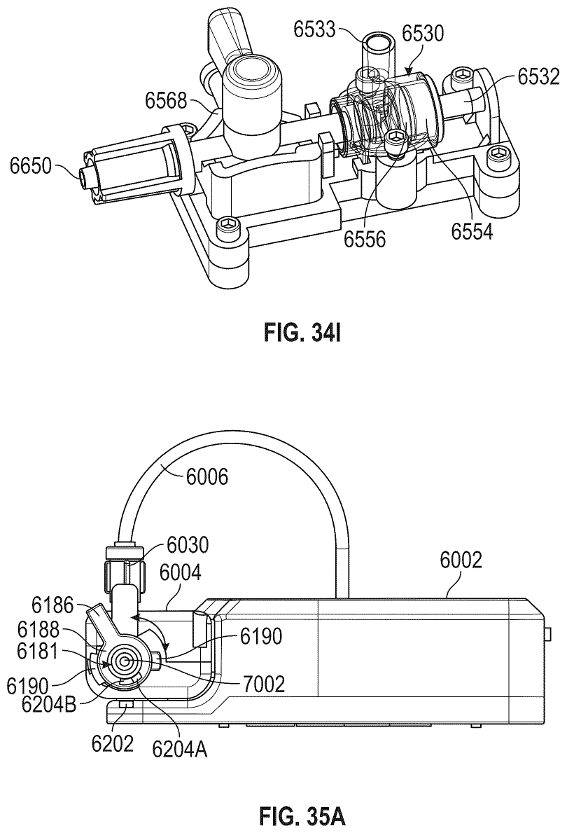

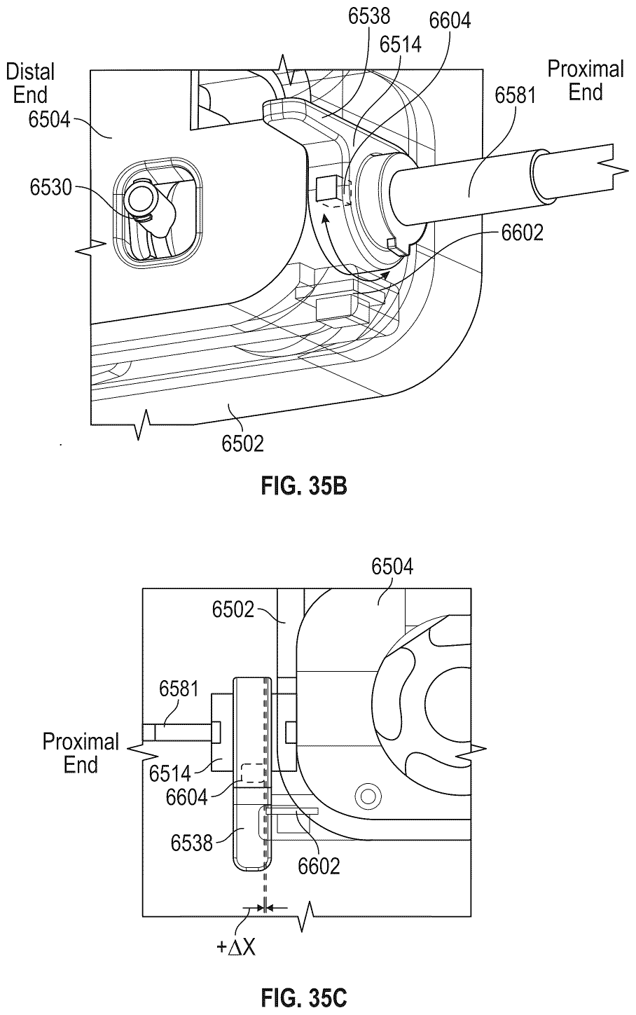

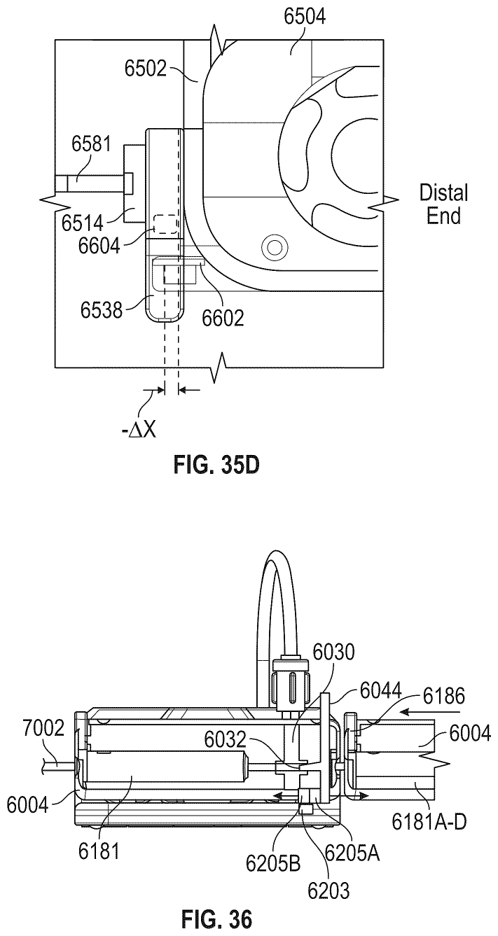

As shown in FIG. 34D, the plunger 6532 can include one or more channel longitudinal channels or slots 6564. The one or more longitudinal slots 6564 can form grooves within the outer sidewall of the plunger 6532. In some cases, the one or more longitudinal slots 6564 may be uniformly and annularly positioned around the outer sidewall of the plunger 6532. As further shown in FIGS. 35D, in some embodiments, the one or more longitudinal slots 6564 may not extend to the proximal end of the plunger 6532. Additionally, in some embodiments, the one or more longitudinal slots 6564 may not access the channel 6560 of the plunger 6532.

As shown in FIG. 34E, when the plunger 6532 is fully depressed within the hub 6504, the proximal end of the plunger 6532 may seal off the catheter 6650 from the hemostasis valve 6530. For example, the volume radially surrounding the plunger 6532 may be scaled off from the catheter 6650. Accordingly, fluid within the hemostasis valve 6530 cannot progress into the vascular system of the patient through the catheter 6650. As discussed in greater detail herein with respect to FIG. 36, a sensor can be included to verify that the plunger 6532 is fully depressed within the hub 6504 and ensure that catheter 6650 is sealed off from the volume radially surrounding the plunger 6532.

In such embodiments, the one or more longitudinal slots 6564 can provide a flow path past the seal 6556 from the hemostasis valve 6530 to a volume 6555. The volume 6555 can extend annularly around the plunger 6532. In some cases, the volume 6555 may be defined at least partially by the cap 6554. When the plunger 6532 is removed from the hemostasis valve 6530, the volume 6555 may be exposed to an external environment. In such embodiments, moving the plunger 6532 proximally may empty the fluid contained within the volume 6555. Accordingly, a forward flush can release air bubbles 7046 without risking the air bubbles 7046 entering the vascular system of the patient.

FIGS. 34F-34G illustrate an embodiment of a hub 6004 including a plunger 6532 having one or more channels 6566 extending proximally through the outer sidewall of the plunger 6532. The one or more channels 6566 can be configured to guide the fluid flow 7044 away from the catheter. A plunger, such as plunger 6532, including one or more channels configured to guide the fluid flow away from the catheter may be part of or used in any hub or hub assembly described herein, including the hub 6004 and/or the hub 6504.

As shown in FIG. 34F, the plunger 6532 can include one or more channels 6566. The one or more channels 6566 can form a conduit through the outer sidewall of the plunger 6532. Accordingly, fluid can access the channel 6560 of the plunger 6532. Accordingly, external fluid can enter the channel 6560 of the plunger 6532 via the one or more channels 6566. In some cases, the one or more channels 6566 can be angled toward the proximal end of the plunger 6532. Angling the one or more channels 6566 towards the proximal end of the plunger 6532 may direct the fluid flow 7044 toward the proximal end of the plunger 6532 and away from the distal end of the plunger 6532. In some cases, the one or more channels 6566 may induce a Venturi-effect to expel fluid from the proximal end of the plunger 6532.

As shown in FIG. 34G, when the plunger 6532 is fully depressed within the hub 6504, the distal end of the plunger 6532 may seal off the catheter 6650 from the hemostasis valve 6530. For example, the volume radially surrounding the plunger 6532 may be sealed off from the catheter 6650. Accordingly, fluid within the hemostasis valve 6530 cannot progress into the vascular system of the patient through the catheter 6650. As discussed in greater detail herein with respect to FIG. 36, a sensor can be included to verify that the plunger 6532 is fully depressed within the hub 6504 and ensure that the catheter 6650 is sealed off from the volume radially surrounding the plunger 6532.

In such embodiments, the one or more channels 6566 can provide a flow path into the channel 6560 of the plunger 6532. The proximal end of the plunger 6532 may be open to the environment to allow the fluid to be expelled. In some cases, fluid flow through the one or more channels 6566 may induce a Venturi-effect. The Venturi-effect may direct the fluid in the proximal direction away from the catheter 6650. Accordingly, a forward flush can release air bubbles 7046 without risking the air bubbles 7046 entering the vascular system of the patient.

FIGS. 34H-34I illustrate embodiments of a hub including a valve positioned between the hemostasis valve and the catheter. The valve is configured to selectively seal the catheter from the hemostasis valve. The embodiments of valves configured to selectively seal the catheter from the hemostasis valve described herein may be part of or used in any hub or hub assembly described herein, including the hub 6004 and/or the hub 6504.

FIG. 34H illustrates top view of an embodiment of a hub 6504 including a valve 6568 positioned between the hemostasis valve 6530 and the catheter 6650. The valve 6568 can be configured to selectively seal the catheter 6650 from the hemostasis valve 6530. In some cases, the valve 6568 can be a pinch valve configured to depress the catheter 6650. In some cases, the valve 6568 can be configured to rotate an inner mechanism to selectively seal the catheter 6650 from the hemostasis valve 6530. For example, the valve 6568 may be a plug valve, a stopcock, or a ball valve. In some cases, the valve 6568 can be a gate valve configured to transversely seal the catheter 6650 from the hemostasis valve 6530. After the valve 6568 seals the interventional device from the hemostasis valve 6530, a forward flush can be performed to drive fluid through the fluidics system within the mount 6502, through the hemostasis valve 6530, and out of a proximal end of the hemostasis valve 6530 to remove air bubbles.

In some embodiments (e.g., for hubs 6504 in which catheter rotation is not desired) the valve 6568 can be actuated between open and closed states in response to an input received by one of the output members 6534 of the mount 6502. Thus, in some embodiments, actuation of the valve 6568 can be controlled by one of the passive torque elements 6507. In some embodiments, one of the output members 6534 can provide an input to the hub 6504 for actuation of the hemostasis valve 6530 and the other output member 6534 can provide an input to the hub 6504 for action of the valve 6568. In other embodiments, an additional output member 6534 or other actuator may be used to actuate the valve 6568.

FIG. 34I illustrates an embodiment of a hub 6504 having a valve 6568. As shown in FIG. 34I, the valve 6568 can be a stopcock. The stopcock can be configured to selectively seal off the catheter 6650. In some embodiments (e.g., for hubs 6504 in which catheter rotation is not desired) the stopcock can be actuated between open and closed states in response to an input received by one of the output members 6534 of the mount 6502. Thus, in some embodiments, actuation of the valve stopcock can be controlled by one of the passive torque elements 6507. In some embodiments, one of the output members 6534 can provide an input to the hub 6504 for actuation of the hemostasis valve 6530 and the other output member 6534 can provide an input to the hub 6504 for action of the stopcock. In other embodiments, an additional output member 6534 or other actuator may be used to actuate the stopcock.

In some cases, the valve 6568 may be robotically actuated based on forward flush and/or backbleeding. For example, the valve 6568 may be automatically actuated to seal off the catheter 6650 after backbleeding. A forward flush may then be performed to release any possible air bubbles. The valve 6568 may then be automatically actuated to unseal the catheter 6650 after a forward flush.

In some embodiments, an additional fluid input line (e.g., a second channel 6732) may be provided to the hub 6504 and positioned to provide additional flushing of the hemostasis valve 6530 (e.g., via an additional connector such as connecter 6006). In some embodiments, the fluid from the additional fluid input line can be directed (e.g., via a nozzle or nozzle-like portion of a fluid input line) to dislodge bubbles within the hemostasis valve 6530. The fluid may be provided to the hemostasis valve 6530 at high pressure (e.g., via a nozzle or nozzle like portion of the fluid input line). An additional stopcock 6528A-B may be used to provide fluid to the additional fluid input line.

### Sensors

The robotic drive system described herein may include one or more sensors configured to verify positions and/or statuses of one or more components within the robotic drive system. In some cases, the robotic drive system may include sensors configured to monitor engagement of the anti-buckling devices, position of the plunger and/or actuation of the hemostasis valve, engagement of a locking mechanism, alignment between mounts and hubs, and/or pressures within the fluidics management system. While reference may be made to particular embodiments of hubs, mounts, and/or hub adapters the embodiments of sensors described herein may be included in any hub, mount, or hub adapter described herein.

FIGS. 35A-35D illustrate sensor configurations for monitoring the engagement of an anti-buckling device extending from a first hub to a more distal hub or a distal attachment along a drive table. The embodiments of sensor configurations for monitoring the engagement of an anti-buckling device extending from a first hub to a more distal hub or a distal attachment along a drive table described herein may be part of or used in any hub adapters described herein.

FIG. 35A depicts an embodiment of an anti-buckling device 6181 of a hub 6004. As described herein, the anti-buckling devices 6181 can be in the form of telescoping tubes. The anti-buckling devices 6181 can extend distally away from its corresponding hub 6004 to a proximal end of another distally located hub and/or a static engagement mechanism. In such cases, the anti-buckling devices 6181 may provide support to one or more interventional devices extending between its hub 6004 and the other distally located hub or the static engagement mechanism.

The anti-buckling device 6181 can include a distal retainer 6188 configured to engage with a proximal retainer of a more distal hub 6004 of a static engagement mechanism. The anti-buckling device 6181 can also include an engagement tab 6186 and one or more locking tabs 6190, which may form part of the distal retainer. The engagement tab 6186 may be manipulated by a user to deploy the anti-buckling devices 6181 and/or to engage with the proximal end of the distally located hub 6004 and/or a static element. The distal retainer 6188 can be configured to rotate about a rotational axis. In some cases, the distal retainer 6188 may rotate about the rotational axis to transition between a locked position and an unlocked position. For example, the distal retainer 6188 may be in a locked state when the engagement tab 6186 is angled away from a vertical axis, as shown in FIG. 34A. In some cases, the distal retainer 6188 may be in an unlocked state when the engagement tab 6186 is aligned with a vertical axis. The one or more locking tabs 6190 can be configured to engage with a corresponding structure of the distal hub. In the unlocked state, the one or more locking tabs 6190 may not contact the corresponding structure of the distal hub. In the locked state, the one or more locking tabs 6190 may contact the corresponding structure of the distal hub to secure the distal retainer 6188 to a proximal retainer of the distal hub.

A sensor 6202 can be located within a mount 6002. The sensor 6202 can be configured to detect the presence of detectable objects 6204A-B positioned on the distal retainer 6188. In some cases, the sensor 6202 may be positioned at a proximal end of a mount 6002 for detecting engagement by an anti-buckling device 6181 extending from a proximally located hub 6004. Accordingly, the sensor 6202 can identify the presence of an anti-buckling device 6181 of a proximally located hub 6004.

In some embodiments, the sensor 6202 can detect that the anti-buckling device 6181 from a proximally located hub 6004 is secured to the hub 6004 coupled to the mount 6002 the sensor is positioned on. For example, the sensor 6202 can detect that the distal retainer 6188 is in the locked position.

As shown in FIG. 35A, the sensor 6202 can be disposed along a surface of the mount 6002 facing its hub 6004. The one or more detectable objects 6204A-B can be positioned within the distal retainer 6188 of another hub (not shown). For example, the distal retainer 6188 can include a first detectable object 6204A and a second detectable object 6204B. In some cases, the one or more detectable objects 6204A-B can be magnets. For example, the first detectable object 6204A can be a first magnet having a first polarity and the second detectable object 6204B can be a second magnet having a second polarity opposite the first magnetic pole. The sensor 6202 can be a hall-effect sensor configured to measure magnetic fields. As shown in FIG. 34A, the first detectable object 6204A and the second detectable object 6204B can be annularly arranged. Accordingly, the sensor 6202 can be configured to detect a change in magnetic polarity as the distal retainer 6188 rotates. In some cases, a threshold value may be indicative of when the distal retainer 6188 is either in a fully locked position or in a fully unlocked position. Accordingly, the sensor 6202 can be configured to determine whether a distal retainer of a proximal hub is secured to the proximal retainer of a more distal hub 6004.

In some embodiments, the detectable objects 6204A and 6204B may have different polarities or other different distinguishable characteristics. The detectable objects 6204A can be positioned about the distal retainer 6188 so that the detectable object 6204A will be aligned with and/or detected by the sensor 6202 when the distal retainer 6188 is in the locked position (e.g., when the anti-buckling device 6181 is secured to the proximal attachment of a hub 6004). The detectable object 6204B can be positioned about the distal retainer 6188 so that the detectable object 6204B will be aligned with and/or detected by the sensor 6202 when the distal retainer 6188 is in the unlocked locked position (e.g., when the anti-buckling device 6181 is not secured to the proximal attachment of a hub 6004).

In some cases, the one or more detectable objects 6204A-B can be radially arranged. For example, a first detectable object 6204A can be positioned radially inward relative to a second detectable object 6204B, and vice versa. In such embodiments, the sensor 6202 can be configured to detect a presence of any magnetic field. A detected output of the one or more detectable objects 6204A-B can be at a maximum value when the one or more detectable objects 6204A-B are rotatably aligned with the sensor 6202. The detected strength of the output of the one or more detectable objects 6204A-B can be at a minimum value as the one or more detectable objects 6204A-B rotate away from the sensor 6202. Accordingly, the position of the one or more detectable objects 6204A-B can be determined based on the relative rotational position of the one or more detectable objects 6204A-B to the sensor 6202. Thus, the radial arrangement can simplify detection of the engagement tab 6186 because the sensor 6202 need not determine the difference between different (e.g., south and north) polarities, but rather the strength of any polarity. The sensor 6202 may be able to detect the presence of the distal retainer 6188 based on a detected output from the one or more detectable objects 6204A-B. The sensor 6202 may also be able detect a locked and unlocked state based on a strength of the magnetic field detected.

FIG. 35B depicts an embodiment of an anti-buckling device 6181 of a hub 6504. FIG. 35B shows an engagement tab 6538 in an unlocked state. As shown, the engagement tab 6538 can be arranged orthogonally from a sensor 6602. The sensor 6602 can be configured to detect the one or more detectable objects 6604 positioned on the distal retainer 6514. The sensor 6602 can be configured to detect the present of the anti-buckling device 6181 (e.g., detect the presence of the distal retainer 6514). The sensor can also be configured to detect the one or more detectable objects as the engagement tab 6538 and distal retainer 6514 rotates. The sensor 6602 may be configured to detect a locked state and an unlocked state, for example, based on a polarity or strength of a magnetic field from the one or more detectable objects 6604. For example, a maximum polarity can be detected when the distal retainer 6514 is in the locked state.

In some cases, the sensor 6602 can be configured to detect the one or more detectable objects 6604 when the sensor 6602 and the one or more detectable objects 6604 are axially aligned. As shown in FIG. 35C, the distal retainer 6514 and the one or more detectable objects 6604 do not axially overlap with the mount 6502 as shown by the distance +ΔX. In some cases, the distal retainer 6514 may not axially overlap with the mount 6502 if the anti-buckling device 6581 is not fully extended between a proximally positioned hub 6504 and the corresponding hub 6504. Accordingly, the sensor 6602 may not detect the presence of the one or more detectable objects 6604 within the distal retainer 6514. As shown in FIG. 35D, the distal retainer 6514 and the one or more detectable objects 6604 do axially overlap with the mount 6502 as shown by the distance-AX. In some cases, the distal retainer 6514 may axially overlap with the mount 6502 if the anti-buckling device 6581 is fully extended between a proximally positioned hub 6504 and the corresponding hub 6504. Accordingly, the sensor 6602 may detect the presence of the one or more detectable objects 6604 only when the anti-buckling device 6581 is fully deployed.

FIG. 36 illustrates a sensor configuration for monitoring the position of the plunger and/or actuation of the hemostasis valve of a hub. The embodiments of sensor configurations for monitoring the position of a plunger and/or actuation of a hemostasis valve described herein may be part of or used in any hub adapter described herein. The sensor can be used to detect if the hemostasis valve 6030 is opened or closed. As described herein, the plunger 6032 can be used to mitigate and/or release air bubbles after the catheter 7002 is inserted into the vascular system of a patient. Accordingly, tracking the position of the plunger 6032 may be helpful in preventing air bubbles from entering the vascular system of the patient.

As shown in FIG. 36, the mount 6002 may include a sensor 6203 configured to detect the presence of one or more detectable objects 6205A-B. As further shown in FIG. 36, the plunger 6032 may include a first detectable object 6205A and a second detectable object 6205B. In some cases, the one or more detectable objects 6205A-B can be magnets. For example, the first detectable object 6205A can be a first magnet having a first polarity and the second detectable object 6205B can be a second magnet having a second polarity opposite the first magnetic pole. The sensor 6203 can be a hall-effect sensor configured to measure magnetic fields.

As further shown in FIG. 36, the first detectable object 6205A and the second detectable object 6205B may be axially arranged within or coupled to the tab 6044. As described herein, the plunger 6032 can extend from the tab 6044. The tab 6044 can be actuated by movement of a slider to transition the seal of the hemostasis valve 6030 between various states. For example, an input gear can be coupled to the rotating body configured to rotate so that a pin can move within a slot of the slider to cause linear motion of the slider, resulting in linear motion of the plunger 6032. A spring can be linearly depressed as the slider is linearly actuated in the direction of the spring. The spring can act to bias the slider to a first position. Accordingly, the plunger 6032 and the tab 6044 can be configured to translate axially across the sensor 6203 disposed within the mount 6002. The sensor 6203 can detect the axial position of the one or more detectable objects 6205A-B. The axial position of the plunger 6032 may be fixed relative to the axial position of the one or more detectable objects 6204A-B. Accordingly, the sensor 6202 can be configured to detect the axial position of the plunger 6032.

In some embodiments, the robotic control system may be configured to provide a forward flush only when the sensor 6203 detects that the plunger 6032 is fully depressed such that the plunger 6032 blocks fluid from flowing into the catheter 7002 as discussed herein.

In some embodiments, the pressure sensor 6236 may be used to detect if the hemostasis valve 6030 is open or closed. For example, when the hemostasis valve 6030 is opened, the pressure on the pressure sensor may change from body pressure to atmospheric pressure. When the hemostasis valve is closed the pressure on the pressure sensor may change from atmospheric pressure to body pressure.

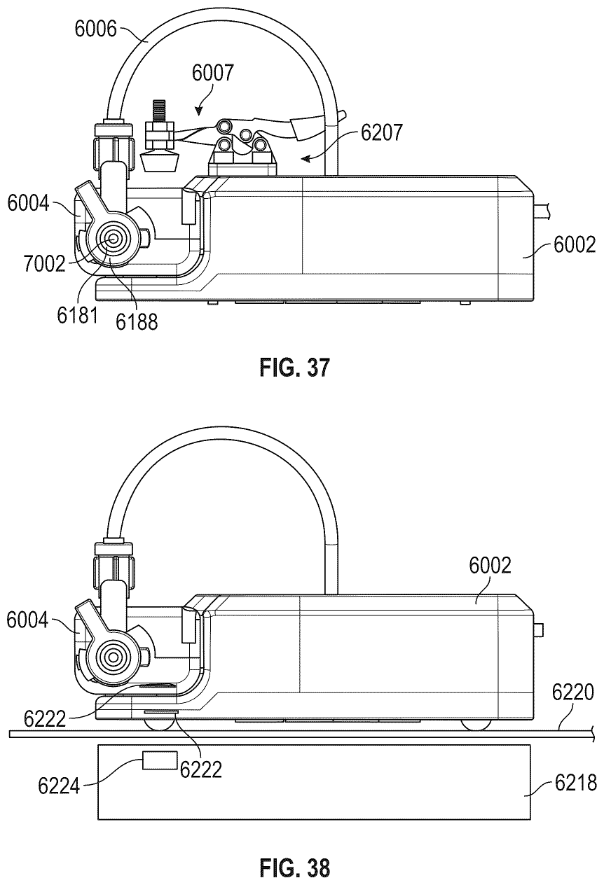

FIG. 37 illustrates a sensor configuration for monitoring the position of a hub relative to a corresponding mount. For example, the sensor configuration may monitor whether the hub is fully secured within the corresponding mount. The embodiments of sensor configurations for monitoring the position of a hub relative to a corresponding mount described herein may be part of or used in any hub adapter described herein.

FIG. 37 illustrates an embodiment of a mount 6002 including a clamp mechanism 6007 as described herein. For example, the clamp mechanism 6007 may include a lever arm. In some embodiments, the lever arm may be configured to rotate about a pivot point. The lever arm can be biased toward the hub 6004. In the biased state, the clamp mechanism 6007 may secure a corresponding hub 6004 in a mounted position. The clamp mechanism 6007 may transition to an unbiased position by depressing the lever arm on a side of a pivot point opposite the hub 6004. The clamp mechanism 6007 can further include a binary position sensor 6207. The binary position sensor 6207 can determine whether the clamp mechanism 6007 is either in the biased state or the unbiased state.

FIG. 38 illustrates a sensor configuration for monitoring alignment between a mount and a corresponding hub adapter. A mount can be magnetically coupled to the hub adapter or carriage which acts as a shuttle by advancing proximally or distally along a track in response to operator instructions or controller manipulations. The hub adapter includes at least one drive magnet configured to couple with a driven magnet carried by the mount. This provides a magnetic coupling between the drive magnet and driven magnet through a sterile barrier 6220 such that the mount is moved across the top of the sterile barrier 6220 in response to movement of the hub adapter outside of the sterile field. It can be vital that the mount is magnetically coupled to the correct hub adapter and/or that the mount is properly coupled with the correct hub adapter. The embodiments of sensor configuration for monitoring alignment between a mount and a corresponding hub adapter described herein may be part of or used in any hub assembly and hub adapter described herein.

FIG. 38 illustrates an embodiment where the mount 6002 can include one or more wireless communication devices 6222. In some cases, the one or more wireless communication devices 6222 can include radio frequency identification (“RFID”) and/or near field communication (“NFC”) tags. The hub adapter 6218 can include a corresponding wireless communication device 6224 configured to identify a signal from the one or more wireless communication devices 6222 positioned on the mount 6002. In some cases, the wireless communication devices 6222, 6224 can be configured to identify the corresponding mount 6002 and/or hub adapter 6218. In some cases, one or more mounts 6002 can each include one or more sensors configured to detect one or more detectable objects positioned on one or more hub adapters 6218, and vice versa. For example, one or more mounts 6002 can each include one or more detectable objects configured to be detected by one or more sensors positioned on one or more hub adapters 6218 as described in U.S. patent application Ser. No. 18/678,766, filed May 30, 2024, titled MAGNETIC COUPLING THROUGH A STERILE FIELD BARRIER, the entire content of which is incorporated by reference herein for all purposes and forms as part of this specification.

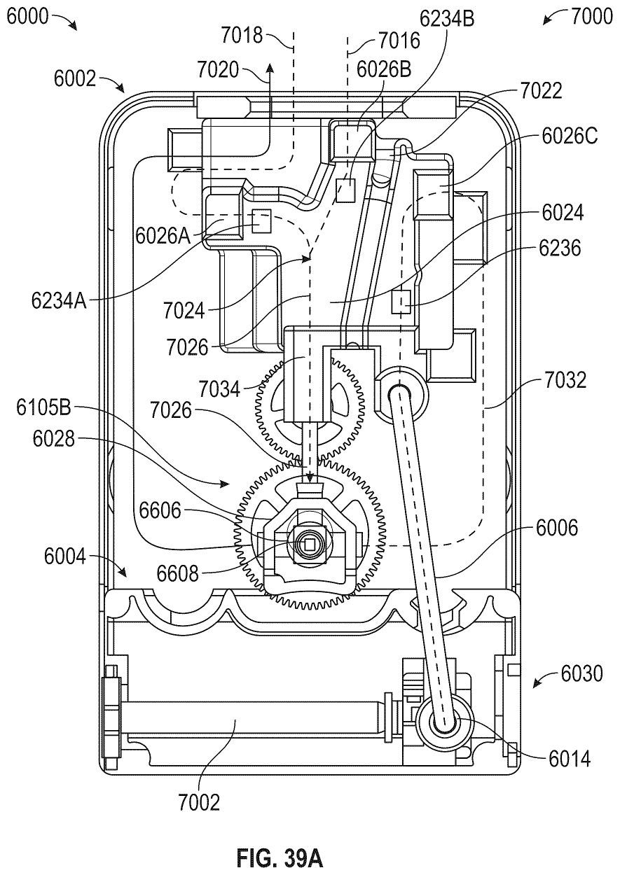

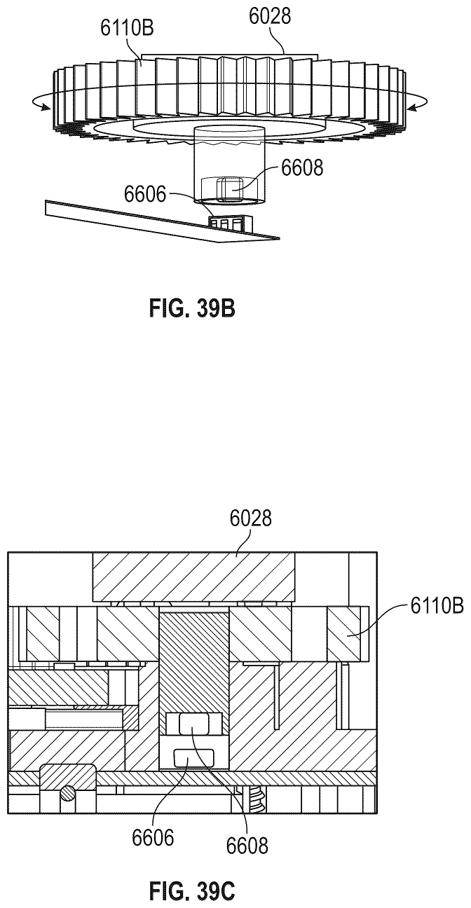

FIGS. 39A-39C illustrate mounts having sensors measuring components of fluidics management systems. The sensors can be configured to monitor air bubbles in the contrast tube, air bubbles in the saline tube, a rotational position of one or more 3-way valves (“stopcocks”), and/or pressure within the fluidics management system. The embodiments of sensor configurations for measuring components of fluidics management systems described herein may be part of or used in any mount or hub assembly described herein.

As shown in FIG. 39A, a mount 6002 can include one or more air bubble sensors. A first air bubble sensor 6234A can be in fluid communication with the saline tube 7018. The first air bubble sensor 6234A can be configured to monitor the saline tube 7018 for air bubbles. A second air bubble sensor 6234B can be in fluid communication with the contrast tube 7016. The second air bubble sensor 6234B can be configured to monitor the contrast tube 7016 for air bubbles. In some embodiments, the fluidics management system 7000 may actuate a valve in response to a positive identification of an air bubble detected by the first air bubble sensor 6234A or the second air bubble sensor 6234B.

The mount 6002 can further include a pressure sensor 6236. The pressure sensor 6236 can be configured to measure pressure within one or more of the fluidics tubes. In some cases, the pressure sensor 6236 can be in fluid communication with an output of the fluidics management system 7000. For example, the pressure sensor 6236 can be configured to monitor the pressure within the fluidics connector 6006.

In some embodiments, a mount 6002 can include a sensor 6606 and one or more detectable objects 6608. FIGS. 39B-39C illustrate a perspective view and a side view of a 3-way valve 6528A having a detectable object 6608 monitored by a sensor 6606. The one or more detectable objects 6608 can be positioned along a shaft of one of the one or more 3-way valves 6528A-B. The sensor 6606 can be configured to measure the rotational position of the detectable object 6608 as the 3-way valve 6028A-B rotates. Accordingly, the sensor 6606 can be configured to detect the rotational position of the 3-way valve 6028A-B. Detecting the rotational position may provide for redundancy and safety mitigation to confirm the position of the 3-way valves 6028A-B before changing a fluid state (e.g., injecting, aspirating, etc.).

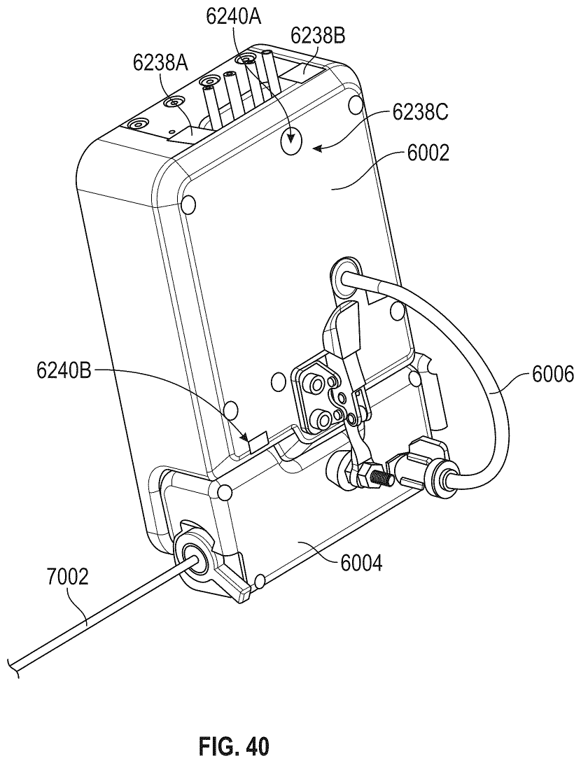

FIG. 40 illustrates a mount 6002 having buttons and status indicators. In some embodiments, the mount 6002 can include one or more manual actuation buttons 6238A-C. The one or more manual actuation buttons 6238A-C can be configured to axially drive the mount 6002 along a drive table and/or operate a fluidics management system. In some cases, a first manual actuation button 6238A can be configured to drive the mount 6002 distally axially along the drive table, a second manual actuation button 6238B can be configured to drive the mount 6002 proximally axially along the drive table, and a third manual actuation button 6238C can be configured to flush the fluidics management system. In some cases, the one or more manual actuation buttons 6238A-C can be depressed by an operator located within the operating room. For example, depressing one or more manual actuation buttons 6238A-C can transmit an electrical signal to a motor. The electrical signal can be configured to actuate a hub adapter below a sterile barrier and/or to actuate one or more valves within the mount for controlling fluid flow through the fluidics management system. In some cases, the first manual actuation button 6238A can be positioned near a first side of the mount 6002, the second manual actuation button 6238B can be positioned near a second side of the mount 6002 opposite the first manual actuation button 6238A. For example, the first manual actuation button 6238A can be positioned on a distal side of the mount 6002 and the second manual actuation button 6238B can be positioned on a proximal side of the mount 6002. The third manual actuation button 6238C can be positioned along a top surface of the mount 6002.

The mount 6002 can further include one or more status indicators 6240A-B. The one or more status indicators 6240A-B can provide an indication of one or more statuses within the robotic control system. In some embodiments, the one or more status indicators 6240A-B can include a light emitting diode (LED) configured to output a light corresponding to one or more possible status outputs. The one or more status indicators 6240A-B can include a first status indicator 6240A and a second status indicator 6240B. In some cases, the first status indicator 6240A can indicate whether fluids are being supplied to a hub. For example, a first status indicator 6240A can be configured to indicate whether saline, contrast, and/or aspiration is being provided to an intravascular device. In some cases, the second status indicator 6240B can be configured to indicate whether the hub 6004 is coupled to the mount 6002.

Additional details regarding sensors, hub assemblies, and alignment of hub assemblies are disclosed in U.S. patent application Ser. No. 18/389,628, entitled Hub Sensing Through A Sterile Barrier In A Robotic Catheter Assembly, filed Dec. 19, 2023, U.S. patent application Ser. No. 18/545,687, entitled System With Removable Hubs For Manual And Robotic Procedure, filed Dec. 19, 2023, and U.S. patent application Ser. No. 18/678,766, entitled Magnetic Coupling Through a Sterile Field Barrier, filed May 30, 2024, and U.S. patent application Ser. No. 18/784,630, entitled System For Remote Medical Procedure, filed Jul. 25, 2024, each of which is hereby expressly incorporated by reference in its entirety herein.

The foregoing represents example embodiments of a robotic control system. A wide variety of different robotic control system constructions can be made, for supporting and axially advancing and retracting two or three or four or more assemblies to robotically drive interventional devices, as will be appreciated by those of skill in the art in view of the disclosure herein.

Various systems and methods are described herein primarily in the context of a neurovascular access or procedure. However, the inventors contemplate applicability of the disclosed catheters, systems, and methods to any of a wide variety of alternative applications, including within the coronary vascular or peripheral vascular systems as well as other hollow organs or tubular structures in the body.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein.

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A hub assembly, comprising:
a mount being axially movable along a drive surface;
a catheter hub having a catheter, the catheter hub removably couplable with the mount; and
a fluidics connector in fluid communication with the mount and the catheter hub to provide fluid and vacuum through the connector to the catheter hub.

2. The hub assembly of claim 1, wherein the mount is configured to provide saline, contrast, and vacuum through the connector to the catheter hub.

3. The hub assembly of claim 2, wherein the mount comprises a saline channel, a contrast channel, and a vacuum channel for providing saline, contrast, and vacuum to the catheter hub.

4. The hub assembly of claim 3, wherein the mount comprises a robotically actuated three-way valve coupled to a vacuum channel and a saline-contrast channel, wherein the three-way valve is configured to selectively place the vacuum channel and the saline-contrast channel in fluid communication with the catheter hub.

5. The hub assembly of claim 4, wherein the mount comprises a passive torque subsystem comprising a passive torque element configured to drive the three-way valve.

6. The hub assembly of claim 5, wherein the passive torque element is configured to be actuated by an active torque element of an active torque subsystem separated from the passive torque element by a sterile barrier.

7. The hub assembly of claim 6, wherein the passive torque element comprises at least one magnet configured to rotate in response to rotation of at least one magnet of the active torque element.

8. The hub assembly of claim 4, further comprising a second robotically actuated three-way valve coupled to the saline channel and the contrast channel and configured to selectively place the saline channel and the contrast channel in fluid communication with the saline-contrast channel.

9. The hub assembly of claim 1, wherein the mount is configured to transfer motion to the catheter hub to rotate the catheter when the catheter hub is coupled to the mount.

10. The hub assembly of claim 9, wherein the catheter hub comprises a gear train coupled to the catheter, wherein the mount comprises a gear train configured to couple to the gear train of the catheter hub so that actuation of the gear train of the mount causes rotation of the catheter.

11. The hub assembly of claim 1, wherein the catheter hub comprises a hemostasis valve, wherein the mount is configured to transfer motion to the catheter hub to actuate the hemostasis valve when the catheter hub is coupled to the mount.

12. The hub assembly of claim 11, wherein the catheter hub comprises a gear train coupled to the hemostasis valve, wherein the mount comprises a gear train configured to couple to the gear train of the catheter hub so that actuation of the gear train of the mount causes actuation of the hemostasis valve.

13. The hub assembly of claim 11, wherein the mount comprises a manual actuator configured to be actuated by a user to actuate the hemostasis valve.

14. The hub assembly of claim 13, wherein the catheter hub comprises a manual actuator configured to be actuated by a user when the catheter hub is uncoupled from the mount to actuate the hemostasis valve.

15. The hub assembly of claim 1, wherein the mount comprises one or more electrical connector pins configured to be received within the catheter hub and to establish an electrical circuit to provide electrical and data communication between the mount and the catheter hub.

16. The hub assembly of claim 1, wherein the catheter hub comprises a hemostasis valve configured to receive fluid and vacuum from the mount.

17. The hub assembly of claim 16, wherein the catheter hub comprises a secondary valve positioned between the hemostasis valve and a proximal end of the catheter, wherein the secondary valve is configured to selectively block fluid communication between the hemostasis valve and a lumen of the catheter.

18. The hub assembly of claim 16, wherein the catheter hub further comprises a plunger actuatable to transition a seal of the hemostasis valve between various states, wherein the plunger is axially movable to a distal position wherein the plunger blocks fluid communication between the hemostasis valve and a lumen of the catheter.

19. The hub assembly of claim 18, wherein the plunger comprises one or more channels or slots configured to direct fluid away from the lumen of the catheter when the plunger blocks fluid communication between the hemostasis valve and the lumen of the catheter.

20. The hub assembly of claim 16, wherein the hemostasis valve comprises a seal, a cap comprising a plurality of pins, and an auxiliary seal positioned between the seal and the cap and comprising a plurality of openings configured to receive the plurality of pins of the cap.
